(19) United States
(12) Patent Application Publication
(10) Pub. No.: US 2026/0097076 A1
(43) Pub. Date: Apr. 9, 2026

(54) CHIMERIC ANTIGEN RECEPTORS SPECIFIC TO B-CELL MATURE ANTIGEN (BCMA) AND/OR TRANSMEMBRANE ACTIVATOR AND CAML INTERACTOR (TACI)

(71) Applicant: Elpis Biopharmaceuticals, Lexington, MA (US)

(72) Inventors: Yan CHEN, Lexington, MA (US); Garima AGRAHARI, Lexington, MA (US); Kehao ZHAO, Lexington, MA (US); Jenna NGUYEN, Lexington, MA (US); Keming ZHANG, Lexington, MA (US); Ning JIANG, Lexington, MA (US); Katie O'CALLAGHAN, Lexington, MA (US)

(21) Appl. No.: 19/112,720

(22) PCT Filed: Sep. 20, 2023
(86) PCT No.: PCT/US2023/074658

§ 371 Date: Mar. 18, 2025

Related U.S. Application Data
(60) Provisional application No. 63/376,530, filed on Sep. 21, 2022.

(51) Int. Cl. A61K 35/17; A61K 40/11; A61K 40/15; A61K 40/31; A61K 40/42; A61P 35/00; C07K 14/705; C07K 14/725; C07K 16/28; C12N 15/86

(52) U.S. Cl. CPC A61K 35/17; A61K 40/11; A61K 40/15; A61K 40/31; A61K 40/4215; A61P 35/00; C07K 14/7051; C07K 14/70521; C07K 14/70575; C07K 14/70578; C07K 16/2878; C12N 15/86; A61K 2239/13; A61K 2239/17; A61K 2239/21; A61K 2239/29; C07K 2317/565; C07K 2317/622; C07K 2319/02; C12N 2740/15043

(57) ABSTRACT

Genetically engineered immune cells (e.g., T cells or NK cells) expressing anti-BCMA, anti-TACI, or anti-BCMA/anti-TACI chimeric antigen receptors and uses thereof in cancer therapy. In some embodiments, the genetically engineered immune cells may be armored CAR-T or CAR-NK cells, which further express an armor polypeptide for enhancing CAR-T or CAR-NK cell features.

### CROSS-REFERENCE TO RELATED APPLICATIONS

This application claims the benefit of priority to U.S. Provisional Patent Application No. 63/376,530, filed Sep. 21, 2022, which is hereby incorporated by reference in its entirety.

### SEQUENCE LISTING

The instant application contains a Sequence Listing which has been filed electronically in XML format and is hereby incorporated by reference in its entirety. Said XML copy, created on Sep. 20, 2023, is named 112139-0073-7007WO00_SEQ.XML and is 197,654 bytes in size.

### BACKGROUND OF THE INVENTION

Chimeric antigen receptor (CAR-T) T cells are genetically engineered T cells expressing an artificial T cell receptor for use in immunotherapy. The artificial T cell receptor (known as chimeric antigen receptor) can specifically bind disease cell antigens, such as cancer antigens. Upon binding to the disease cell, the CAR-T cells would be activated and eliminate the disease cell.

While CAR-T cell therapy have demonstrated efficacy in treatment of a few hematological cancers, efficacy of the treatment may be affected by various factors, for example, tumor antigen escape, for example, the expression level of the tumor antigen may reduce to a level that CAR-T cells cannot engage and mediate cytotoxic activity. In some instances, tumor cells may escape killing by expressing an alternative form of the target antigen that lacks the binding epitope to the CAR. In other instances, tumor cells may escape killing by switching to a genetically related but phenotypically different disease (so called lineage switch).

Accordingly, it is of great interest to develop improved CAR-T approaches to address such challenges.

### SUMMARY OF THE INVENTION

The present disclosure is based, at least in part, on the development of anti-BCMA monospecific, anti-TACI monospecific, and anti-BCMA/anti-TACI bi-specific chimeric antigen receptors and CAR-T cells expressing such for successful inhibition of tumor growth in an animal model. Accordingly, provided herein are such CAR constructs, immune cells expression such, and therapeutic uses in cancer therapy.

In some aspects, the present disclosure provides a bi-specific chimeric antigen receptor (CAR) specific to B-cell mature antigen (BCMA) and transmembrane activator and CAML interactor (TACI), the bi-specific CAR comprising: (a) a first antigen binding moiety specific to TACI; (b) a second antigen binding moiety specific to BCMA; (c) a co-stimulatory signaling domain; and (d) a cytoplasmic signaling domain.

In some instances, the first antigen binding moiety specific to TACI of (a) comprises a heavy chain variable region (V_{H}) and a light chain variable region (V_{L}). The V_{H} comprise the same heavy chain CDRs as those in a reference antibody, and the V_{L} comprise the same heavy chain CDRs as those in the reference antibody, which includes TC-01, TC-02, TC-03, or TC-04. In some examples, the reference antibody is TC-01. In some examples, the V_{H} and V_{L} of the first antigen binding moiety specific to TACI are identical to the V_{H} and V_{L} of the reference antibody.

In some examples, the first antigen binding moiety specific to TACI is a single chain variable fragment (anti-TACI scFv). In specific examples, the anti-TACI scFv comprises an amino acid sequence of any one of SEQ ID NOs: 75, 84, 93, and 102 (e.g., SEQ ID NO: 75).

In some embodiments, the second antigen binding moiety specific to BCMA of (a) comprises a heavy chain variable region (V_{H}) and a light chain variable region (V_{L}). The V_{H} may comprise:
(hi) a heavy chain complementarity determining region (CDR) 1, which comprises X₁YX₂MH, in which X₁ is S or D, and X₂ is A or G;
(hii) a heavy chain CDR2, which comprises
   (hii-a) X₃IX₄YDGSX₅KYYADSVKG (SEQ ID NO: 1), in which X₃ is V or F, X₄ is S or R, and X₅ is D or N;
   (hii-b) FIRSKAYGGTTEYAASVKG (SEQ ID NO: 27); or
   (hii-c) GISWNSGSIGYADSVKG (SEQ ID NO: 43); and
(hiii) a heavy chain CDR3, which comprises

(hiii-a)
(SEQ ID NO: 56)
DEHQVVPNYRFDF,
(hiii-b)
(SEQ ID NO: 35)
DWEDPLYYYDTPF,
(hiii-c)
(SEQ ID NO: 16)
DWDYYDSSGYYPDALGI,
(hiii-d)
(SEQ ID NO: 9)
DLWDGIVGAPAGY,
(hiii-e)
(SEQ ID NO: 22)
DLTTITPGY,
(hiii-f)
(SEQ ID NO: 63)
DLWEFGGDYADY,
(hiii-g)
(SEQ ID NO: 28)
GPHYDILTSNWFDP,
or

(hiii-h) VQX₆PGAFDI (SEQ ID NO: 181), in which X₆ is P or S; and

Alternatively or in addition, the V_{L} may comprise:
(li) a light chain CDR1, which comprises

(li-a)
(SEQ ID NO: 58)
SGSGSNIGSNDVS,

(li-b) QASQDIX₇NYLN (SEQ ID NO: 2), in which X₇ is N or S, or
(li-c) RX₈X₉X₁₀ISSYLX₁₁ (SEQ ID NO: 3), in which X₈ is A or S, X₉ is S or T, X₁₀ is G or S, and X11 is G or N;
(lii) a light chain CDR2, which comprises

(lii-a)
(SEQ ID NO: 59)
WNDQRPS,

(lii-b) DASNX₁₂ET (SEQ ID NO: 4), in which X₂ is L or V, or
(lii-c) AX₁₃SX₁₄LQS (SEQ ID NO: 5), in which X₁₃ is A or T, and X₁₄ is S or T; and
(liii) a light chain CDR3, which comprises

(liii-a)
(SEQ ID NO: 60)
AAWDDSLNGWV,

(liii-b) QQYDX₁₅LPX₁₆T (SEQ ID NO: 6), in which X₁₅ is K or N, and X₁₆ is F, L, or Y,

(liii-c)
(SEQ ID NO: 32)
QHSYSTPHT,
or
(liii-d)
(SEQ ID NO: 48)
QQLYS.

In some instances, the V_{H} of the second antigen binding moiety specific to BCMA comprise the same heavy chain CDRs as those in a reference antibody, and/or the V_{L} of the second antigen binding moiety specific to BCMA comprise the same heavy chain CDRs as those in the reference antibody, which include BC-01, BC-02, BC-03, BC-04, BC-05, BC-06, BC-07, BC-08 or BC-09. In some examples, the reference antibody is BC-06. In some examples, the V_{H} and V_{L} of the first antigen binding moiety specific to BCMA are identical to the V_{H} and V_{L} of the reference antibody.

In some examples, the first antigen binding moiety specific to BCMA is a single chain variable fragment (anti-BCMA scFv). In specific examples, the anti-BCMA scFv comprises an amino acid sequence of any one of SEQ ID NOs: 15, 20, 25, 34, 41, 50, 53, 62, and 66 (e.g., SEQ ID NO: 50).

Any of the bi-specific CARs disclosed herein may comprise a co-stimulatory signaling domain. Examples include, but are not limited to, CD28, 4-1BB, OX40, ICOS, CD27, CD40, or CD40L. Alternatively or in addition, the bi-specific CAR may comprise a cytoplasmic signaling domain, which may be from CD3ζ.

In some specific examples, the bi-specific CAR comprises a fusion polypeptide comprising, from N-terminus to C-terminus, (i) the first antigen binding moiety, (ii) the second antigen binding moiety, (iii) the co-stimulatory signaling domain, and (iv) the cytoplasmic signaling domain. Alternatively, the bi-specific CAR may comprise a fusion polypeptide comprising, from N-terminus to C-terminus, (i) the second antigen binding moiety, (ii) the first antigen binding moiety, (iii) the co-stimulatory signaling domain, and (iv) the cytoplasmic signaling domain. Any of the bi-specific CARs disclosure herein may further comprise a hinge domain and a transmembrane domain, which are located between (ii) and (iii). In some instances, the bi-specific CAR may further comprise a peptide linker connecting the first antigen binding moiety and the second antigen binding moiety. Examples include GGGGS (SEQ ID NO: 104), GGGGSGGGGS (SEQ ID NO: 105), GGGGSGGGGSGGGGS (SEQ ID NO: 106), or GSTSGSGKPGSGEGSTKG (SEQ ID NO: 107).

Any of the bi-specific CARs disclosed herein may further comprise a signal peptide at the N-terminus.

In specific examples, the bi-specific CAR disclosed herein may comprise an extracellular bi-specific antigen binding domain, which may comprise the amino acid sequence of SEQ ID NO: 163 or 164. In one example, the bi-specific CAR may comprise the amino acid sequence of SEQ ID NO: 165 or 166.

In some aspects, provided herein is a nucleic acid or a set of nucleic acids, which collectively encode any of the bi-specific CARs disclosed herein. In some instances, the nucleic acid comprises a first nucleotide sequence encoding the bi-specific CAR. In some embodiments, the nucleic acid may further comprise a second nucleotide sequence encoding an armor polypeptide as disclosed herein, which enhances T cell functionality, and a third nucleotide sequence encoding a self-cleaving peptide, which is located between the first and second nucleotide sequences. The nucleic acid or set of nucleic acids may be an expression vector(s), optionally a viral vector(s).

Exemplary armor polypeptide includes, but are not limited to, IL-2, IL-5, IL-15, a co-stimulatory ligand, an anti-PDL1 antibody, or a fusion polypeptide comprising the anti-PDL1 antibody. In some instances, the armor polypeptide is a fusion protein comprising an anti-PDL1 antibody (e.g., a single chain variable fragment (scFv)) and an IL-2 polypeptide. Exemplary armor polypeptides are provided in Table 11, below, each of which is within the scope of the present disclosure.

Further, provided herein is a genetically engineered immune cell, which expresses the bi-specific CAR disclosure herein, and optionally further express the armor polypeptide also disclosed herein. Such genetically engineered immune cells may comprise any of the nucleic acids disclosed herein that encode the bi-specific CAR and optionally the armor polypeptide. In some embodiments, the genetically engineered immune cell can be a T cell, an NK cell, or a macrophage. In one example, the immune cell is a T cell. In specific examples, the genetically engineered immune cell provided herein (e.g., a T cell or an NK cell) may express the bispecific CAR of SEQ ID NO: 165 or 166 and an armor polypeptide, for example, an anti-PDL1-IL2 fusion such as SEQ ID NO: 173 or 174, or an IL2 polypeptide such as SEQ ID NO: 177, 178, 179, or 180.

In other aspects, the present disclosure features an anti-TACI chimeric antigen receptor (CAR), comprising an extracellular antigen binding domain specific TACI, a co-stimulatory signaling domain, and a cytoplasmic signaling domain; wherein the extracellular antigen binding domain specific to BCMA is as disclosed herein. In some embodiments, the co-stimulatory signaling domain is from a co-stimulatory molecule selected from the CD28, 4-1BB, OX40, ICOS, CD27, CD40, or CD40L. Alternatively or in addition, the cytoplasmic signaling domain is from CD3ζ. In some examples, the anti-TACI CAR may further comprise a hinge domain and/or a transmembrane domain between the extracellular antigen binding domain specific to TACI and the co-stimulatory domain. In some examples, the anti-TACI CAR may further comprise both the hinge domain and the transmembrane domain. The anti-TACI CAR may further comprise a spacer between the hinge domain and the transmembrane domain. In specific examples, the anti-TACI CAR disclosed herein may comprise the amino acid sequence of any one of SEQ ID NOs: 143-162 (e.g., SEQ ID NO: 143 or 144).

In other aspects, the present disclosure features an anti-BCMA chimeric antigen receptor (CAR), comprising an extracellular antigen binding domain specific BCMA, a co-stimulatory signaling domain, and a cytoplasmic signaling domain; wherein the extracellular antigen binding domain specific to BCMA is as disclosed herein. In some embodiments, the co-stimulatory signaling domain is from a co-stimulatory molecule selected from the CD28, 4-1BB, OX40, ICOS, CD27, CD40, or CD40L. Alternatively or in addition, the cytoplasmic signaling domain is from CD3ζ. In some examples, the anti-BCMA CAR may further comprise a hinge domain and/or a transmembrane domain between the extracellular antigen binding domain specific to BCMA and the co-stimulatory domain. In some examples, the anti-BCMA CAR may comprise both the hinge domain and the transmembrane domain. The anti-BCMA CAR may further comprise a spacer between the hinge domain and the transmembrane domain. In specific examples, the anti-BCMA CAR disclosed herein may comprise the amino acid sequence of any one of SEQ ID NOs: 119-142 (e.g., SEQ ID NO: 127 or 128(.

Also provided herein is a nucleic acid, comprising a first nucleotide sequence encoding an anti-TACI CAR as set forth herein or an anti-BCMA CAR as also set forth herein. The nucleic acid may further comprise a second nucleotide sequence encoding an armor polypeptide as disclosed herein, which enhances T cell functionality, and a third nucleotide sequence encoding a self-cleaving peptide, which is located between the first and second nucleotide sequences. In some instances, the nucleic acid is an expression vector, optionally a viral vector.

Further, provided herein is a genetically engineered immune cell, which expresses the anti-BCMA CAR and/or the anti-TACI CAR as disclosed herein, and optionally any of the armor polypeptides as also disclosed herein. In some embodiments, the genetically engineered immune cell can be a T cell, an NK cell, or a macrophage. In one example, the genetically engineered immune cell is a T cell.

In addition, the present disclosure provides a method for eliminating undesired cells in a subject, the method comprising administering to a subject in need thereof an effective amount of the genetically engineered immune cell disclosed herein, expressing any of the anti-BCMA CAR, anti-TACI CAR, and anti-BCMA/anti-TACI bispecific CARs as disclosed herein, or a pharmaceutical composition comprising such. Such genetically engineered immune cells may be armored T cells, which further express one or more armor polypeptides such as those disclosed herein.

In some embodiments, the undesired cells are cancer cells. In some embodiments, the subject is a human cancer patient. For example, the human cancer patient may comprise BCMA⁺ and/or TACI⁺ cancer cells. In some examples, the cancer cells are multiple myeloma cells, lung cancer cells, gastric cancer cells, breast cancer cells, or testis cancer cells.

Also within the scope of the present disclosure are the genetically engineered immune cells or a pharmaceutical composition comprising such as disclosed herein for use in cancer treatment. Further, provided herein are uses of the genetically engineered immune cells or a pharmaceutical composition comprising such for manufacturing a medicament for use in cancer treatment.

In addition, the present disclosure provides an anti-TACI antibody, which comprises a heavy chain variable region (V_{H}) and a light chain variable (V_{L}) region. The V_{H} and V_{L} chains comprises the same complementarity determining regions (CDRs, including CDR1, CDR2, and CDR3) as one of the reference antibodies provided in Table 2 below (e.g., TC-01). In some instances, the anti-TACI antibody comprises the same V_{H} and V_{L} as the reference antibody such as TC-01. In some examples, the anti-TACI antibody is a single-chain antibody, such as those provided in Table 2 (e.g., SEQ ID NO: 75).

Further, the present disclosure provides an anti-BCMA antibody, which comprises a heavy chain variable region (V_{H}) and a light chain variable (V_{L}) region. The V_{H} and V_{L} chains comprises the same complementarity determining regions (CDRs, including CDR1, CDR2, and CDR3) as one of the reference antibodies provided in Table 1 below (e.g., BC-06). In some instances, the anti-BCMA antibody comprises the same V_{H} and V_{L} as the reference antibody such as BC-06. In some examples, the anti-BCMA antibody is a single-chain antibody, such as those provided in Table 1 (e.g., SEQ ID NO: 50).

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

The following drawings form part of the present specification and are included to further demonstrate certain aspects of the present disclosure, which can be better understood by reference to the drawing in combination with the detailed description of specific embodiments presented herein.

FIG. 1 is a graph showing that exemplary anti-BCMA antibodies (in scFv format) as indicated specifically bind BCMA-expressing CHOK1 cells, but not the parent CHOK1 cells.

FIGS. 2A-2C include graphs showing binding of exemplary anti-BCMA antibodies (in scFv format) as indicated to cells expressing endogenous BCMA. FIG. 2A: H929 cell line. FIG. 2B: MM1.S cell line. FIG. 2C: RPM18226 cell line.

FIG. 3 is a graph showing binding activity of exemplary anti-TACI antibodies (in scFv format) to various cells as indicated.

FIGS. 4A-4E include diagrams showing cytotoxic T lymphocyte (CTL) activities of exemplar monospecific anti-BCMA CAR-T cells against various cells as indicated. FIG. 4A: CAR-T cells expressing anti-BCMA construct EPLV102 against various cell lines as indicated. FIG. 4B: CAR-T cells expressing anti-BCMA construct EPLV103 against various cell lines as indicated. FIG. 4C: CAR-T cells expressing anti-BCMA construct EPLV104 against various cell lines as indicated. FIG. 4D: CTL activity of various anti-BCMA CAR constructs as indicated against K562-GFP cells and K562-BCMA-TACI-GFP cells. FIG. 4E: secretion of IFNγ by CAR-T cells expressing various anti-BCMA CARs as indicated in the presence of target cells.

FIGS. 5A-5B include diagrams showing CTL activities of CAR-T cells expressing EPLV101, EPLV102, and EPLV103 against target cells as indicated. FIG. 5A: cell killing rates. FIG. 5B: IFNγ secretion levels.

FIGS. 6A-6E include diagrams showing CTL activities of CAR-T cells expressing EPLV102 against target cells as indicated. FIG. 6A: E:T ratio of 10:1. FIG. 6B: E:T ratio of 5:1. FIG. 6C: cell killing rates. FIG. 6D: percentages of CD4/CD8 T cell subsets. FIG. 6E: percentage of effector T cell subsets.

FIGS. 7A-7B include diagrams showing CTL activities of CAR-T cells expressing EPLV102 under target cell rechallenge. FIG. 7A: K562-GFP cells. FIG. 7B: MM1R-GFP cells.

FIGS. 8A-8E include diagrams showing CTL activities of CAR-T cells expressing EPLV200, EPLV254, EPLV255, and EPLV256 against target cells as indicated. FIG. 8A: expressing of anti-TACI CARs. FIGS. 8B-8D: CTL activities against K562-GFP cells, H929-GFP cells, and MM1R-GFP cells, respectively. FIG. 8E: cell killing rates.

FIGS. 9A-9C include diagrams showing persistency of CAR-T cells expressing anti-TACI CAR constructions with different spacers under target cell rechallenge. FIG. 9A: CAR expression levels. FIGS. 9B-9C: CTL activities against K562-GFP cells and MM1R-GFP cells, respectively.

FIGS. 10A-10B include diagrams showing expression of anti-BCMA/anti-TACI bi-specific antibodies in immune cells. FIG. 10A: bi-specific CAR construct EPLV217 expression in T cells from two donors. FIG. 10B: bi-specific CAR construct EPLV302 expression in T cells from two donors.

FIGS. 11A-11E include diagrams showing CTL activities of CAR-T cells expressing bi-specific CAR EPLV217 against various target cells. FIG. 11A: against K562-GFP cells. FIG. 11B: against H929-GFP cells. FIG. 11C: against MM1R-GFP cells. FIG. 11D: cell killing rates. FIG. 11E: IFNγ secretion.

FIGS. 12A-12I include diagrams showing CTL activities of CAR-T cells expressing bi-specific CAR EPLV302 against various target cells. FIGS. 12A-12B: CTL activity of bi-specific CAR-T cells from Donor 994 and Donor 995, respectively under K562-GFP cell rechallenge at E:T ratio of 1:1. FIGS. 12C-12D: CTL activity of bi-specific CAR-T cells from Donor 994 and Donor 995, respectively under MM1R-GFP cell rechallenge at E:T ratio of 1:1. FIGS. 12E-12F: CTL activity of bi-specific CAR-T cells from Donor 994 and Donor 995, respectively under H929-GFP cell rechallenge at E:T ratio of 1:1. FIGS. 12G-12H: cell killing rates of CAR-T cells derived from Donor 994 and Donor 995, respectively. FIG. 12I: IFNγ secretion.

FIGS. 13A-13C include diagrams showing in vivo activity of CAR-T cells expressing anti-BCMA monospecific CAR, anti-TACI monospecific CAR, and anti-BCMA/anti-TACI bi-specific CAR in an animal model. FIG. 13A: radiance levels. FIG. 13B: body weight. FIG. 13C: tumor growth.

FIGS. 14A-14E include diagrams showing bioactivities of engineered T cells expressing an anti-BCMA/TACI bispecific CAR, either alone (non-armored CAR) or in combination with an armor polypeptide (armored CAR). FIG. 14A: CAR expression levels in engineered T cells. FIG. 14B: cytotoxicity of engineered T cells against BCMA/TACI positive and BCMA/TACI negative target cells. FIG. 14C: IFNγ release at 48-hr. FIG. 14D: IFNγ release at 120-hr. FIG. 14E: IFNγ release at 168-hr.

FIGS. 15A-15B include diagrams showing anti-tumor activity of engineered T cells expressing an anti-BCMA/TACI bispecific CAR, either alone (non-armored CAR) or in combination with an armor polypeptide (armored CAR). FIG. 15A: a photo showing tumor burden at various time points after CAR-T cell treatment. FIG. 15B: a diagram showing anti-tumor activity of armored CAR-T cells relative to non-armored CAR-T cells.

FIGS. 16A-16D include diagrams showing bioactivities of engineered NK cells expressing an anti-BCMA/TACI bispecific CAR, either alone (non-armored CAR) or in combination with an armor polypeptide (armored CAR). FIG. 16A: purity of expanded NK cells. FIG. 16B: expression of CAR in transduced NK cells. FIG. 16C: cytotoxicity of engineered CAR-NK cells. FIG. 16D: IFNγ release at various time point.

### DETAILED DESCRIPTION OF THE INVENTION

B Cell Maturation Antigen (BCMA), a member of the tumor necrosis factor-receptor superfamily, is also known tumor necrosis factor receptor superfamily member 17 (TNFRSF17) and CD269. This receptor expresses at a high level on hematopoietic cancer cells, e.g., on multiple myeloma (MM) cells, but not on other normal tissues except normal plasma cells. BCMA plays a role in the regulation of B cell proliferation and survival, as well as maturation and differentiation into plasma cells.

Transmembrane activator and CAML interactor (TACI), also known as tumor necrosis factor receptor superfamily member 13B (TNFRSF13B), is a membrane protein of the TNF receptor superfamily expressed predominantly on the surface of B cells. TACI has three ligands, A proliferation-inducing ligand (APRIL), B-cell activating factor (BAFF), and Calcium modulating ligand (CAML). Binding of TACI to these ligands can trigger signaling pathways, leading to modulation of cellular activities.

Provided herein are anti-BCMA or anti-TACI monospecific chimeric antigen receptors (CARs), and anti-BCMA/anti-TACI bi-specific CARs, and CAR-T cells expressing such. In some embodiments, the CAR-T cells disclosed herein may further express an armor polypeptide, which enhances CAR-T cell features (e.g., proliferation and expansion, persistence, and/or cytotoxicity). The CAR-T cells disclosed herein may be used for treating diseases involving BCMA and/or TACI (e.g., for use in cancer therapy). The bi-specific CAR-T cells disclosed herein can activate CAR-T cells by bispecific targeting either or both of the two tumor antigens, BCMA and TAC. Such bi-specific CAR-T cells are expected to be more effective in cancer treatment, e.g., by preventing and/or treating target antigen escape. The armored CAR-T cells disclosed herein can further enhance CAR-T cell efficiency, activate immune cells, and/or mediate anti-tumor activities, overcome suppressive tumor microenvironment, thereby enhancing cancer treatment.

### I. Chimeric Antigen Receptors

As used herein, the term “chimeric antigen receptor” or “CAR” refers to an artificial immune cell receptor that is capable of binding to an antigen expressed by undesired cells, for example, an antigen of interest (TAA) (here BCMA and/or TACI). Generally, a CAR may comprise a fusion polypeptide, which comprises an extracellular antigen binding domain (e.g., a single chain variable fragment or scFv derived from an antibody specific to the target antigen), a co-stimulatory domain, and an intracellular signaling domain. In some instances, the fusion polypeptide may further comprise a hinge and transmembrane domain located at the C-terminus of the extracellular antigen binding domain. In some embodiments, the CARs disclosed herein are T cell receptors. In other embodiments, the CARs disclosed herein may be NK cell receptors.

An antibody (interchangeably used in plural form) is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term “antibody”, e.g., anti-BCMA antibody or anti-TACI antibody, encompasses not only intact (e.g., full-length) polyclonal or monoclonal antibodies, but also antigen-binding fragments thereof (such as Fab, Fab′, F(ab′)2, Fv), single-chain antibody (scFv), fusion proteins comprising an antibody portion, humanized antibodies, chimeric antibodies, diabodies, single domain antibody (e.g., nanobody), single domain antibodies (e.g., a V_{H} only antibody), multispecific antibodies (e.g., bispecific antibodies) and any other modified configuration of the immunoglobulin molecule that comprises an antigen recognition site of the required specificity, including glycosylation variants of antibodies, amino acid sequence variants of antibodies, and covalently modified antibodies. An antibody, e.g., anti-Galectin-9 antibody, includes an antibody of any class, such as IgD, IgE, IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgAQ1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known.

A typical antibody molecule comprises a heavy chain variable region (V_{H}) and a light chain variable region (V_{L}), which are usually involved in antigen binding. The V_{H} and V_{L} regions can be further subdivided into regions of hypervariability, also known as “complementarity determining regions” (“CDR”), interspersed with regions that are more conserved, which are known as “framework regions” (“FR”). Each V_{H} and V_{L} is typically composed of three CDRs and four FRs, arranged from amino-terminus to carboxy-terminus in the following order: FR1, CDR1, FR2, CDR2, FR3, CDR3, FR4. The extent of the framework region and CDRs can be precisely identified using methodology known in the art, for example, by the Kabat definition, the Chothia definition, the AbM definition, and/or the contact definition, all of which are well known in the art. See, e.g., Kabat, E. A., et al. (1991) Sequences of Proteins of Immunological Interest, Fifth Edition, U.S. Department of Health and Human Services, NIH Publication No. 91-3242. Chothia et al., (1989) Nature 342:877: Chothia, C. et al. (1987) J. Mol. Biol. 196:901-917, Al-lazikani et al (1997) J. Molec. Biol. 273:927-948; and Almagro, J. Mol. Recognit. 17:132-143 (2004). See also hgmp.mrc.ac.uk and bioinf.org.uk/abs).

In some embodiments, an antibody moiety disclosed herein may share the same heavy chain and/or light chain complementary determining regions (CDRs) or the same V_{H} and/or V_{L} chains as a reference antibody. Two antibodies having the same V_{H} and/or V_{L} CDRs means that their CDRs are identical when determined by the same approach (e.g., the Kabat approach, the Chothia approach, the AbM approach, the Contact approach, or the IMGT approach as known in the art. See, e.g., bioinf.org.uk/abs/). Such anti-BCMA or anti-TACI antibodies may have the same V_{H}, the same V_{L}, or both as compared to an exemplary antibody described herein.

In some embodiments, an antibody moiety disclosed herein may share a certain level of sequence identity as compared with a reference sequence. The “percent identity” of two amino acid sequences is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST protein searches can be performed with the XBLAST program, score=50, wordlength=3 to obtain amino acid sequences homologous to the protein molecules of interest. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

In some embodiments, an antibody moiety disclosed herein may have one or more amino acid variations relative to a reference antibody. The amino acid residue variations as disclosed in the present disclosure (e.g., in framework regions and/or in CDRs) can be conservative amino acid residue substitutions. As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g., Molecular Cloning: A Laboratory Manual, J. Sambrook, et al., eds., Second Edition, Cold Spring Harbor Laboratory Press, Cold Spring Harbor, New York, 1989, or Current Protocols in Molecular Biology, F. M. Ausubel, et al., eds., John Wiley & Sons, Inc., New York. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

The anti-BCMA monospecific CARs, anti-TACI monospecific CARs, and anti-BCMA/anti-TACI bi-specific CARs disclosed here each comprises an anti-BCMA moiety and/or an anti-TACI moiety in the extracellular antigen binding domain.

### (a) Anti-BCMA Binding Moiety

The anti-BCMA binding moiety in any of the CARs disclosed herein (e.g., any of the anti-BCMA monospecific CARs or anti-BMCA/anti-TACI bispecific CARs disclosed herein) may be in an scFv format, which is a fusion polypeptide comprising the heavy chain variable domain (V_{H}) and the light chain variable domain (V_{L}) of an anti-BCMA antibody connected by a peptide linker. In the scFv fragment, the V_{H} and V_{L} fragments may be in any orientation. In some instances, the scFv may comprise, from the N-terminus to the C-terminus, a V_{L} fragment, a peptide linker, and a V_{H} fragment. Alternatively, the scFv may comprise, from the N-terminus to the C-terminus, a V_{H} fragment, a peptide linker, and a V_{L} fragment. In some examples, a scFv may further comprise an N-terminal signal peptide for directing the CAR comprising the scFv to cell surface.

Exemplary anti-BCMA antibodies are provided in Table 1 below, any of which is within the scope of the present disclosure. In one example, the ani-BCMA antibody, e.g., for use in constructing the monospecific or bispecific CAR provided herein, may be clone BC-06.

TABLE 1
Anti-BMCA Antibodies

| | | | SEQ |
|---|---|---|---|
| Antibody | | Amino Acid Sequence* | ID |
| HC CDR1 Motif | | X₁YX₂MH, in which X₁ is S or D, and X₂ is A or G | |
| HC CDR2 Motif | | X₃IX₄YDGSX₅KYYADSVKG, in which X₃ is V or F, | 1 |
| | | X₄ is S or R, and X₅ is D or N | |
| LC CDR1 Motif A | | QASQDIX₇NYLN, in which X₇ is N or S | 2 |
| LC CDR1 Motif B | | RX₈X₉X₁₀ISSYLX₁₁, in which X₈ is A or S, X₉ is S or T, | 3 |
| | | X₁₀ is G or S, and X₁₁ is G or N | |
| LC CDR2 Motif A | | DASNX₁₂ET, in which X₁₂ is L or V | 4 |
| LC CDR2 Motif B | | AX₁₃SX₁₄LQS, in which X₁₃ is A or T, and X₁₄ is S or T | 5 |
| LC CDR3 Motif | | QQYDX₁₅LPX₁₆T, in which X₁₅ is K or N, and | 6 |
| | | X₁₆ is F, L, or Y | |
| BC-01 | V_{H} CDR1 | SYGMH | 7 |
| | V_{H} CDR2 | FIRYDGSNKYYADSVKG | 8 |
| | V_{H} CDR3 | DLWDGIVGAPAGY | 9 |
| | V_{H} | EVQLVQSGGGVVQPGGSLRLSCAASGFTFSSYGMHWVRQAPGKGLEWVAFIR | 10 |
| | | YDGSNKYYADSVKGRFTISRDNSKNTLYLQMNSLRAEDTAVYYCAKDLWDGI | |
| | | VGAPAGYWGQGTLVTVSS | |
| | V_{L} CDR1 | QASQDISNYLN | 11 |
| | V_{L} CDR2 | DASNLET | 12 |
| | V_{L} CDR3 | QQYDNLPLT | 13 |
| | V_{L} | DVVMTQSPSSLSASVGDRVTITCQASQDISNYLNWYQQKPGKAPKLLIYDAS | 14 |
| | | NLETGVPSRFSGSGSGTDFTFTISSLQPEDIATYYCQQYDNLPLTFGGGTKV | |
| | | EIK | |
| | scFv | EVQLVQSGGGVVQPGGSLRLSCAASGFTFSSYGMHWVRQAPGKGLEWVAFIR | 15 |
| | | YDGSNKYYADSVKGRFTISRDNSKNTLYLQMNSLRAEDTAVYYCAKDLWDGI | |
| | | VGAPAGYWGQGTLVTVSSGGGGSGGGGSGGGGSDVVMTQSPSSLSASVGDRV | |
| | | TITCQASQDISNYLNWYQQKPGKAPKLLIYDASNLETGVPSRFSGSGSGTDF | |
| | | TFTISSLQPEDIATYYCQQYDNLPLTFGGGTKVEIK | |
| BC-02 | V_{H} CDR1 | SYGMH | 7 |
| | V_{H} CDR2 | FIRYDGSNKYYADSVKG | 8 |
| | V_{H} CDR3 | DWDYYDSSGYYPDALGI | 16 |
| | V_{H} | QVQLVQSGGGVVQPGGSLRLSCAASGFTFSSYGMHWVRQAPGKVLEWVAFIR | 17 |
| | | YDGSNKYYADSVKGRFTISRDNSKNTLYLQMNSLRAEDTAVYYCAKDWDYYD | |
| | | SSGYYPDALGIWGQGTMVTVSS | |
| | V_{L} CDR1 | QASQDISNYLN | 11 |
| | V_{L} CDR2 | DASNLET | 12 |
| | V_{L} CDR3 | QQYDNLPFT | 18 |
| | V_{L} | EIVLTQSPSSLSASVGDRVTITCQASQDISNYLNWYQQKPGKAPKLLIYDAS | 19 |
| | | NLETGVPSRFSGSGSGTDFTFTISSLQPEDIATYYCQQYDNLPFTFGPGTKV | |
| | | DIK | |
| | scFv | QVQLVQSGGGVVQPGGSLRLSCAASGFTFSSYGMHWVRQAPGKVLEWVAFIR | 20 |
| | | YDGSNKYYADSVKGRFTISRDNSKNTLYLQMNSLRAEDTAVYYCAKDWDYYD | |
| | | SSGYYPDALGIWGQGTMVTVSSGGGGSGGGGSGGGGSEIVLTQSPSSLSASV | |
| | | GDRVTITCQASQDISNYLNWYQQKPGKAPKLLIYDASNLETGVPSRFSGSGS | |
| | | GTDFTFTISSLQPEDIATYYCQQYDNLPFTFGPGTKVDIK | |
| BC-03 | V_{H} CDR1 | SYGMH | 7 |
| | V_{H} CDR2 | FIRYDGSDKYYADSVKG | 21 |
| | V_{H} CDR3 | DLTTITPGY | 22 |
| | V_{H} | QMQLVQSGGGVVQPGGSLRLSCAASGFTFSSYGMHWVRQAPGKGLEWVAFIR | 23 |
| | | YDGSDKYYADSVKGRFTISRDNSKNTLYLQMNSLRAEDTAVYYCAKDLTTIT | |
| | | PGYWGQGTLVTVSS | |
| | V_{L} CDR1 | QASQDISNYLN | 11 |
| | V_{L} CDR2 | DASNLET | 12 |
| | V_{L} CDR3 | QQYDNLPLT | 13 |
| | V_{L} | VIQMTQSPSSLSASVGDRVTITCQASQDISNYLNWYQQKPGKAPKLLIYDAS | 24 |
| | | NLETGVPSRFSGSGSGTDFTFTISSLQPEDIATYYCQQYDNLPLTFGGGTKV | |
| | | EIK | |
| | scFv | QMQLVQSGGGVVQPGGSLRLSCAASGFTFSSYGMHWVRQAPGKGLEWVAFIR | 25 |
| | | YDGSDKYYADSVKGRFTISRDNSKNTLYLQMNSLRAEDTAVYYCAKDLTTIT | |
| | | PGYWGQGTLVTVSSGGGGSGGGGSGGGGSVIQMTQSPSSLSASVGDRVTITC | |
| | | QASQDISNYLNWYQQKPGKAPKLLIYDASNLETGVPSRFSGSGSGTDFTFTI | |
| | | SSLQPEDIATYYCQQYDNLPLTFGGGTKVEIK | |
| BC-04 | V_{H} CDR1 | DYAMS | 26 |
| | V_{H} CDR2 | FIRSKAYGGTTEYAASVKG | 27 |
| | V_{H} CDR3 | GPHYDILTSNWFDP | 28 |
| | V_{H} | EVQLVESGGGLVQPGRSLRLSCTASGFTFGDYAMSWFRQAPGKGLEWVGFIR | 29 |
| | | SKAYGGTTEYAASVKGRFTISRDDSKSIAYLQMNSLKTEDTAVYYCTPGPHY | |
| | | DILTSNWFDPWGQGTLVTVSS | |
| | V_{L} CDR1 | RASQSISSYLN | 30 |
| | V_{L} CDR2 | AASSLQS | 31 |
| | V_{L} CDR3 | QHSYSTPHT | 32 |
| | V_{L} | DIVMTHSPSSLSASVGDRVTITCRASQSISSYLNWYQQKPGKAPKLLIYAAS | 33 |
| | | SLQSGVPSRFSGSGSGTDFTLTISSLQPEDFATYYCQHSYSTPHTFGQGTKV | |
| | | EIK | |
| | scFv | EVQLVESGGGLVQPGRSLRLSCTASGFTFGDYAMSWFRQAPGKGLEWVGFIR | 34 |
| | | SKAYGGTTEYAASVKGRFTISRDDSKSIAYLQMNSLKTEDTAVYYCTPGPHY | |
| | | DILTSNWFDPWGQGTLVTVSSGGGGSGGGGSGGGGSDIVMTHSPSSLSASVG | |
| | | DRVTITCRASQSISSYLNWYQQKPGKAPKLLIYAASSLQSGVPSRFSGSGSG | |
| | | TDFTLTISSLQPEDFATYYCQHSYSTPHTFGQGTKVEIK | |
| BC-05 | V_{H} CDR1 | SYGMH | 7 |
| | V_{H} CDR2 | FIRYDGSNKYYADSVKG | 8 |
| | V_{H} CDR3 | DWEDPLYYYDTPF | 35 |
| | V_{H} | QMQLVQSGGGVVQPGGSLRLSCAASGFTFSSYGMHWVRQAPGKGLEWVAFIR | 36 |
| | | YDGSNKYYADSVKGRFTISRDNSKNTLYLQMNSLRAEDTAVYYCAKDWEDPL | |
| | | YYYDTPFWGQGTLVTVSS | |
| | V_{L} CDR1 | QASQDINNYLN | 37 |
| | V_{L} CDR2 | DASNVET | 38 |
| | V_{L} CDR3 | QQYDKLPYT | 39 |
| | V_{L} | VIWMTQSPSSVSASVGDIITITCQASQDINNYLNWYQLKPGKAPKLLIYDAS | 40 |
| | | NVETGVPSRFSGSGSGTDFTFTISSLQPEDTATYYCQQYDKLPYTFGQGTKL | |
| | | EIK | |
| | scFv | QMQLVQSGGGVVQPGGSLRLSCAASGFTFSSYGMHWVRQAPGKGLEWVAFIR | 41 |
| | | YDGSNKYYADSVKGRFTISRDNSKNTLYLQMNSLRAEDTAVYYCAKDWEDPL | |
| | | YYYDTPFWGQGTLVTVSSGGGGSGGGGSGGGGSVIWMTQSPSSVSASVGDII | |
| | | TITCQASQDINNYLNWYQLKPGKAPKLLIYDASNVETGVPSRFSGSGSGTDF | |
| | | TFTISSLQPEDTATYYCQQYDKLPYTFGQGTKLEIK | |
| BC-06 | V_{H} CDR1 | DYAMH | 42 |
| | V_{H} CDR2 | GISWNSGSIGYADSVKG | 43 |
| | V_{H} CDR3 | VQPPGAFDI | 44 |
| | V_{H} | EVQLVESGGGLVQPGRSLRLSCAASGFTFDDYAMHWVRQAPGKGLEWVSGIS | 45 |
| | | WNSGSIGYADSVKGRFTISRDNAKNSLYLQMNSLRAEDTALYYCAKVQPPGA | |
| | | FDIWGQGTMVTVSS | |
| | V_{L} CDR1 | RSTQGISSYLG | 46 |
| | V_{L} CDR2 | ATSTLQS | 47 |
| | V_{L} CDR3 | QQLYS | 48 |
| | V_{L} | DIQLTQSPSFLSASVGDRVSITCRSTQGISSYLGWYQQKPGKAPKLLIYATS | 49 |
| | | TLQSGVPSRFSGSGSGTEFTLTISSLQPEDFATYYCQQLYSFGQGTKLEIK | |
| | scFv | EVQLVESGGGLVQPGRSLRLSCAASGFTFDDYAMHWVRQAPGKGLEWVSGIS | 50 |
| | | WNSGSIGYADSVKGRFTISRDNAKNSLYLQMNSLRAEDTALYYCAKVQPPGA | |
| | | FDIWGQGTMVTVSSGGGGSGGGGSGGGGSDIQLTQSPSFLSASVGDRVSITC | |
| | | RSTQGISSYLGWYQQKPGKAPKLLIYATSTLQSGVPSRFSGSGSGTEFTLTI | |
| | | SSLQPEDFATYYCQQLYSFGQGTKLEIK | |
| BC-07 | V_{H} CDR1 | DYAMH | 42 |
| | V_{H} CDR2 | GISWNSGSIGYADSVKG | 43 |
| | V_{H} CDR3 | VQSPGAFDI | 51 |
| | V_{H} | EVQLVESGGGLVQPGRSLRLSCAASGFTFDDYAMHWVRQAPGKGLEWVSGIS | 52 |
| | | WNSGSIGYADSVKGRFTISRDNAKNSLYLQMNSLRAEDTALYYCAKVQSPGA | |
| | | FDIWGQGTMVTVSS | |
| | V_{L} CDR1 | RSTQGISSYLG | 46 |
| | V_{L} CDR2 | ATSTLQS | 47 |
| | V_{L} CDR3 | QQLYS | 48 |
| | V_{L} | DIQLTQSPSFLSASVGDRVSITCRSTQGISSYLGWYQQKPGKAPKLLIYATS | 49 |
| | | TLQSGVPSRFSGSGSGTEFTLTISSLQPEDFATYYCQQLYSFGQGTKLEIK | |
| | scFv | EVQLVESGGGLVQPGRSLRLSCAASGFTFDDYAMHWVRQAPGKGLEWVSGIS | 53 |
| | | WNSGSIGYADSVKGRFTISRDNAKNSLYLQMNSLRAEDTALYYCAKVQSPGA | |
| | | FDIWGQGTMVTVSSGGGGSGGGGSGGGGSDIQLTQSPSFLSASVGDRVSITC | |
| | | RSTQGISSYLGWYQQKPGKAPKLLIYATSTLQSGVPSRFSGSGSGTEFTLTI | |
| | | SSLQPEDFATYYCQQLYSFGQGTKLEIK | |
| BC-08 | V_{H} CDR1 | SYAMH | 54 |
| | V_{H} CDR2 | VISYDGSNKYYADSVKG | 55 |
| | V_{H} CDR3 | DEHQVVPNYRFDF | 56 |
| | V_{H} | QVQLVQSGGGVVQPGRSLRLSCAASGFTFSSYAMHWVRQAPGKGLEWVAVIS | 57 |
| | | YDGSNKYYADSVKGRFTISRDNAENSVYLQMNSLRAEDTAVYYCARDEHQVV | |
| | | PNYRFDFWGQGSQVTVSS | |
| | V_{L} CDR1 | SGSGSNIGSNDVS | 58 |
| | V_{L} CDR2 | WNDQRPS | 59 |
| | V_{L} CDR3 | AAWDDSLNGWV | 60 |
| | V_{L} | QLVLTQPPSASGTPGQRVTISCSGSGSNIGSNDVSWYQQIPGTASKLLIYWN | 61 |
| | | DQRPSGVPDRFSGSKSGTSASLAISGLQSEDEADYYCAAWDDSLNGWVFGGG | |
| | | TKLTVL | |
| | scFv | QVQLVQSGGGVVQPGRSLRLSCAASGFTFSSYAMHWVRQAPGKGLEWVAVIS | 62 |
| | | YDGSNKYYADSVKGRFTISRDNAENSVYLQMNSLRAEDTAVYYCARDEHQVV | |
| | | PNYRFDFWGQGSQVTVSSGGGGSGGGGSGGGGSQLVLTQPPSASGTPGQRVT | |
| | | ISCSGSGSNIGSNDVSWYQQIPGTASKLLIYWNDQRPSGVPDRFSGSKSGTS | |
| | | ASLAISGLQSEDEADYYCAAWDDSLNGWVFGGGTKLTVL | |
| BC-09 | V_{H} CDR1 | SYGMH | 7 |
| | V_{H} CDR2 | FIRYDGSNKYYADSVKG | 8 |
| | V_{H} CDR3 | DLWEFGGDYADY | 63 |
| | V_{H} | QVQLVQSGGGVVQPGGSLRLSCAASGFTFSSYGMHWVRQAPGKGLEWVAFIR | 64 |
| | | YDGSNKYYADSVKGRFTISRDNSKNTLYLQMNSLRAEDTAVYYCAKDLWEFG | |
| | | GDYADYWGQGTLVTVSS | |
| | V_{L} CDR1 | QASQDISNYLN | 11 |
| | V_{L} CDR2 | DASNLET | 12 |
| | V_{L} CDR3 | QQYDNLPLT | 13 |
| | V_{L} | NIQMTQSPSSLSASVGDRVTITCQASQDISNYLNWYQQKPGKAPKLLIYDAS | 65 |
| | | NLETGVPSRFSGSGSGTDFTFTISSLQPEDIATYYCQQYDNLPLTFGGGTKV | |
| | | EIK | |
| | scFv | QVQLVQSGGGVVQPGGSLRLSCAASGFTFSSYGMHWVRQAPGKGLEWVAFIR | 66 |
| | | YDGSNKYYADSVKGRFTISRDNSKNTLYLQMNSLRAEDTAVYYCAKDLWEFG | |
| | | GDYADYWGQGTLVTVSSGGGGSGGGGSGGGGSNIQMTQSPSSLSASVGDRVT | |
| | | ITCQASQDISNYLNWYQQKPGKAPKLLIYDASNLETGVPSRFSGSGSGTDFT | |
| | | FTISSLQPEDIATYYCQQYDNLPLTFGGGTKVEIK | |

* CDRs determined by the Chothia scheme

An anti-BCMA binding moiety (and an anti-TACI binding moiety disclosed below) derived from a reference antibody refers to binding moieties having substantially similar structural and functional features as the reference antibody. Structurally, the binding moiety may have the same heavy and/or light chain complementary determining regions or the same V_{H} and/or V_{L} chains as the reference antibody. Alternatively, the binding moiety may only have a limited number of amino acid variations in one or more of the framework regions and/or in one or more of the CDRs without significantly affecting its binding affinity and binding specificity relative to the reference antibody. See descriptions below.

In some embodiments, the anti-BCMA binding moiety may comprise one or more of the heavy chain and light chain complementary determining region (CDR) motifs provided in Table 1 above, for example, comprising all of the six CDR motifs provided in Table 1. In some examples, the ani-BCMA binding moiety may be derived from anti-BCMA antibody BC-05. In some examples, the anti-BCMA binding moiety may be derived from anti-BCMA antibody BC-06. In some examples, the anti-BCMA binding moiety may be derived from anti-BCMA antibody BC-08.

In some examples, the anti-BCMA binding moiety may comprise the same heavy chain CDRs as those in any of the reference antibodies provided in Table 1 above (e.g., BC-05, BC-06, or BC-08). Alternatively, or in addition, the anti-BCMA binding moiety may have the same light chain CDRs as those in any of the reference antibodies provided in Table 1 above (e.g., BC-05, BC-06, or BC-08). Such an anti-BCMA binding moiety may comprise the same V_{H} and/or V_{L} chains as the reference antibody. Alternatively, the anti-BCMA binding moiety may comprise amino acid variations in one or more of the framework regions relative to the corresponding framework regions in the reference antibody. For example, the anti-BCMA binding moiety may comprise, collectively, up to 15 amino acid variations (e.g., up to 12, 10, 9, 8, 7, 6, 5, 4, 3, 2, or 1 amino acid variations) in one or more framework regions relative to the corresponding framework regions in the reference antibody.

In some embodiments, the anti-BCMA moiety may comprise a certain level of variations in one or more of the CDRs relative to those in any of the reference antibodies provided in Table 1 above (e.g., BC-05, BC-06, or BC-08). For example, the anti-BCMA moiety may comprise heavy chain CDRs that are at least 80% (e.g., 85%, 90%, 95%, or 98%) sequence identity, individually or collectively, as compared with the V_{H} CDRs of the reference antibody. Alternatively, or in addition, the anti-BCMA antibody may comprise light chain CDRs that are at least 80% (e.g., 85%, 90%, 95%, or 98%) sequence identity, individually or collectively, as compared with the V_{L} CDRs as the reference antibody. As used herein, “individually” means that one CDR of an antibody shares the indicated sequence identity relative to the corresponding CDR of a reference antibody (e.g., those in Table 1 such as BC-05, BC-06, or BC-08 or any of the anti-TACI reference antibodies disclosed below). “Collectively” means that three V_{H} or V_{L} CDRs of an antibody in combination share the indicated sequence identity relative the corresponding three V_{H} or V_{L} CDRs of the reference antibody in combination.

In some instances, the anti-BCMA moiety may comprise up to 10 amino acid variations (e.g., up to 9, 8, 7. 6, 5, 4, 3, 2, or 1 amino acid variations) in one or more of the heavy chain and light chain CDRs collectively relative to those in the CDRs of a reference antibody provided in Table 1 (e.g., BC-05, BC-06, or BC-08). In some instances, the anti-BCMA moiety may comprise the same heavy chain CDR3 as the heavy chain CDR3 of the reference antibody and comprise one or more amino acid variations in one or more of the other heavy chain and light chain CDRs.

In some examples, the anti-BCMA moiety disclosed herein may be any of the anti-BCMA scFv fragments provided in Table 1 above. In specific examples, the anti-BCMA scFv may comprise the amino acid sequence of SEQ ID NO: 41, 50, or 62. Alternatively, the anti-BCMA moiety may comprise an amino acid sequence at least 85% (e.g., at least 90%, at least 95%, at least 98%, or above) identical to those provided in Table 1, e.g., SEQ ID NO: 41, 50, or 62. In other examples, the anti-BCMA moiety disclosed herein may comprise the same V_{H} and V_{L} sequences as in those listed in Table 1, e.g., SEQ ID NO: 41, 50, or 62, but has a reversed orientation of the V_{H} and V_{L} fragments.

Any of the anti-BCMA moieties disclosed herein (e.g., those provided in Table 1 such as SEQ ID NO: 41, 50, or 62 or its counterpart having reversed V_{H} and V_{L} orientation) may be used for constructing anti-BCMA monospecific CAR constructs and/or anti-BCMA/anti-TACI bi-specific CAR constructs as disclosed herein.

### (b) Anti-TACI Binding Moiety

The anti-TACI binding moiety in any of the CARs disclosed herein (e.g., any of the anti-TACI monospecific CARs and/or anti-BCMA/anti-TACI bispecific CARs disclosed herein) may be in an scFv format, which is a fusion polypeptide comprising the heavy chain variable domain (V_{H}) and the light chain variable domain (V_{L}) of an anti-TACI antibody connected by a peptide linker. In the scFv fragment, the V_{H} and V_{L} fragments may be in any orientation. In some instances, the scFv may comprise, from the N-terminus to the C-terminus, a V_{L} fragment, a peptide linker, and a V_{H} fragment. Alternatively, the scFv may comprise, from the N-terminus to the C-terminus, a V_{H} fragment, a peptide linker, and a V_{L} fragment. In some examples, a scFv may further comprise an N-terminal signal peptide for directing the CAR comprising the scFv to cell surface.

In some embodiments, the anti-TACI binding moiety may be derived from any of those provided in Table 2 below. The heavy chain and light chain complementary determining regions provided in Table 2 (as well as in Table 1) are based on Chothia definition.

TABLE 2
Anti-TACI Antibodies

| | | | SEQ |
|---|---|---|---|
| Antibody | | Amino Acid Sequence | ID |
| TC-01 | V_{H} CDR1 | GYFMH | 67 |
| | V_{H} CDR2 | WINSNSGGTNYAQKFQG | 68 |
| | V_{H} CDR3 | DDGTGYNAFDI | 69 |
| | V_{H} | QVQLVQSGAEVKKPGASVKVSCKASGYTFTGYFMHWVRQAPGQGLEWMGWINS | 70 |
| | | NSGGTNYAQKFQGRVTMTRDTSISTAYMELSRLRSDDTAVYYCARDDGTGYNA | |
| | | FDIWGQGTMVTVSS | |
| | V_{L} CDR1 | RSSQSLLHSNGYNYLD | 71 |
| | V_{L} CDR2 | LASHRAS | 72 |
| | V_{L} CDR3 | MQAQQTAL | 73 |
| | V_{L} | DIVMTQSPLSLPVTPGEPASISCRSSQSLLHSNGYNYLDWYVQKPGKPPQLLI | 74 |
| | | YLASHRASGVPDRFSGSGSGTDFTLKISRVEAEDVGIYYCMQAQQTALFGPGT | |
| | | KVDIK | |
| | scFv | QVQLVQSGAEVKKPGASVKVSCKASGYTFTGYFMHWVRQAPGQGLEWMGWINS | 75 |
| | | NSGGTNYAQKFQGRVTMTRDTSISTAYMELSRLRSDDTAVYYCARDDGTGYNA | |
| | | FDIWGQGTMVTVSSGGGGSGGGGSGGGGSDIVMTQSPLSLPVTPGEPASISCR | |
| | | SSQSLLHSNGYNYLDWYVQKPGKPPQLLIYLASHRASGVPDRFSGSGSGTDFT | |
| | | LKISRVEAEDVGIYYCMQAQQTALFGPGTKVDIK | |
| TC-02 | V_{H} CDR1 | GYYWS | 76 |
| | V_{H} CDR2 | EINHSGSTNYNPSLKS | 77 |
| | V_{H} CDR3 | IRKLGGYYFDY | 78 |
| | V_{H} | QVQLQQWGAGLLKPSETLSLTCAVYGGSFSGYYWSWIRQPPGKGLEWIGEINH | 79 |
| | | SGSTNYNPSLKSRVTISVDTSKNQFSLKLSSVTAADTAVYYCARIRKLGGYYF | |
| | | DYWGQGTLVTVSS | |
| | V_{L} CDR1 | RASQSVSSSYLA | 80 |
| | V_{L} CDR2 | GASSRAT | 81 |
| | V_{L} CDR3 | QQYGSSPRP | 82 |
| | V_{L} | EIVMTQSPGTLSLSPGERATLSCRASQSVSSSYLAWYQQKPGQAPRLLIYGAS | 83 |
| | | SRATGIPDRFSGSGSGTDFTLTISRLEPEDFAVYYCQQYGSSPRPFGQGTKVE | |
| | | IK | |
| | scFv | QVQLQQWGAGLLKPSETLSLTCAVYGGSFSGYYWSWIRQPPGKGLEWIGEINH | 84 |
| | | SGSTNYNPSLKSRVTISVDTSKNQFSLKLSSVTAADTAVYYCARIRKLGGYYF | |
| | | DYWGQGTLVTVSSGGGGSGGGGSGGGGSEIVMTQSPGTLSLSPGERATLSCRA | |
| | | SQSVSSSYLAWYQQKPGQAPRLLIYGASSRATGIPDRFSGSGSGTDFTLTISR | |
| | | LEPEDFAVYYCQQYGSSPRPFGQGTKVEIK | |
| TC-03 | V_{H} CDR1 | SDYMS | 85 |
| | V_{H} CDR2 | FIYRGGSTYYADSVKG | 86 |
| | V_{H} CDR3 | GLGSSSDY | 87 |
| | V_{H} | EVQLVESGGGLVQPGGSLRLSCATSGFTVNSDYMSWVRQSPEKGLEWVSFIYR | 88 |
| | | GGSTYYADSVKGRFTVSRDTSKNTLYLQMNSLRVEDTAVYYCARGLGSSSDYW | |
| | | GQGTLVTVSS | |
| | V_{L} CDR1 | TRSSGSIANNYVQ | 89 |
| | V_{L} CDR2 | EDDHRPS | 90 |
| | V_{L} CDR3 | QSYDGIDHYV | 91 |
| | V_{L} | SYELTQPHSVSESPGKTVTISCTRSSGSIANNYVQWYRHRPGSAPSTVIFEDD | 92 |
| | | HRPSEVPDRFSGSIDSSSNSASLTISGLKTEDEADYYCQSYDGIDHYVFGTGT | |
| | | KLTVL | |
| | scFv | EVQLVESGGGLVQPGGSLRLSCATSGFTVNSDYMSWVRQSPEKGLEWVSFIYR | 93 |
| | | GGSTYYADSVKGRFTVSRDTSKNTLYLQMNSLRVEDTAVYYCARGLGSSSDYW | |
| | | GQGTLVTVSSGGGGSGGGGSGGGGSSYELTQPHSVSESPGKTVTISCTRSSGS | |
| | | IANNYVQWYRHRPGSAPSTVIFEDDHRPSEVPDRFSGSIDSSSNSASLTISGL | |
| | | KTEDEADYYCQSYDGIDHYVFGTGTKLTVL | |
| TC-04 | V_{H} CDR1 | SYAIS | 94 |
| | V_{H} CDR2 | GIIPIFGIANYAQKFQG | 95 |
| | V_{H} CDR3 | DKANTALRWFDP | 96 |
| | V_{H} | EVQLVQSGAEVKKPGSSVKVSCKASGGTFSSYAISWVRQAPGQGLEWMGGIIP | 97 |
| | | IFGTANYAQKFQGRVTITADKSTSTAYMELSSLRSEDTAVYYCARDKANTALR | |
| | | WFDPWGQGTLVTVSS | |
| | V_{L} CDR1 | RASQRVSSSYLA | 98 |
| | V_{L} CDR2 | GASKRAT | 99 |
| | V_{L} CDR3 | QQYGDSPLT | 100 |
| | V_{L} | EIVMTQSPGSLSLSPGDTATLSCRASQRVSSSYLAWYQQKPGQAPRLLIYGAS | 101 |
| | | KRATGISSKFSGSGSETDFLLTISRLEPEDIAVYYCQQYGDSPLTFGQGTKVE | |
| | | IK | |
| | scFv | EVQLVQSGAEVKKPGSSVKVSCKASGGTFSSYAISWVRQAPGQGLEWMGGIIP | 102 |
| | | IFGTANYAQKFQGRVTITADKSTSTAYMELSSLRSEDTAVYYCARDKANTALR | |
| | | WFDPWGQGTLVTVSSGGGGSGGGGSGGGGSEIVMTQSPGSLSLSPGDTATLSC | |
| | | RASQRVSSSYLAWYQQKPGQAPRLLIYGASKRATGISSKFSGSGSETDFLLTI | |
| | | SRLEPEDIAVYYCQQYGDSPLTFGQGTKVEIK | |

In some examples, the anti-TACI binding moiety may comprise the same heavy chain CDRs as those in any of the reference antibodies listed in Table 2 above, e.g., TC-01. Alternatively, or in addition, the anti-TACI binding moiety may have the same light chain CDRs as those in the reference antibody, e.g., TC-01. Such an anti-TACI binding moiety may comprise the same V_{H} and/or V_{L} chains as the reference antibody. Alternatively, the anti-TACI binding moiety may comprise amino acid variations in one or more of the framework regions relative to the corresponding framework regions in the reference antibody. For example, the anti-TACI binding moiety may comprise, collectively, up to 15 amino acid variations (e.g., up to 12, 10, 9, 8, 7, 6, 5, 4, 3, 2, or 1 amino acid variations) in one or more framework regions relative to the corresponding framework regions in the reference antibody.

In some embodiments, the anti-TACI binding moiety may comprise a certain level of variations in one or more of the CDRs relative to those of a reference antibody provided in Table 2 above (e.g., TC-01). For example, the anti-TACI binding moiety may comprise heavy chain CDRs that are at least 80% (e.g., 85%, 90%, 95%, or 98%) sequence identity, individually or collectively, as compared with the V_{H} CDRs of the reference antibody. Alternatively, or in addition, the anti-TACI antibody may comprise light chain CDRs that are at least 80% (e.g., 85%, 90%, 95%, or 98%) sequence identity, individually or collectively, as compared with the V_{L} CDRs as the reference antibody.

In some instances, the anti-TACI binding moiety may comprise up to 10 amino acid variations (e.g., up to 9, 8, 7. 6, 5, 4, 3, 2, or 1 amino acid variations) in one or more of the heavy chain and light chain CDRs collectively relative to those in the CDRs of a reference antibody provided in Table 2 (e.g., TC-01). In some instances, the anti-TACI binding moiety may comprise the same heavy chain CDR3 as the heavy chain CDR3 of the reference antibody and comprise one or more amino acid variations in one or more of the other heavy chain and light chain CDRs.

In some examples, the anti-TACI binding moiety disclosed herein may comprise an amino acid sequence of any of the anti-TACI scFv fragments provided in Table 2 above (e.g., SEQ ID NO: 75). Alternatively, the anti-TACI moiety may comprise an amino acid sequence at least 85% (e.g., at least 90%, at least 95%, at least 98%, or above) identical to that of a reference antibody provided in Table 2 (e.g., SEQ ID NO: 75). In other examples, the anti-TACI binding moiety disclosed herein may comprise the same V_{H} and V_{L} sequences as in that of a reference antibody (e.g., SEQ ID NO: 75) but has a reversed orientation of the V_{H} and V_{L} fragments as in the reference antibody.

Any of the anti-TACI binding moieties disclosed herein (e.g., those provided in Table 2) may be used for constructing the anti-TACI monospecific and/or anti-BCMA/anti-TACI bispecific CARs as disclosed herein. In some examples, the anti-TACI moiety may comprise the amino acid sequence of SEQ ID NO: 75, or its counterpart having reversed V_{H} and V_{L} orientation.

### (c) Other Components of Chimeric Antigen Receptor Constructs

In addition to the extracellular antigen binding domains disclosed herein, any of the CARs, including the anti-BCMA CAR, the anti-TACI CAR, or the anti-BCMA/anti-TACI bispecific CARs, may further comprise one or more intracellular signaling domains (e.g., co-stimulatory and cytoplasmic signaling domains), and optionally a hinge domain, a transmembrane domain, an N-terminal signal peptide, or a combination thereof. In some instances, the CAR can be co-expressed with an armor polypeptide in a host immune cell, which enhances physical and/or biological features of the host immune cells. See, e.g., disclosures herein. For example, the CAR coding sequence and the suicide gene may be configured in a bicistronic expression cassette, in which the CAR coding sequence and the armor gene may be linked via a self-cleavage peptide (e.g., P2A or T2A) coding sequence. Examples are provided in Table 3 below.

TABLE 3
Exemplary Components of
Chimeric Antigen Receptor Constructs.

| CAR | | SEQ |
|---|---|---|
| Components | Amino Acid Sequence | ID |
| Signal peptide | MLLLVTSLLLCELPHPAFLLIP | 103 |
| Linker between | GGGGS | 104 |
| two scFvs | | |
| Linker between | GGGGSGGGGS | 105 |
| two scFvs | | |
| Linker between | GGGGSGGGGSGGGGS | 106 |
| two scFvs | | |
| Linker between | GSTSGSGKPGSGEGSTKG | 107 |
| two scFvs | | |
| Flag tag | DYKDDDDK | 108 |
| Hinge | ESKYGPPCPPCP | 109 |
| Spacer-1 | GQPREPQVYTLPPSQEEMTKNQ | 110 |
| | VSLTCLVKGFYPSDIAVEWESN | |
| | GQPENNYKTTPPVLDSDGSFFL | |
| | YSKLTVDKSRWQEGNVFSCSVM | |
| | HEALHNHYTQESLSLSP | |
| Spacer-2 | APEFEGAPSVFLFPPKPKDTLM | 111 |
| | ISRTPEVTCVVVDVSQEDPEVQ | |
| | FNWYVDGVEVHNAKTKPREEQF | |
| | QSTYRVVSVLTVLHQDWLNGKE | |
| | YKCKVSNKGLPSSIEKTISKAK | |
| | GQPREPQVYTLPPSQEEMTKNQ | |
| | VSLTCLVKGFYPSDIAVEWESN | |
| | GQPENNYKTTPPVLDSDGSFFL | |
| | YSKLTVDKSRWQEGNVFSCSVM | |
| | HEALHNHYTQESLSLSP | |
| CD8 | MFWVLVVVGGVLACYSLLVTVA | 112 |
| Transmembrane | FIIFWV | |
| (TM) | | |
| T2A | LEGGGEGRGSLLTCGDVEENPG | 113 |
| | PR | |
| CD16a TM | VSFCLVMVLLFAVDTGLYFSV | 114 |
| CD28 TM | FWVLVVVGGVLACYSLLVTVAF | 115 |
| | IIFWV | |
| 41BB Co- | KRGRKKLLYIFKQPFMRPVQTT | 116 |
| stimulatory | QEEDGCSCRFPEEEEGGCEL | |
| signaling (CoS) | | |
| CD28 CoS-Mut | RSKRSRLLHSDYMFMTPRRPGP | 117 |
| | TRKHYQPYAPPRDFAAYRS | |
| CD3ζ | RVKFSRSADAPAYQQGQNQLYN | 118 |
| | ELNLGRREEYDVLDKRRGRDPE | |
| | MGGKPRRKNPQEGLYNELQKDK | |
| | MAEAYSEIGMKGERRRGKGHDG | |
| | LYQGLSTATKDTYDALHMQALP | |
| | PR | |

### Signaling Domains

Any of the CAR constructs disclosed herein, including anti-BCMA CAR, anti-TACI CAR, or anti-BCMA/anti-TACI bispecific CARs, comprise one or more intracellular signaling domains, which typically contain a co-stimulatory domain and a cytoplasmic signaling domain. A “co-stimulatory signaling domain” refers to at least a fragment of a co-stimulatory signaling protein that mediates signal transduction within a cell to induce an immune response such as an effector function (a secondary signal). A cytoplasmic signaling domain may be any signaling domain involved in triggering cell signaling (primary signaling) that leads to immune cell proliferation and/or activation. The cytoplasmic signaling domain as described herein is not a co-stimulatory signaling domain, which, as known in the art, relays a co-stimulatory or secondary signal for fully activating immune cells.

In some embodiments, the co-stimulatory signaling domain and the cytoplasmic signaling domain are for use in CAR constructs disclosed herein that are to be introduced into T cells. In some instances, a co-stimulatory signaling domain may be derived from a co-stimulatory protein involved in T cell responses, for example, a member of the B7/CD28 family, a member of the TNF superfamily, a member of the SLAM family, or any other co-stimulatory molecules. Examples include, but are not limited to, 4-1BB, CD28, OX40, ICOS, CD40, CD40L, CD27, GITR, HVEM, TIM1, LFA1(CD11a) or CD2. In specific examples, the co-stimulatory signaling domain is a 4-1BB signaling domain (e.g., SEQ ID NO: 116 in Table 3 above). In other specific examples, the co-stimulatory signaling domain is a CD28 signaling domain (e.g., SEQ ID NO: 117 in Table 3 above).

The cytoplasmic signaling domain may comprise an immunoreceptor tyrosine-based activation motif (ITAM) domain or may be ITAM free. An “ITAM,” as used herein, is a conserved protein motif that is generally present in the tail portion of signaling molecules expressed in many immune cells. Exemplary cytoplasmic signaling domains include the signaling domain of CD3ζ, e.g., SEQ ID NO: 118.

### Hinge and Transmembrane Domains

In some instances, the CAR construct disclosed herein (e.g., any of the anti-BCMA CARs, anti-TACI CARs, or anti-BCMA/anti-TACI bispecific CARs disclosed herein) may contain a transmembrane domain, which can be a hydrophobic alpha helix that spans the membrane. A “transmembrane domain” can be a peptide fragment that is thermodynamically stable in a cell membrane, preferably a eukaryotic cell membrane. The transmembrane domain can provide stability of the CAR containing such. Exemplary transmembrane domains may be a CD8 transmembrane domain, or a CD28 transmembrane domain. In one example, the transmembrane domain can comprise SEQ ID NO: 109, 110, 111, or 112 shown in Table 3 above.

Alternatively, or in addition, the CAR construct disclosed herein may also comprise a hinge domain, which may be located between the extracellular antigen binding domain and the transmembrane domain, or between the transmembrane domain and the intracellular signaling domain. A hinge domain may function to provide flexibility to the CAR, or domains thereof, or to prevent steric hindrance of the CAR, or domains thereof. A hinge domain may contain 5-20 amino acid residues. In some embodiments, the hinge domain may be a CD8 hinge domain or an IgG hinge. Other hinge domains may be used. In one example, the hinge domain can comprise SEQ ID NO: 106 shown in Table 3 above.

### (d) Anti-BCMA/Anti-TACI Bi-Specific CARs

In some aspects, provided herein are anti-BCMA/anti-TACI bispecific CARs each comprising an anti-BCMA binding moiety (e.g., an anti-BCMA scFv such as those disclosed herein; see Table 1 above, such as SEQ ID NO: 50), an anti-TACI moiety (e.g., an anti-TACI scFv such as those disclosed; see Table 2 above, such as SEQ ID NO: 75), one or more intracellular signaling domains such as co-stimulatory signaling domains and cytoplasmic signaling domains, and optionally a hinge domain and a transmembrane domain as disclosed herein. In some instances, the anti-BCMA/anti-TACI bispecific CAR may be a single polypeptide comprising both the anti-BCMA moiety and the anti-TACI moiety. In other instances, the anti-BCMA/anti-TACI bispecific CAR may be a multiple-chain (e.g., 2-chain) molecule. The anti-BCMA moiety and the anti-TACI moiety may be located on separate polypeptides.

In some embodiments, the anti-BCMA/anti-TACI bispecific CAR disclosed herein may comprise an anti-BCMA binding moiety (e.g., scFv) derived from BC-06 and an anti-TACI binding moiety (e.g., scFv) derived from TC-01.

The anti-BCMA binding moiety derived from BC-06 (e.g., scFv) may be any of the anti-BCMA moieties relating to BC-06 disclosed above. In some instances, it may comprise the same heavy chain and/or light chain CDRs as BC-06. In specific examples, the scFv may comprise the same V_{H} and/or same V_{L} as BC-06. In some instances, the scFv may comprise, from the N-terminus to the C-terminus, a V_{L} fragment (e.g., SEQ ID NO: 49), a peptide linker (e.g., SEQ ID NO: 104), and a V_{H} fragment (e.g., SEQ ID NO: 45). Alternatively, the scFv may comprise, from the N-terminus to the C-terminus, a V_{H} fragment (e.g., SEQ ID NO: 45), a peptide linker (e.g., SEQ ID NO: 104), and a V_{L} fragment (e.g., SEQ ID NO: 49). In one specific example, the anti-BCMA binding moiety may comprise SEQ ID NO: 50.

The anti-TACI binding moiety derived from TC-01 (e.g., scFv) may be any of the anti-TACI binding moieties relating to TC-01 disclosed above. In some instances, it comprises the same heavy chain and/or light chain CDRs as TC-01. In specific examples, the scFv may comprise the same V_{H} and/or same V_{L} as TC-01. In some instances, the scFv may comprise, from the N-terminus to the C-terminus, a V_{L} fragment (e.g., SEQ ID NO: 74), a peptide linker (e.g., SEQ ID NO: 104), and a V_{H} fragment (e.g., SEQ ID NO: 70). Alternatively, the scFv may comprise, from the N-terminus to the C-terminus, a V_{H} fragment (e.g., SEQ ID NO: 70), a peptide linker (e.g., SEQ ID NO: 104), and a V_{L} fragment (e.g., SEQ ID NO: 74). In one specific example, the anti-TACI moiety may comprise SEQ ID NO: 75.

In some embodiments, the bi-specific anti-BCMA/anti-TACI binding moieties may be located on a single polypeptide. In some examples, the single polypeptide contains a spacer (a peptide linker) between the anti-BCMA binding moiety and the anti-TACI binding moiety. Examples are provided in Table 3. See also Examples below.

Any of the fusion polypeptide comprising the anti-BCMA and anti-TACI moieties may further comprise a co-stimulatory signaling domain and a cytoplasmic signaling domain such as those disclosed herein. Optionally, the fusion polypeptide may further comprise a hinge domain and a transmembrane domain as also disclosed herein. In some examples, the bispecific CAR can be included in a multi-cistronic expression cassette with an armor gene (e.g., those listed in Table 11 below) via a self-cleavage peptide linker.

### (e) Anti-BCMA or Anti-TACI Monospecific CARs

Also within the scope are anti-BCMA or anti-TACI monospecific CARs comprising any of the anti-BCMA binding moieties or anti-TACI binding moieties as disclosed herein.

In some aspects, provided herein are anti-BCMA CAR, nucleic acids encoding such, and host cells expressing such. The anti-BCMA CAR may comprise (a) an extracellular binding domain which can be any of the anti-BCMA binding moieties, e.g., an anti-BCMA scFv derived from any of the reference antibodies provided in Table 1 above (e.g., BC-05, BC-06, or BC-08); (b) a co-stimulatory signaling domain such as those disclosed herein; and (c) a cytoplasmic signaling domain such as those disclosed herein. The anti-BCMA CAR may further comprise a hinge domain and a transmembrane domain located at the C-terminal of the extracellular antigen binding domain. In one example, the anti-BCMA CAR comprises the amino acid sequence of any one of SEQ ID NO: 119-142 (e.g., SEQ ID NO: 127 or 128).

In some aspects, provided herein are anti-TACI CAR, nucleic acids encoding such, and host cells expressing such. In some examples, the anti-TACI CAR may comprise (a) an extracellular binding domain which can be any of the anti-TACI binding moieties, e.g., an anti-TACI scFv derived from any of the reference antibodies provided in Table 2 above; (b) a co-stimulatory signaling domain such as those disclosed herein; and (c) a cytoplasmic signaling domain such as those disclosed herein. The anti-TACI CAR may further comprise a hinge domain and a transmembrane domain located at the C-terminal of the extracellular antigen binding domain. In one example, the anti-TACI CAR comprises any one of amino acid sequences of SEQ ID NOs: 143-164 (e.g., SEQ ID NO: 143 or 144).

Exemplary anti-BCMA monospecific CARs, anti-TACI monospecific CARs, and anti-BCMA/anti-TACI bispecific CARs are provided in Table 4 below, all of which are within the scope of the present disclosure.

TABLE 4
Exemplary Monospecific and Bispecific CAR Constructs

| | | | SEQ |
|---|---|---|---|
| Exemplary CAR | | Amino Acid Sequence | ID |
| EPLV102/EPLV257 | | MLLLVTSLLLCELPHPAFLLIPEVQLVESGGGLVQPGRSLRLSCAA | 119 |
| (BC-06 scFv), BCMA | | SGFTFDDYAMHWVRQAPGKGLEWVSGISWNSGSIGYADSVKGRFTI | 120 |
| Monospecific CAR, | | SRDNAKNSLYLQMNSLRAEDTALYYCAKVQPPGAFDIWGQGTMVTV | (no signal) |
| medium spacer | | SSGGGGSGGGGSGGGGSDIQLTQSPSFLSASVGDRVSITCRSTQGI | |
| | | SSYLGWYQQKPGKAPKLLIYATSTLQSGVPSRFSGSGSGTEFTLTI | |
| | | SSLQPEDFATYYCQQLYSFGQGTKLEIKGSESKYGPPCPPCPPREP | |
| | | QVYTLPPSQEEMTKNQVSLTCLVKGFYPSDIAVEWESNGQPENNYK | |
| | | TTPPVLDSDGSFFLYSKLTVDKSRWQEGNVFSCSVMHEALHNHYTQ | |
| | | ESLSLSPMFWVLVVVGGVLACYSLLVTVAFIIFWVKRGRKKLLYIF | |
| | | KQPFMRPVQTTQEEDGCSCRFPEEEEGGCELRVKFSRSADAPAYQQ | |
| | | GQNQLYNELNLGRREEYDVLDKRRGRDPEMGGKPRRKNPQEGLYNE | |
| | | LQKDKMAEAYSEIGMKGERRRGKGHDGLYQGLSTATKDTYDALHMQ | |
| | | ALPPR | |
| EPLV102/EPLV257 | | MLLLVTSLLLCELPHPAFLLIPEVQLVESGGGLVQPGRSLRLSCAA | 121 |
| (BC-06 scFv), BCMA | | SGFTFDDYAMHWVRQAPGKGLEWVSGISWNSGSIGYADSVKGRFTI | 122 |
| Monospecific CAR, | | SRDNAKNSLYLQMNSLRAEDTALYYCAKVQPPGAFDIWGQGTMVTV | (no signal) |
| medium spacer (with | | SSGGGGSGGGGSGGGGSDIQLTQSPSFLSASVGDRVSITCRSTQGI | |
| Flag tag) | | SSYLGWYQQKPGKAPKLLIYATSTLQSGVPSRFSGSGSGTEFTLTI | |
| | | SSLQPEDFATYYCQQLYSFGQGTKLEIKGSDYKDDDDKESKYGPPC | |
| | | PPCPPREPQVYTLPPSQEEMTKNQVSLTCLVKGFYPSDIAVEWESN | |
| | | GQPENNYKTTPPVLDSDGSFFLYSKLTVDKSRWQEGNVFSCSVMHE | |
| | | ALHNHYTQESLSLSPMFWVLVVVGGVLACYSLLVTVAFIIFWVKRG | |
| | | RKKLLYIFKQPFMRPVQTTQEEDGCSCRFPEEEEGGCELRVKFSRS | |
| | | ADAPAYQQGQNQLYNELNLGRREEYDVLDKRRGRDPEMGGKPRRKN | |
| | | PQEGLYNELQKDKMAEAYSEIGMKGERRRGKGHDGLYQGLSTATKD | |
| | | TYDALHMQALPPR | |
| EPLV309 BCMA (BC- | | MLLLVTSLLLCELPHPAFLLIPEVQLVESGGGLVQPGRSLRLSCAA | 123 |
| 06) CAR Short Spacer | | SGFTFDDYAMHWVRQAPGKGLEWVSGISWNSGSIGYADSVKGRFTI | 124 |
| | | SRDNAKNSLYLQMNSLRAEDTALYYCAKVQPPGAFDIWGQGTMVTV | (no signal) |
| | | SSGGGGSGGGGSGGGGSDIQLTQSPSFLSASVGDRVSITCRSTQGI | |
| | | SSYLGWYQQKPGKAPKLLIYATSTLQSGVPSRFSGSGSGTEFTLTI | |
| | | SSLQPEDFATYYCQQLYSFGQGTKLEIKGSESKYGPPCPPCPMFWV | |
| | | LVVVGGVLACYSLLVTVAFIIFWVKRGRKKLLYIFKQPFMRPVQTT | |
| | | QEEDGCSCRFPEEEEGGCELRVKFSRSADAPAYQQGQNQLYNELNL | |
| | | GRREEYDVLDKRRGRDPEMGGKPRRKNPQEGLYNELQKDKMAEAYS | |
| | | EIGMKGERRRGKGHDGLYQGLSTATKDTYDALHMQALPPR | |
| EPLV309 BCMA (BC- | | MLLLVTSLLLCELPHPAFLLIPEVQLVESGGGLVQPGRSLRLSCAA | 125 |
| 06) CAR Short Spacer | | SGFTFDDYAMHWVRQAPGKGLEWVSGISWNSGSIGYADSVKGRFTI | 126 |
| (with Flag tag) | | SRDNAKNSLYLQMNSLRAEDTALYYCAKVQPPGAFDIWGQGTMVTV | (no signal) |
| | | SSGGGGSGGGGSGGGGSDIQLTQSPSFLSASVGDRVSITCRSTQGI | |
| | | SSYLGWYQQKPGKAPKLLIYATSTLQSGVPSRFSGSGSGTEFTLTI | |
| | | SSLQPEDFATYYCQQLYSFGQGTKLEIKGSDYKDDDDKESKYGPPC | |
| | | PPCPMFWVLVVVGGVLACYSLLVTVAFIIFWVKRGRKKLLYIFKQP | |
| | | FMRPVQTTQEEDGCSCRFPEEEEGGCELRVKFSRSADAPAYQQGQN | |
| | | QLYNELNLGRREEYDVLDKRRGRDPEMGGKPRRKNPQEGLYNELQK | |
| | | DKMAEAYSEIGMKGERRRGKGHDGLYQGLSTATKDTYDALHMQALP | |
| | | PR | |
| EPLV310 BCMA (BC- | | MLLLVTSLLLCELPHPAFLLIPEVQLVESGGGLVQPGRSLRLSCAA | 127 |
| 06) CAR Medium | | SGFTFDDYAMHWVRQAPGKGLEWVSGISWNSGSIGYADSVKGRFTI | 128 |
| Spacer (Spacer 1) | | SRDNAKNSLYLQMNSLRAEDTALYYCAKVQPPGAFDIWGQGTMVTV | (no signal) |
| | | SSGGGGSGGGGSGGGGSDIQLTQSPSFLSASVGDRVSITCRSTQGI | |
| | | SSYLGWYQQKPGKAPKLLIYATSTLQSGVPSRFSGSGSGTEFTLTI | |
| | | SSLQPEDFATYYCQQLYSFGQGTKLEIKGSESKYGPPCPPCPGQPR | |
| | | EPQVYTLPPSQEEMTKNQVSLTCLVKGFYPSDIAVEWESNGQPENN | |
| | | YKTTPPVLDSDGSFFLYSKLTVDKSRWQEGNVFSCSVMHEALHNHY | |
| | | TQESLSLSPMFWVLVVVGGVLACYSLLVTVAFIIFWVKRGRKKLLY | |
| | | IFKQPFMRPVQTTQEEDGCSCRFPEEEEGGCELRVKFSRSADAPAY | |
| | | QQGQNQLYNELNLGRREEYDVLDKRRGRDPEMGGKPRRKNPQEGLY | |
| | | NELQKDKMAEAYSEIGMKGERRRGKGHDGLYQGLSTATKDTYDALH | |
| | | MQALPPR | |
| EPLV310 BCMA (BC- | | MLLLVTSLLLCELPHPAFLLIPEVQLVESGGGLVQPGRSLRLSCAA | 129 |
| 06) CAR Medium | | SGFTFDDYAMHWVRQAPGKGLEWVSGISWNSGSIGYADSVKGRFTI | 130 |
| Spacer (Spacer 1) (with | | SRDNAKNSLYLQMNSLRAEDTALYYCAKVQPPGAFDIWGQGTMVTV | (no signal) |
| Flag tag) | | SSGGGGSGGGGSGGGGSDIQLTQSPSFLSASVGDRVSITCRSTQGI | |
| | | SSYLGWYQQKPGKAPKLLIYATSTLQSGVPSRFSGSGSGTEFTLTI | |
| | | SSLQPEDFATYYCQQLYSFGQGTKLEIKGSDYKDDDDKESKYGPPC | |
| | | PPCPGQPREPQVYTLPPSQEEMTKNQVSLTCLVKGFYPSDIAVEWE | |
| | | SNGQPENNYKTTPPVLDSDGSFFLYSKLTVDKSRWQEGNVFSCSVM | |
| | | HEALHNHYTQESLSLSPMFWVLVVVGGVLACYSLLVTVAFIIFWVK | |
| | | RGRKKLLYIFKQPFMRPVQTTQEEDGCSCRFPEEEEGGCELRVKFS | |
| | | RSADAPAYQQGQNQLYNELNLGRREEYDVLDKRRGRDPEMGGKPRR | |
| | | KNPQEGLYNELQKDKMAEAYSEIGMKGERRRGKGHDGLYQGLSTAT | |
| | | KDTYDALHMQALPPR | |
| EPLV311 BCMA (BC- | | MLLLVTSLLLCELPHPAFLLIPEVQLVESGGGLVQPGRSLRLSCAA | 131 |
| 06) CAR Long Spacer | | SGFTFDDYAMHWVRQAPGKGLEWVSGISWNSGSIGYADSVKGRFTI | 132 |
| (Spacer 2) | | SRDNAKNSLYLQMNSLRAEDTALYYCAKVQPPGAFDIWGQGTMVTV | (no signal) |
| | | SSGGGGSGGGGSGGGGSDIQLTQSPSFLSASVGDRVSITCRSTQGI | |
| | | SSYLGWYQQKPGKAPKLLIYATSTLQSGVPSRFSGSGSGTEFTLTI | |
| | | SSLQPEDFATYYCQQLYSFGQGTKLEIKGSESKYGPPCPPCPAPEF | |
| | | EGAPSVFLFPPKPKDTLMISRTPEVTCVVVDVSQEDPEVQFNWYVD | |
| | | GVEVHNAKTKPREEQFQSTYRVVSVLTVLHQDWLNGKEYKCKVSNK | |
| | | GLPSSIEKTISKAKGQPREPQVYTLPPSQEEMTKNQVSLTCLVKGF | |
| | | YPSDIAVEWESNGQPENNYKTTPPVLDSDGSFFLYSKLTVDKSRWQ | |
| | | EGNVFSCSVMHEALHNHYTQESLSLSPMFWVLVVVGGVLACYSLLV | |
| | | TVAFIIFWVKRGRKKLLYIFKQPFMRPVQTTQEEDGCSCRFPEEEE | |
| | | GGCELRVKFSRSADAPAYQQGQNQLYNELNLGRREEYDVLDKRRGR | |
| | | DPEMGGKPRRKNPQEGLYNELQKDKMAEAYSEIGMKGERRRGKGHD | |
| | | GLYQGLSTATKDTYDALHMQALPPR | |
| EPLV311 BCMA (BC- | | MLLLVTSLLLCELPHPAFLLIPEVQLVESGGGLVQPGRSLRLSCAA | 133 |
| 06) CAR Long Spacer | | SGFTFDDYAMHWVRQAPGKGLEWVSGISWNSGSIGYADSVKGRFTI | 134 |
| (Spacer 2) (with Flag | | SRDNAKNSLYLQMNSLRAEDTALYYCAKVQPPGAFDIWGQGTMVTV | (no signal) |
| tag) | | SSGGGGSGGGGSGGGGSDIQLTQSPSFLSASVGDRVSITCRSTQGI | |
| | | SSYLGWYQQKPGKAPKLLIYATSTLQSGVPSRFSGSGSGTEFTLTI | |
| | | SSLQPEDFATYYCQQLYSFGQGTKLEIKGSDYKDDDDKESKYGPPC | |
| | | PPCPAPEFEGAPSVFLFPPKPKDTLMISRTPEVTCVVVDVSQEDPE | |
| | | VQFNWYVDGVEVHNAKTKPREEQFQSTYRVVSVLTVLHQDWLNGKE | |
| | | YKCKVSNKGLPSSIEKTISKAKGQPREPQVYTLPPSQEEMTKNQVS | |
| | | LTCLVKGFYPSDIAVEWESNGQPENNYKTTPPVLDSDGSFFLYSKL | |
| | | TVDKSRWQEGNVFSCSVMHEALHNHYTQESLSLSPMFWVLVVVGGV | |
| | | LACYSLLVTVAFIIFWVKRGRKKLLYIFKQPFMRPVQTTQEEDGCS | |
| | | CRFPEEEEGGCELRVKFSRSADAPAYQQGQNQLYNELNLGRREEYD | |
| | | VLDKRRGRDPEMGGKPRRKNPQEGLYNELQKDKMAEAYSEIGMKGE | |
| | | RRRGKGHDGLYQGLSTATKDTYDALHMQALPPR | |
| EPLV103 (BC-08 scFv), | | MLLLVTSLLLCELPHPAFLLIPQVQLVQSGGGVVQPGRSLRLSCAA | 135 |
| BCMA monospecific | | SGFTFSSYAMHWVRQAPGKGLEWVAVISYDGSNKYYADSVKGRFTI | 136 |
| CAR, medium spacer | | SRDNAENSVYLQMNSLRAEDTAVYYCARDEHQVVPNYRFDFWGQGS | (no signal) |
| (Spacer 1) | | QVTVSSGGGGSGGGGSGGGGSQLVLTQPPSASGTPGQRVTISCSGS | |
| | | GSNIGSNDVSWYQQIPGTASKLLIYWNDQRPSGVPDRFSGSKSGTS | |
| | | ASLAISGLQSEDEADYYCAAWDDSLNGWVFGGGTKLTVLGSESKYG | |
| | | PPCPPCPGQPREPQVYTLPPSQEEMTKNQVSLTCLVKGFYPSDIAV | |
| | | EWESNGQPENNYKTTPPVLDSDGSFFLYSKLTVDKSRWQEGNVFSC | |
| | | SVMHEALHNHYTQESLSLSPMFWVLVVVGGVLACYSLLVTVAFIIF | |
| | | WVKRGRKKLLYIFKQPFMRPVQTTQEEDGCSCRFPEEEEGGCELRV | |
| | | KFSRSADAPAYQQGQNQLYNELNLGRREEYDVLDKRRGRDPEMGGK | |
| | | PRRKNPQEGLYNELQKDKMAEAYSEIGMKGERRRGKGHDGLYQGLS | |
| | | TATKDTYDALHMQALPPR | |
| EPLV103 (BC-08 scFv), | | MLLLVTSLLLCELPHPAFLLIPQVQLVQSGGGVVQPGRSLRLSCAA | 137 |
| BCMA monospecific | | SGFTFSSYAMHWVRQAPGKGLEWVAVISYDGSNKYYADSVKGRFTI | 138 |
| CAR, medium spacer | | SRDNAENSVYLQMNSLRAEDTAVYYCARDEHQVVPNYRFDFWGQGS | (no signal) |
| (Spacer 1) (with Flag | | QVTVSSGGGGSGGGGSGGGGSQLVLTQPPSASGTPGQRVTISCSGS | |
| tag) | | GSNIGSNDVSWYQQIPGTASKLLIYWNDQRPSGVPDRFSGSKSGTS | |
| | | ASLAISGLQSEDEADYYCAAWDDSLNGWVFGGGTKLTVLGSDYKDD | |
| | | DDKESKYGPPCPPCPGQPREPQVYTLPPSQEEMTKNQVSLTCLVKG | |
| | | FYPSDIAVEWESNGQPENNYKTTPPVLDSDGSFFLYSKLTVDKSRW | |
| | | QEGNVFSCSVMHEALHNHYTQESLSLSPMFWVLVVVGGVLACYSLL | |
| | | VTVAFIIFWVKRGRKKLLYIFKQPFMRPVQTTQEEDGCSCRFPEEE | |
| | | EGGCELRVKFSRSADAPAYQQGQNQLYNELNLGRREEYDVLDKRRG | |
| | | RDPEMGGKPRRKNPQEGLYNELQKDKMAEAYSEIGMKGERRRGKGH | |
| | | DGLYQGLSTATKDTYDALHMQALPPR | |
| EPLV104 (BC-05 scFv), | | MLLLVTSLLLCELPHPAFLLIPQVQLVQSGGGVVQPGGSLRLSCAA | 139 |
| BCMA monospecific | | SGFTFSSYGMHWVRQAPGKGLEWVAFIRYDGSNKYYADSVKGRFTI | 140 |
| CAR, medium spacer | | SRDNSKNTLYLQMNSLRAEDTAVYYCAKDWEDPLYYYDTPFWGQGT | (no signal) |
| (Spacer 1) | | LVTVSSGGGGSGGGGSGGGGSVIWMTQSPSSVSASVGDIITITCQA | |
| | | SQDINNYLNWYQLKPGKAPKLLIYDASNVETGVPSRFSGSGSGTDF | |
| | | TFTISSLQPEDTATYYCQQYDKLPYTFGQGTKLEIKGSESKYGPPC | |
| | | PPCPGQPREPQVYTLPPSQEEMTKNQVSLTCLVKGFYPSDIAVEWE | |
| | | SNGQPENNYKTTPPVLDSDGSFFLYSKLTVDKSRWQEGNVFSCSVM | |
| | | HEALHNHYTQESLSLSPMFWVLVVVGGVLACYSLLVTVAFIIFWVK | |
| | | RGRKKLLYIFKQPFMRPVQTTQEEDGCSCRFPEEEEGGCELRVKFS | |
| | | RSADAPAYQQGQNQLYNELNLGRREEYDVLDKRRGRDPEMGGKPRR | |
| | | KNPQEGLYNELQKDKMAEAYSEIGMKGERRRGKGHDGLYQGLSTAT | |
| | | KDTYDALHMQALPPR | |
| EPLV104 (BC-05 scFv), | | MLLLVTSLLLCELPHPAFLLIPQVQLVQSGGGVVQPGGSLRLSCAA | 141 |
| BCMA monospecific | | SGFTFSSYGMHWVRQAPGKGLEWVAFIRYDGSNKYYADSVKGRFTI | 142 |
| CAR, medium spacer | | SRDNSKNTLYLQMNSLRAEDTAVYYCAKDWEDPLYYYDTPFWGQGT | (no signal) |
| (Spacer 1) (with Flag | | LVTVSSGGGGSGGGGSGGGGSVIWMTQSPSSVSASVGDIITITCQA | |
| tag) | | SQDINNYLNWYQLKPGKAPKLLIYDASNVETGVPSRFSGSGSGTDF | |
| | | TFTISSLQPEDTATYYCQQYDKLPYTFGQGTKLEIKGSDYKDDDDK | |
| | | ESKYGPPCPPCPGQPREPQVYTLPPSQEEMTKNQVSLTCLVKGFYP | |
| | | SDIAVEWESNGQPENNYKTTPPVLDSDGSFFLYSKLTVDKSRWQEG | |
| | | NVFSCSVMHEALHNHYTQESLSLSPMFWVLVVVGGVLACYSLLVTV | |
| | | AFIIFWVKRGRKKLLYIFKQPFMRPVQTTQEEDGCSCRFPEEEEGG | |
| | | CELRVKFSRSADAPAYQQGQNQLYNELNLGRREEYDVLDKRRGRDP | |
| | | EMGGKPRRKNPQEGLYNELQKDKMAEAYSEIGMKGERRRGKGHDGL | |
| | | YQGLSTATKDTYDALHMQALPPR | |
| EPLV300, TACI (TC-01 | | MLLLVTSLLLCELPHPAFLLIPQVQLVQSGAEVKKPGASVKVSCKA | 143 |
| scFv) mono-specific | | SGYTFTGYFMHWVRQAPGQGLEWMGWINSNSGGTNYAQKFQGRVTM | 144 |
| CAR (short spacer) | | TRDTSISTAYMELSRLRSDDTAVYYCARDDGTGYNAFDIWGQGTMV | (no signal) |
| | | TVSSGGGGSGGGGSGGGGSDIVMTQSPLSLPVTPGEPASISCRSSQ | |
| | | SLLHSNGYNYLDWYVQKPGKPPQLLIYLASHRASGVPDRFSGSGSG | |
| | | TDFTLKISRVEAEDVGIYYCMQAQQTALFGPGTKVDIKGSESKYGP | |
| | | PCPPCPMFWVLVVVGGVLACYSLLVTVAFIIFWVKRGRKKLLYIFK | |
| | | QPFMRPVQTTQEEDGCSCRFPEEEEGGCELRVKFSRSADAPAYQQG | |
| | | QNQLYNELNLGRREEYDVLDKRRGRDPEMGGKPRRKNPQEGLYNEL | |
| | | QKDKMAEAYSEIGMKGERRRGKGHDGLYQGLSTATKDTYDALHMQA | |
| | | LPPR | |
| EPLV300, TACI (TC-01 | | MLLLVTSLLLCELPHPAFLLIPQVQLVQSGAEVKKPGASVKVSCKA | 145 |
| scFv) mono-specific | | SGYTFTGYFMHWVRQAPGQGLEWMGWINSNSGGTNYAQKFQGRVTM | 146 |
| CAR (short spacer) | | TRDTSISTAYMELSRLRSDDTAVYYCARDDGTGYNAFDIWGQGTMV | (no signal) |
| (with Flag tag) | | TVSSGGGGSGGGGSGGGGSDIVMTQSPLSLPVTPGEPASISCRSSQ | |
| | | SLLHSNGYNYLDWYVQKPGKPPQLLIYLASHRASGVPDRFSGSGSG | |
| | | TDFTLKISRVEAEDVGIYYCMQAQQTALFGPGTKVDIKGSDYKDDD | |
| | | DKESKYGPPCPPCPMFWVLVVVGGVLACYSLLVTVAFIIFWVKRGR | |
| | | KKLLYIFKQPFMRPVQTTQEEDGCSCRFPEEEEGGCELRVKFSRSA | |
| | | DAPAYQQGQNQLYNELNLGRREEYDVLDKRRGRDPEMGGKPRRKNP | |
| | | QEGLYNELQKDKMAEAYSEIGMKGERRRGKGHDGLYQGLSTATKDT | |
| | | YDALHMQALPPR | |
| EPLV301, TACI (TC-01 | | MLLLVTSLLLCELPHPAFLLIPQVQLVQSGAEVKKPGASVKVSCKA | 147 |
| scFv) mono-specific | | SGYTFTGYFMHWVRQAPGQGLEWMGWINSNSGGTNYAQKFQGRVTM | 148 |
| CAR (Medium spacer; | | TRDTSISTAYMELSRLRSDDTAVYYCARDDGTGYNAFDIWGQGTMV | (no signal) |
| Spacer 1) | | TVSSGGGGSGGGGSGGGGSDIVMTQSPLSLPVTPGEPASISCRSSQ | |
| | | SLLHSNGYNYLDWYVQKPGKPPQLLIYLASHRASGVPDRFSGSGSG | |
| | | TDFTLKISRVEAEDVGIYYCMQAQQTALFGPGTKVDIKGSESKYGP | |
| | | PCPPCPGQPREPQVYTLPPSQEEMTKNQVSLTCLVKGFYPSDIAVE | |
| | | WESNGQPENNYKTTPPVLDSDGSFFLYSKLTVDKSRWQEGNVFSCS | |
| | | VMHEALHNHYTQESLSLSPMFWVLVVVGGVLACYSLLVTVAFIIFW | |
| | | VKRGRKKLLYIFKQPFMRPVQTTQEEDGCSCRFPEEEEGGCELRVK | |
| | | FSRSADAPAYQQGQNQLYNELNLGRREEYDVLDKRRGRDPEMGGKP | |
| | | RRKNPQEGLYNELQKDKMAEAYSEIGMKGERRRGKGHDGLYQGLST | |
| | | ATKDTYDALHMQALPPR | |
| EPLV301, TACI (TC-01 | | MLLLVTSLLLCELPHPAFLLIPQVQLVQSGAEVKKPGASVKVSCKA | 149 |
| scFv) mono-specific | | SGYTFTGYFMHWVRQAPGQGLEWMGWINSNSGGTNYAQKFQGRVTM | 150 |
| CAR (Medium spacer; | | TRDTSISTAYMELSRLRSDDTAVYYCARDDGTGYNAFDIWGQGTMV | (no signal) |
| Spacer 1) (with Flag | | TVSSGGGGSGGGGSGGGGSDIVMTQSPLSLPVTPGEPASISCRSSQ | |
| tag) | | SLLHSNGYNYLDWYVQKPGKPPQLLIYLASHRASGVPDRFSGSGSG | |
| | | TDFTLKISRVEAEDVGIYYCMQAQQTALFGPGTKVDIKGSDYKDDD | |
| | | DKESKYGPPCPPCPGQPREPQVYTLPPSQEEMTKNQVSLTCLVKGF | |
| | | YPSDIAVEWESNGQPENNYKTTPPVLDSDGSFFLYSKLIVDKSRWQ | |
| | | EGNVFSCSVMHEALHNHYTQESLSLSPMFWVLVVVGGVLACYSLLV | |
| | | TVAFIIFWVKRGRKKLLYIFKQPFMRPVQTTQEEDGCSCRFPEEEE | |
| | | GGCELRVKFSRSADAPAYQQGQNQLYNELNLGRREEYDVLDKRRGR | |
| | | DPEMGGKPRRKNPQEGLYNELQKDKMAEAYSEIGMKGERRRGKGHD | |
| | | GLYQGLSTATKDTYDALHMQALPPR | |
| EPLV319, TACI (TC-03 | | MLLLVTSLLLCELPHPAFLLIPEVQLVESGGGLVQPGGSLRLSCAT | 151 |
| scFv) mono-specific | | SGFTVNSDYMSWVRQSPEKGLEWVSFIYRGGSTYYADSVKGRFTVS | 152 |
| CAR (Short spacer) | | RDTSKNTLYLQMNSLRVEDTAVYYCARGLGSSSDYWGQGTLVTVSS | (no signal) |
| | | GGGGSGGGGSGGGGSSYELTQPHSVSESPGKTVTISCTRSSGSIAN | |
| | | NYVQWYRHRPGSAPSTVIFEDDHRPSEVPDRFSGSIDSSSNSASLT | |
| | | ISGLKTEDEADYYCQSYDGIDHYVFGTGTKLTVLGSESKYGPPCPP | |
| | | CPMFWVLVVVGGVLACYSLLVTVAFIIFWVKRGRKKLLYIFKQPFM | |
| | | RPVQTTQEEDGCSCRFPEEEEGGCELRVKFSRSADAPAYQQGQNQL | |
| | | YNELNLGRREEYDVLDKRRGRDPEMGGKPRRKNPQEGLYNELQKDK | |
| | | MAEAYSEIGMKGERRRGKGHDGLYQGLSTATKDTYDALHMQALPPR | |
| EPLV319, TACI (TC-03 | | MLLLVTSLLLCELPHPAFLLIPEVQLVESGGGLVQPGGSLRLSCAT | 153 |
| scFv) mono-specific | | SGFTVNSDYMSWVRQSPEKGLEWVSFIYRGGSTYYADSVKGRFTVS | 154 |
| CAR (Short spacer) | | RDTSKNTLYLQMNSLRVEDTAVYYCARGLGSSSDYWGQGTLVTVSS | (no signal) |
| (with Flag tag) | | GGGGSGGGGSGGGGSSYELTQPHSVSESPGKTVTISCTRSSGSIAN | |
| | | NYVQWYRHRPGSAPSTVIFEDDHRPSEVPDRFSGSIDSSSNSASLT | |
| | | ISGLKTEDEADYYCQSYDGIDHYVFGTGTKLTVLGSDYKDDDDKES | |
| | | KYGPPCPPCPMFWVLVVVGGVLACYSLLVTVAFIIFWVKRGRKKLL | |
| | | YIFKQPFMRPVQTTQEEDGCSCRFPEEEEGGCELRVKFSRSADAPA | |
| | | YQQGQNQLYNELNLGRREEYDVLDKRRGRDPEMGGKPRRKNPQEGL | |
| | | YNELQKDKMAEAYSEIGMKGERRRGKGHDGLYQGLSTATKDTYDAL | |
| | | HMQALPPR | |
| EPLV320, TACI (TC-04 | | MLLLVTSLLLCELPHPAFLLIPEVQLVQSGAEVKKPGSSVKVSCKA | 155 |
| scFv) mono-specific | | SGGTFSSYAISWVRQAPGQGLEWMGGIIPIFGTANYAQKFQGRVTI | 156 |
| CAR (Short spacer) | | TADKSTSTAYMELSSLRSEDTAVYYCARDKANTALRWFDPWGQGTL | (no signal) |
| | | VTVSSGGGGSGGGGSGGGGSEIVMTQSPGSLSLSPGDTATLSCRAS | |
| | | QRVSSSYLAWYQQKPGQAPRLLIYGASKRATGISSKFSGSGSETDF | |
| | | LLTISRLEPEDIAVYYCQQYGDSPLTFGQGTKVEIKGSESKYGPPC | |
| | | PPCPMFWVLVVVGGVLACYSLLVTVAFIIFWVKRGRKKLLYIFKQP | |
| | | FMRPVQTTQEEDGCSCRFPEEEEGGCELRVKFSRSADAPAYQQGQN | |
| | | QLYNELNLGRREEYDVLDKRRGRDPEMGGKPRRKNPQEGLYNELQK | |
| | | DKMAEAYSEIGMKGERRRGKGHDGLYQGLSTATKDTYDALHMQALP | |
| | | PR | |
| EPLV320, TACI (TC-04 | | MLLLVTSLLLCELPHPAFLLIPEVQLVQSGAEVKKPGSSVKVSCKA | 157 |
| scFv) mono-specific | | SGGTFSSYAISWVRQAPGQGLEWMGGIIPIFGTANYAQKFQGRVTI | 158 |
| CAR (Short spacer) | | TADKSTSTAYMELSSLRSEDTAVYYCARDKANTALRWFDPWGQGTL | (no signal) |
| (with Flag tag) | | VTVSSGGGGSGGGGSGGGGSEIVMTQSPGSLSLSPGDTATLSCRAS | |
| | | QRVSSSYLAWYQQKPGQAPRLLIYGASKRATGISSKFSGSGSETDF | |
| | | LLTISRLEPEDIAVYYCQQYGDSPLTFGQGTKVEIKGSDYKDDDDK | |
| | | ESKYGPPCPPCPMFWVLVVVGGVLACYSLLVTVAFIIFWVKRGRKK | |
| | | LLYIFKQPFMRPVQTTQEEDGCSCRFPEEEEGGCELRVKFSRSADA | |
| | | PAYQQGQNQLYNELNLGRREEYDVLDKRRGRDPEMGGKPRRKNPQE | |
| | | GLYNELQKDKMAEAYSEIGMKGERRRGKGHDGLYQGLSTATKDTYD | |
| | | ALHMQALPPR | |
| EPLV321, TACI (TC-02 | | MLLLVTSLLLCELPHPAFLLIPQVQLQQWGAGLLKPSETLSLTCAV | 159 |
| scFV) mono-specific | | YGGSFSGYYWSWIRQPPGKGLEWIGEINHSGSTNYNPSLKSRVTIS | 160 |
| CAR (Short spacer) | | VDTSKNQFSLKLSSVTAADTAVYYCARIRKLGGYYFDYWGQGTLVT | (no signal) |
| | | VSSGGGGSGGGGSGGGGSEIVMTQSPGTLSLSPGERATLSCRASQS | |
| | | VSSSYLAWYQQKPGQAPRLLIYGASSRATGIPDRFSGSGSGTDFTL | |
| | | TISRLEPEDFAVYYCQQYGSSPRPFGQGTKVEIKGSESKYGPPCPP | |
| | | CPMFWVLVVVGGVLACYSLLVTVAFIIFWVKRGRKKLLYIFKQPFM | |
| | | RPVQTTQEEDGCSCRFPEEEEGGCELRVKFSRSADAPAYQQGQNQL | |
| | | YNELNLGRREEYDVLDKRRGRDPEMGGKPRRKNPQEGLYNELQKDK | |
| | | MAEAYSEIGMKGERRRGKGHDGLYQGLSTATKDTYDALHMQALPPR | |
| EPLV321, TACI (TC-02 | | MLLLVTSLLLCELPHPAFLLIPQVQLQQWGAGLLKPSETLSLTCAV | 161 |
| scFV) mono-specific | | YGGSFSGYYWSWIRQPPGKGLEWIGEINHSGSTNYNPSLKSRVTIS | 162 |
| CAR (Short spacer) | | VDTSKNQFSLKLSSVTAADTAVYYCARIRKLGGYYFDYWGQGTLVT | (no signal) |
| (with Flag tag) | | VSSGGGGSGGGGSGGGGSEIVMTQSPGTLSLSPGERATLSCRASQS | |
| | | VSSSYLAWYQQKPGQAPRLLIYGASSRATGIPDRFSGSGSGTDFTL | |
| | | TISRLEPEDFAVYYCQQYGSSPRPFGQGTKVEIKGSDYKDDDDKES | |
| | | KYGPPCPPCPMFWVLVVVGGVLACYSLLVTVAFIIFWVKRGRKKLL | |
| | | YIFKQPFMRPVQTTQEEDGCSCRFPEEEEGGCELRVKFSRSADAPA | |
| | | YQQGQNQLYNELNLGRREEYDVLDKRRGRDPEMGGKPRRKNPQEGL | |
| | | YNELQKDKMAEAYSEIGMKGERRRGKGHDGLYQGLSTATKDTYDAL | |
| | | HMQALPPR | |
| EPLV302, Bispecific | | MLLLVTSLLLCELPHPAFLLIPEVQLVESGGGLVQPGRSLRLSCAA | 163 |
| BCMA (BC-06) and | | SGFTFDDYAMHWVRQAPGKGLEWVSGISWNSGSIGYADSVKGRFTI | 164 |
| TACI (TC-01)- | | SRDNAKNSLYLQMNSLRAEDTALYYCAKVQPPGAFDIWGQGTMVTV | (no signal) |
| Extracellular domain | | SSGGGGSGGGGSGGGGSDIQLTQSPSFLSASVGDRVSITCRSTQGI | |
| | | SSYLGWYQQKPGKAPKLLIYATSTLQSGVPSRFSGSGSGTEFTLTI | |
| | | SSLQPEDFATYYCQQLYSFGQGTKLEIKGGGGSQVQLVQSGAEVKK | |
| | | PGASVKVSCKASGYTFTGYFMHWVRQAPGQGLEWMGWINSNSGGTN | |
| | | YAQKFQGRVTMTRDTSISTAYMELSRLRSDDTAVYYCARDDGTGYN | |
| | | AFDIWGQGTMVTVSSGGGGSGGGGSGGGGSDIVMTQSPLSLPVTPG | |
| | | EPASISCRSSQSLLHSNGYNYLDWYVQKPGKPPQLLIYLASHRASG | |
| | | VPDRFSGSGSGTDFTLKISRVEAEDVGIYYCMQAQQTALFGPGTKV | |
| | | DIK | |
| EPLV302, Bispecific | | MLLLVTSLLLCELPHPAFLLIPEVQLVESGGGLVQPGRSLRLSCAA | 165 |
| BCMA (BC-06) and | | SGFTFDDYAMHWVRQAPGKGLEWVSGISWNSGSIGYADSVKGRFTI | 166 |
| TACI (TC-01) CAR, | | SRDNAKNSLYLQMNSLRAEDTALYYCAKVQPPGAFDIWGQGTMVTV | (no signal) |
| (short spacer) | | SSGGGGSGGGGSGGGGSDIQLTQSPSFLSASVGDRVSITCRSTQGI | |
| | | SSYLGWYQQKPGKAPKLLIYATSTLQSGVPSRFSGSGSGTEFTLTI | |
| | | SSLQPEDFATYYCQQLYSFGQGTKLEIKGGGGSQVQLVQSGAEVKK | |
| | | PGASVKVSCKASGYTFTGYFMHWVRQAPGQGLEWMGWINSNSGGTN | |
| | | YAQKFQGRVTMTRDTSISTAYMELSRLRSDDTAVYYCARDDGTGYN | |
| | | AFDIWGQGTMVTVSSGGGGSGGGGSGGGGSDIVMTQSPLSLPVTPG | |
| | | EPASISCRSSQSLLHSNGYNYLDWYVQKPGKPPQLLIYLASHRASG | |
| | | VPDRFSGSGSGTDFTLKISRVEAEDVGIYYCMQAQQTALFGPGTKV | |
| | | DIKESKYGPPCPPCPMFWVLVVVGGVLACYSLLVTVAFIIFWVKRG | |
| | | RKKLLYIFKQPFMRPVQTTQEEDGCSCRFPEEEEGGCELRVKFSRS | |
| | | ADAPAYQQGQNQLYNELNLGRREEYDVLDKRRGRDPEMGGKPRRKN | |
| | | PQEGLYNELQKDKMAEAYSEIGMKGERRRGKGHDGLYQGLSTATKD | |
| | | TYDALHMQALPPR | |
| EP322 | Bispecific | EPLV302 (see above) | 165 |
| Armored | CAR | | 166 |
| | | | (no signal) |
| | Anti-PDL1- | See Table 11 | 167 |
| | IL2mut-2 | | 168 |
| | Armor | | (no signal) |
| EP323 | Bispecific | EPLV302 (see above) | 165 |
| Armored | CAR | | 166 |
| | | | (no signal) |
| | Membrane- | See Table 11 | 169 |
| | bound | | 170 |
| | IL2mut | | (no signal) |
| | Armor | | |
| EP324 | Bispecific | EPLV302 (see above) | 165 |
| Armored | CAR | | 166 |
| | | | (no signal) |
| | mIL15 | See Table 11 | 171 |
| | Armor | | 172 |
| | | | (no signal) |
| EPLV327 | Bispecific | EPLV302 (see above) | 165 |
| Armored | CAR | | 166 |
| | | | (no signal) |
| | Anti-PDL1- | See Table 11 | 173 |
| | IL2mut | | 174 |
| | Armor | | (no signal) |
| EPLV328 | Bispecific | EPLV302 (see above) | 165 |
| Armored | CAR | | 166 |
| | | | (no signal) |
| | sIL15 Armor | See Table 11 | 175 |
| | | | 176 |
| | | | (no signal) |
| EPLV342 | Bispecific | EPLV302 (see above) | 165 |
| | CAR | | 166 |
| | | | (no signal) |
| | IL2mut | See Table 11 | 177 |
| | Armor | | 178 |
| | | | (no signal) |

### II. CAR-Expressing Immune Cells

In some aspects, provided herein are genetically engineered immune cells such as T cells NK cells, or macrophages having surface expression of any of the anti-BCMA, anti-TACI, or anti-BCMA/TACI bispecific CAR constructs disclosed herein. In some instances, the genetically engineered immune cells are T cells expressing any of the anti-BCMA/TACI bispecific CAR provided in Table 4 above (e.g., SEQ ID NO: 165 or 166).

### (a) Armored CAR-T Cells

Any of the CAR-expression immune cells disclosed herein may be engineered to further with additional mechanisms to reprogram the CAR-expressing cells so as to enhance their bioactivity and/or persistence, thereby enhancing overall therapeutic effects. For example, the CAR-expressing immune cells may be further engineered to express an armor polypeptide to enhance physical and/or biological features of the CAR-T cells. Such CAR-T cells are know as armored CAR-T cells, which co-express one or more CAR constructs and an armor polypeptide that is capable of enhancing CAR-T cell features, e.g., improving growth and/or persistence, enhancing efficacy, reducing toxicities, etc., or a combination thereof.

Exemplary armor polypeptides include, but are not limited to, a suitable cytokine such as IL-2, IL-5, and/or IL-15, a co-stimulatory ligand (e.g., CD80, or CD86), a checkpoint inhibitor (e.g., an anti-PD1 or anti-PDL1 antibody fragment), a soluble receptor such as a soluble PD1, TGFR2 trap, or VEGFR2 trap, and/or an immune cell activation ligand (e.g., 4-1BBL). In some embodiments, the armor polypeptide may be a fusion polypeptide comprising, e.g., a cytokine or a fragment thereof (e.g., IL2 or IL15 or a fragment thereof), and a checkpoint inhibitor (e.g., an anti-PDL1 fragment). Specific examples of armor polypeptides are provided in Table 11 below, each of which is within the scope of the present disclosure.

TABLE 11
Exemplary Armor Polypeptides

| Exemplary Armor | | SEQ |
|---|---|---|
| Polypeptide | Amino Acid Sequence | ID |
| Anti-PDL1- | MALPVTALLLPLALLLHAARPQVQLVQSGAEVKKPGSSVKVSCKAS | 167 |
| IL2mut-2 | GGTFSSYAISWVRQAPGQGLEWMGGIIPIFGTANYAQKFQGRVTIT | 168 (no |
| | ADESTSTAYMELSSLRSEDTAVYYCARDKWYGSAWRGDYWGQGTLV | signal) |
| | TVSSGGGGSGGGGSGGGGSQSVLTQPPSASGTPGQRVTISCSGSYS | |
| | NIGSNFVNWYQQLPGTAPKLLIHFNNQRPSGVPDRFSGSRSGTSAS | |
| | LAISGLQSEDEADYFCSTWDDSLKGPIFGGGTKLTVLGGGGSGGGG | |
| | SGGGGSAPASSSTKKTQLQLEHLLLDLQMILNGINNYKNPLLTDML | |
| | TRKFYMPKKATELKHLQCLEEELKPLEEVLNLAQSKNFHLRPRDLI | |
| | SNISNIVLELKGSETTFMCEYADETATIVEFLNRWITFSQSIISTL | |
| | T | |
| Membrane-bound | MALPVTALLLPLALLLHAARPAPASSSTKKTQLQLEHLLLDLQMIL | 169 |
| IL2mut | NGINNYKNPLLTDMLTRKFYMPKKATELKHLQCLEEELKPLEEVLN | 170 (no |
| | LAQSKNFHLSLREPIGNIVTSVRELKGSETTFMCEYADETATIVEF | signal) |
| | LNRWITFSQSIISTLTGSTSGSGKPGSGEGSTKGIYIWAPLAGTSG | |
| | VLLLSLVITLYCN | |
| mIL15 | MALPVTALLLPLALLLHAARPNWVNVISDLKKIEDLIQSMHIDATL | 171 |
| | YTESDVHPSCKVTAMKCFLLELQVISLESGDASIHDTVENLIILAN | 172 (no |
| | NSLSSNGNVTESGCKECEELEEKNIKEFLQSFVHIVQMFINTSSGG | signal) |
| | GSGGGGSGGGGSGGGGSGGGSLQITCPPPMSVEHADIWVKSYSLYS | |
| | RERYICNSGFKRKAGTSSLTECVLNKATNVAHWTTPSLKCIRDPAL | |
| | VHQRPAPPSTVTTAGVTPQPESLSPSGKEPAASSPSSNNTAATTAA | |
| | IVPGSQLMPSKSPSTGTTEISSHESSHGTPSQTTAKNWELTASASH | |
| | QPPGVYPQGHSDTTVAISTSTVLLCGLSAVSLLACYLKSRQTPPLA | |
| | SVEMEAMEALPVTWGTSSRDEDLENCSHHL | |
| Anti-PDL1- | MALPVTALLLPLALLLHAARPQVQLVQSGAEVKKPGSSVKVSCKAS | 173 |
| IL2mut | GGTFSSYAISWVRQAPGQGLEWMGGIIPIFGTANYAQKFQGRVTIT | 174 (no |
| | ADESTSTAYMELSSLRSEDTAVYYCARDKWYGSAWRGDYWGQGTLV | signal) |
| | TVSSGGGGSGGGGSGGGGSQSVLTQPPSASGTPGQRVTISCSGSYS | |
| | NIGSNFVNWYQQLPGTAPKLLIHFNNQRPSGVPDRFSGSRSGTSAS | |
| | LAISGLQSEDEADYFCSTWDDSLKGPIFGGGTKLTVLGGGGSGGGG | |
| | SGGGGSAPASSSTKKTQLQLEHLLLDLQMILNGINNYKNPLLTDML | |
| | TRKFYMPKKATELKHLQCLEEELKPLEEVLNLAQSKNFHLSLREPI | |
| | GNIVTSVRELKGSETTFMCEYADETATIVEFLNRWITFSQSIISTL | |
| | T | |
| sIL15 | MALPVTALLLPLALLLHAARPNWVNVISDLKKIEDLIQSMHIDATL | 175 |
| | YTESDVHPSCKVTAMKCFLLELQVISLESGDASIHDTVENLIILAN | 176 (no |
| | NSLSSNGNVTESGCKECEELEEKNIKEFLQSFVHIVQMFINTS | signal) |
| IL2mut | MALPVTALLLPLALLLHAARPAPASSSTKKTQLQLEHLLLDLQMIL | 177 |
| | NGINNYKNPLLTDMLTRKFYMPKKATELKHLQCLEEELKPLEEVLN | 178 (no |
| | LAQSKNFHLSLREPIGNIVTSVRELKGSETTFMCEYADETATIVEF | signal) |
| | LNRWITFSQSIISTLT | |
| IL2mut-2 | MALPVTALLLPLALLLHAARPAPASSSTKKTQLQLEHLLLDLQMIL | 179 |
| | NGINNYKNPLLTDMLTRKFYMPKKATELKHLQCLEEELKPLEEVLN | 180 (no |
| | LAQSKNFHLRPRDLISNISNIVLELKGSETTFMCEYADETATIVEF | signal) |
| | LNRWITFSQSIISTLT | |

Additional information for armor polypeptides can be found, e.g., in WO2021/030633 and WO2022/159771, the relevant disclosures of each of which are incorporated by reference for the subject matter and purpose referenced herein.

In some instances, the coding sequences of the CAR constructs and the armor polypeptides may be located in a same expression cassette. The two coding sequences may be separated by a ribosome entry site (IRES) or a coding sequence for a self-cleavage peptide (e.g., P2A or T2A) so as to produce two separate polypeptides (CAR and the armor polypeptide). In other instances, two separate expression cassettes may be used to express the CAR construct and the armor polypeptide in armored CAR-T cells.

In some examples, the armor polypeptide contains a N-terminus signal peptide so that the polypeptide can be secreted from the CAR-T cells. Alternatively, the armor polypeptide may be expressed as an intracellular protein or a membrane-bound protein.

### (a) Preparation of CAR-Expressing Immune Cells

The genetically engineered immune cells disclosed herein may be prepared by introducing one or more expression cassettes encoding any of the CAR constructs disclosed herein (e.g., any of the anti-BCMA, anti-TACI, or anti-BCMA/TACI bispecific CAR constructs disclosed herein such as those provided in Table 4), optionally one or more armor polypeptides such as those disclosed herein into suitable immune cells and collecting the resultant engineered immune cells that express the CAR on cell surface.

A population of immune cells, as the starting parent cells, can be obtained from any source, such as peripheral blood mononuclear cells (PBMCs), bone marrow, or tissues such as spleen, lymph node, thymus, stem cells, or tumor tissue. A source suitable for obtaining the type of host cells desired would be evident to one of skill in the art. In some embodiments, the population of immune cells is derived from PBMCs. The type of host cells desired (e.g., T cells, NK cells, macrophages, or a combination thereof) may be expanded within the population of cells obtained by co-incubating the cells with stimulatory molecules. As a non-limiting example, anti-CD3 and anti-CD28 antibodies may be used for expansion of T cells. In some embodiments, a specific type of cells (e.g., T cells, NK cells, or macrophages) may be enriched from the immune cell population. Such enriched cell subpopulation may be expanded and/or activated in vitro prior to the genetic engineered for introduction of the CAR-encoding expression cassette and/or the armor polypeptide-encoding expression cassette (may be the same expression cassette).

To construct the immune cells that express any of the CAR polypeptides described herein (e.g., any of the anti-BCMA, anti-TACI, or anti-BCMA/TACI bispecific CAR constructs disclosed herein such as those provided in Table 4), optionally one or more armor polypeptides such as those disclosed herein (e.g., those provided in Table 11), expression vectors for stable or transient expression of the CAR polypeptide and optionally the armor polypeptide may be created via conventional methods and introduced into immune host cells. For example, nucleic acids encoding the CAR polypeptides and optionally the armor polypeptide may be cloned into one or more suitable expression vector(s), such as a viral vector(s) in operable linkage to a suitable promoter. Non-limiting examples of useful vectors of the disclosure include viral vectors such as, e.g., retroviral vectors including gamma retroviral vectors, adeno-associated virus vectors (AAV vectors), and lentiviral vectors. The nucleic acids and the vector may be contacted, under suitable conditions, with a restriction enzyme to create complementary ends on each molecule that can pair with each other and be joined with a ligase. Alternatively, synthetic nucleic acid linkers can be ligated to the termini of the nucleic acid encoding the CAR polypeptides, and optionally the armor polypeptide. The synthetic linkers may contain nucleic acid sequences that correspond to a particular restriction site in the vector. The selection of expression vectors/plasmids/viral vectors would depend on the type of host cells for expression of the CAR polypeptides and optionally the armor polypeptide but should be suitable for integration and replication in eukaryotic cells. Any of such nucleic acids encoding the CAR and optionally the armor polypeptide and expression vectors comprising such are also within the scope of the present disclosure.

A variety of promoters can be used for expression of the CAR polypeptides and optionally the armor polypeptide described herein, including, without limitation, cytomegalovirus (CMV) intermediate early promoter, a viral LTR such as the Rous sarcoma virus LTR, HIV-LTR, HTLV-1 LTR, the simian virus 40 (SV40) early promoter, or herpes simplex tk virus promoter. Additional promoters for expression of the CAR polypeptides and optionally the armor polypeptide include any constitutively active promoter in an immune cell. Alternatively, any regulatable promoter may be used, such that its expression can be modulated within an immune cell. In some embodiments, the promoter can be the pEF1α promoter.

Additionally, the vector may contain, for example, some or all of the following: a selectable marker gene, such as the neomycin gene or the kanamycin gene for selection of stable or transient transfectants in host cells; enhancer/promoter sequences from the immediate early gene of human CMV for high levels of transcription; transcription termination and RNA processing signals from SV40 for mRNA stability; SV40 polyomavirus origins of replication and ColE1 for proper episomal replication; internal ribosome binding sites (IRESes), versatile multiple cloning sites; T7 and SP6 RNA promoters for in vitro transcription of sense and antisense RNA; a “suicide switch” or “suicide gene” which when triggered causes cells carrying the vector to die (e.g., HSV thymidine kinase or an inducible caspase such as iCasp9), and reporter gene for assessing expression of the CAR polypeptide.

In one specific embodiment, such vectors may also include a suicide gene. As used herein, the term “suicide gene” refers to a gene that causes the cell expressing the suicide gene to die. The suicide gene can be a gene that confers sensitivity to an agent, e.g., a drug, upon the cell in which the gene is expressed, and causes the cell to die when the cell is contacted with or exposed to the agent. Suicide genes are known in the art (see, for example, Suicide Gene Therapy: Methods and Reviews, Springer, Caroline J. (Cancer Research UK Centre for Cancer Therapeutics at the Institute of Cancer Research, Sutton, Surrey, UK), Humana Press, 2004) and include, for example, the Herpes Simplex Virus (HSV) thymidine kinase (TK) gene, cytosine deaminase, purine nucleoside phosphorylase, nitroreductase, and caspases such as caspase 8.

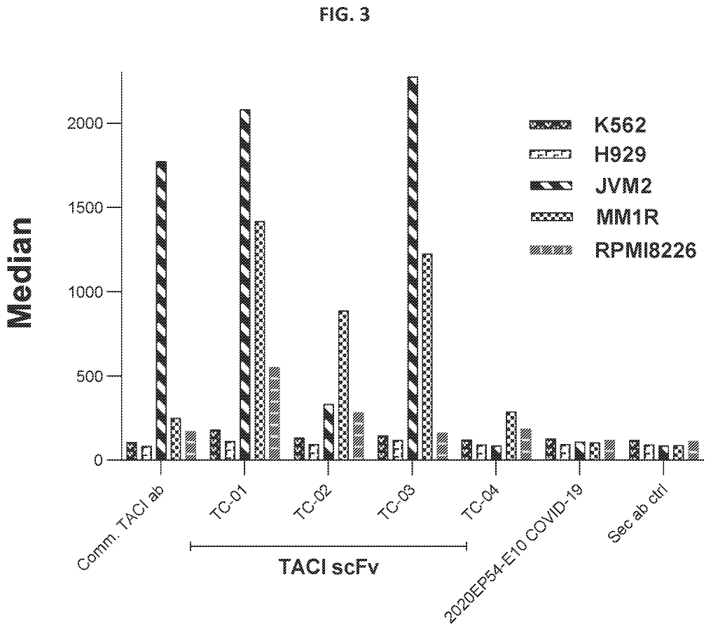

The nucleic acid disclosed herein may comprise two coding sequences, one for any of the CAR constructs disclosed herein (e.g., any of the anti-BCMA, anti-TACI, or anti-BCMA/TACI bispecific CAR constructs disclosed herein such as those provided in Table 4) and the other for the armor polypeptide. The two coding sequences may be configured such that the polypeptides encoded by the two coding sequences can be expressed as independent (and physically separate) polypeptides. To achieve this goal, the nucleic acid described herein may contain a third nucleotide sequence located between the first and second coding sequences. This third nucleotide sequence may, for example, encode a ribosomal skipping site. A ribosomal skipping site is a sequence that impairs normal peptide bond formation. This mechanism results in the translation of additional open reading frames from one messenger RNA. This third nucleotide sequence may, for example, encode a self-cleavage peptide such as P2A, T2A, or F2A peptide (see, for example, Kim et al., PLoS One. 2011; 6(4):e18556). See also FIG. 3.

Any of the vectors comprising a nucleic acid sequence that encodes an ACTR polypeptide and optionally an armor polypeptide described herein is also within the scope of the present disclosure.

Such a vector, or the sequence encoding a CAR polypeptide and optionally the armor polypeptide contained therein, may be delivered into host cells such as host immune cells (e.g., T cells, NK cells, or macrophages) by any suitable method. Methods of delivering vectors to immune cells are well known in the art and may include DNA electroporation, RNA electroporation, transfection using reagents such as liposomes, or viral transduction (e.g., retroviral transduction such as lentiviral transduction).

Following introduction into the host cells a vector encoding any of the CAR polypeptides provided herein (e.g., anti-BCMA, anti-TACI, or anti-BCMA/TACI bispecific CAR constructs disclosed herein such as those provided in Table 4), optionally the armor polypeptide as also provided herein, the cells may be cultured under conditions that allow for expression of the CAR polypeptide and optionally the armor polypeptide. When expression of the CAR polypeptide and/or the armor polypeptide is regulated by a regulatable promoter, the host cells may be cultured in conditions wherein the regulatable promoter is activated. In some embodiments, the promoter is an inducible promoter and the immune cells are cultured in the presence of the inducing molecule or in conditions in which the inducing molecule is produced. Determining whether the CAR polypeptide and/or the armor polypeptide is expressed will be evident to one of skill in the art and may be assessed by any known method, for example, detection of the CAR polypeptide-encoding and/or armor polypeptide-encoding mRNA by quantitative reverse transcriptase PCR (qRT-PCR) or detection of the CAR or armor polypeptide protein by methods including Western blotting, fluorescence microscopy, and flow cytometry. Alternatively, expression of functional CAR may be determined by binding activity and/or CTL activity against cells expressing the target antigen, e.g., BCMA and/or TACI.

Methods for preparing host cells expressing any of the CAR polypeptides, and optionally an armor polypeptide, as described herein, may also comprise activating the host cells ex vivo. Activating a host cell means stimulating a host cell into an activated state in which the cell may be able to perform effector functions. Methods of activating a host cell will depend on the type of host cell used for expression of the CAR polypeptides and optionally the armor polypeptide. For example, T cells may be activated ex vivo in the presence of one or more molecules including, but not limited to: an anti-CD3 antibody, an anti-CD28 antibody, IL-2, and/or phytohemoagglutinin. In other examples, NK cells may be activated ex vivo in the presence of one or molecules such as a 4-1BB ligand, an anti-4-1BB antibody, IL-15, an anti-IL-15 receptor antibody, IL-2, IL12, IL-21, and/or K562 cells. In some embodiments, the host cells expressing any of the CAR polypeptides (CAR-expressing cells), and optionally the armor polypeptide (armored CAR cells) described herein are activated ex vivo prior to administration to a subject. Determining whether a host cell is activated will be evident to one of skill in the art and may include assessing expression of one or more cell surface markers associated with cell activation, expression or secretion of cytokines, and cell morphology.

Methods for preparing host cells expressing any of the CAR polypeptides, and optionally the armor polypeptide, described herein may comprise expanding the host cells ex vivo. Expanding host cells may involve any method that results in an increase in the number of cells expressing CAR polypeptides and optionally the armor polypeptide, for example, allowing the host cells to proliferate or stimulating the host cells to proliferate. Methods for stimulating expansion of host cells will depend on the type of host cell used for expression of the CAR polypeptides, and optionally the armor polypeptide, and will be evident to one of skill in the art. In some embodiments, the host cells expressing any of the CAR polypeptides, optionally the armor polypeptide, described herein are expanded ex vivo prior to administration to a subject.

In some embodiments, the host cells expressing the CAR polypeptides and optionally the armor polypeptide are expanded and activated ex vivo prior to administration of the cells to the subject. Host cell activation and expansion may be used to allow integration of a viral vector into the genome and expression of the gene encoding a CAR polypeptide and optionally the armor polypeptide as described herein. If mRNA electroporation is used, no activation and/or expansion may be required, although electroporation may be more effective when performed on activated cells.

In some instances, a CAR polypeptide and/or an armor polypeptide is transiently expressed in a suitable host cell (e.g., for 3-5 days). Transient expression may be advantageous if there is a potential toxicity and should be helpful in initial phases of clinical testing for possible side effects.

### (b) Pharmaceutical Compositions

Any of the genetically engineered immune cells expressing a CAR and optionally an armor polypeptide as disclosed herein (e.g., any of the anti-BCMA, anti-TACI, or anti-BCMA/TACI bispecific CAR constructs such as those provided in Table 4 above) may be mixed with a pharmaceutically acceptable carrier to form a pharmaceutical composition, which is also within the scope of the present disclosure.

The phrase “pharmaceutically acceptable”, as used in connection with compositions of the present disclosure, refers to molecular entities and other ingredients of such compositions that are physiologically tolerable and do not typically produce untoward reactions when administered to a mammal (e.g., a human). Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans. “Acceptable” means that the carrier is compatible with the active ingredient of the composition (e.g., the nucleic acids, vectors, cells, or therapeutic antibodies) and does not negatively affect the subject to which the composition(s) are administered. Any of the pharmaceutical compositions to be used in the present methods can comprise pharmaceutically acceptable carriers, excipients, or stabilizers in the form of lyophilized formations or aqueous solutions.

Pharmaceutically acceptable carriers, including buffers, are well known in the art, and may comprise phosphate, citrate, and other organic acids; antioxidants including ascorbic acid and methionine; preservatives; low molecular weight polypeptides; proteins, such as serum albumin, gelatin, or immunoglobulins; amino acids; hydrophobic polymers; monosaccharides; disaccharides; and other carbohydrates; metal complexes; and/or non-ionic surfactants. See, e.g., Remington: The Science and Practice of Pharmacy 20^{th} Ed. (2000) Lippincott Williams and Wilkins, Ed. K. E. Hoover.

For examples of additional useful agents, see also Physician's Desk Reference, 59.sup.th edition, (2005), Thomson P D R, Montvale N.J.; Gennaro et al., Eds. Remington's The Science and Practice of Pharmacy 20th edition, (2000), Lippincott Williams and Wilkins, Baltimore Md.; Braunwald et al., Eds. Harrison's Principles of Internal Medicine, 15.sup.th edition, (2001), McGraw Hill, NY; Berkow et al., Eds. The Merck Manual of Diagnosis and Therapy, (1992), Merck Research Laboratories, Rahway N.J.

### IV. Therapeutic Applications

Any of the genetically engineered immune cells (e.g., T cells, NK cells, or macrophages) expressing a CAR as disclosed herein (e.g., any of the anti-BCMA, anti-TACI, or anti-BCMA/TACI bispecific CAR constructs provided in Table 4), and optionally an armor polypeptide as also disclosed herein (e.g., those provided in Table 11) may be used for therapeutic purposes, for example, to eliminate undesired cells expressing BCMA and/or TACI. In some examples, the genetically engineered immune cells are armored CAR-T cells expressing any of the anti-BCMA, anti-TACI, or anti-BCMA/TACI bispecific CAR constructs such as those provided in Table 4 above, together with an armor polypeptide such as those provided in Table 11 above.

To practice the method described herein, an effective amount of the immune cells (NK cells, T lymphocytes, or macrophages) expressing any of the CAR described herein (e.g., any of the anti-BCMA, anti-TACI, or anti-BCMA/TACI bispecific CAR constructs such as those provided in Table 4 above), and optionally an armor polypeptide (e.g., those provided in Table 11 above) or pharmaceutical compositions thereof may be administered to a subject in need of the treatment via a suitable route, such as intravenous administration. As used herein, an effective amount refers to the amount of the respective agent (e.g., the NK cells, T lymphocytes or macrophages expressing the CAR and optionally the armor polypeptide) that upon administration confers a therapeutic effect on the subject. Determination of whether an amount of the cells or compositions described herein achieved the therapeutic effect would be evident to one of skill in the art. Effective amounts vary, as recognized by those skilled in the art, depending on the particular condition being treated, the severity of the condition, the individual patient parameters including age, physical condition, size, gender, sex, and weight, the duration of the treatment, the nature of concurrent therapy (if any), the specific route of administration and like factors within the knowledge and expertise of the health practitioner. In some embodiments, the effective amount alleviates, relieves, ameliorates, improves, reduces the symptoms, or delays the progression of any disease or disorder in the subject. In some embodiments, the subject is a human. In some embodiments, the subject in need of treatment is a human cancer patient.

As used herein, the term “therapeutically effective” applied to dose or amount refers to that quantity of a compound or pharmaceutical composition that is sufficient to result in a desired activity upon administration to a subject in need thereof. Note that when a combination of active ingredients is administered, the effective amount of the combination may or may not include amounts of each ingredient that would have been effective if administered individually. Within the context of the present disclosure, the term “therapeutically effective” refers to that quantity of a compound or pharmaceutical composition that is sufficient to delay the manifestation, arrest the progression, relieve or alleviate at least one symptom of a disorder treated by the methods of the present disclosure.

In some embodiments, the methods of the disclosure may be used for eliminating or inhibiting disease cells expressing BCMA and/or TAC. Accordingly, any of the immune cells disclosed herein may be used for treating a disease associated with BCMA⁺ and/or TACI⁺ disease cells, such as BCMA⁺ and/or TACI⁺ cancer cells. The method disclosed herein may be used for treating a cancer involving BCMA⁺ and/or TACI⁺ cancer cells, for example, multiple myeloma, lung cancer, gastric cancer, breast cancer, testis cancer, etc.

In some embodiments, an effective amount of any of the genetically engineered immune cells express a CAR as disclosed herein (e.g., any of the anti-BCMA, anti-TACI, or anti-BCMA/TACI bispecific CAR constructs provided in Table 4), and optionally an armor polypeptide (e.g., those provided in Table 11) may be given to a subject in need of the treatment via a suitable route, for example, intravenous infusion. The subject may be a human patient having a disease associated with BMCA⁺ and/or TACI⁺ disease cells, such as BCMA⁺ and/or TACI⁺ cancer cells. In some instances, the human patient has a cancer involving BCMA⁺ and/or TACI⁺ cancer cells. In some instances, the human patient may have multiple myeloma, lung cancer, gastric cancer, breast cancer, or testis cancer.

In some embodiments, the immune cells (e.g., NK and/or T cells) for use in the treatment disclosed herein may be autologous to the subject, i.e., the immune cells may be obtained from the subject in need of the treatment, genetically engineered for expression of the CAR polypeptides, and then administered to the same subject. In one specific embodiment, prior to re-introduction into the subject, the autologous immune cells (e.g., T lymphocytes, NK cells, or macrophages) are activated and/or expanded ex vivo. Administration of autologous cells to a subject may result in reduced rejection of the host cells as compared to administration of non-autologous cells.

Alternatively, the genetically engineered immune cells (e.g., T cells, NK cells, or macrophages) can be allogeneic cells, i.e., the cells are obtained from a first subject, genetically engineered for expression of the CAR polypeptide, and administered to a second subject that is different from the first subject but of the same species. For example, allogeneic immune cells may be derived from a human donor and administered to a human recipient who is different from the donor. In a specific embodiment, the T lymphocytes are allogeneic T lymphocytes, in which the expression of the endogenous T cell receptor has been inhibited or eliminated. In one specific embodiment, prior to introduction into the subject, the allogeneic T lymphocytes are activated and/or expanded ex vivo. T lymphocytes can be activated by any method known in the art, e.g., in the presence of anti-CD3/CD28, IL-2, and/or phytohemoagglutinin.

NK cells can be activated by any method known in the art, e.g., in the presence of one or more agents selected from the group consisting of CD137 ligand protein, CD137 antibody, IL-15 protein, IL-15 receptor antibody, IL-2 protein, IL-12 protein, IL-21 protein, and K562 cell line. See, e.g., U.S. Pat. Nos. 7,435,596 and 8,026,097 for the description of useful methods for expanding NK cells. For example, NK cells used in the methods of the disclosure may be preferentially expanded by exposure to cells that lack or poorly express major histocompatibility complex I and/or II molecules and which have been genetically modified to express membrane bound IL-15 and 4-1BB ligand (CDI37L). Such cell lines include, but are not necessarily limited to, K562 [ATCC, CCL 243; Lozzio et al., Blood 45(3): 321-334 (1975); Klein et al., Int. J. Cancer 18: 421-431 (1976)], and the Wilms tumor cell line HFWT (Fehniger et al., Int Rev Immunol 20(3-4):503-534 (2001); Harada H, et al., Exp Hematol 32(7):614-621 (2004)), the uterine endometrium tumor cell line HHUA, the melanoma cell line HMV-II, the hepatoblastoma cell line HuH-6, the lung small cell carcinoma cell lines Lu-130 and Lu-134-A, the neuroblastoma cell lines NB 19 and N1369, the embryonal carcinoma cell line from testis NEC 14, the cervix carcinoma cell line TCO-2, and the bone marrow-metastasized neuroblastoma cell line TNB 1 [Harada, et al., Jpn. J. Cancer Res 93: 313-319 (2002)]. Preferably the cell line used lacks or poorly expresses both MHC I and II molecules, such as the K562 and HFWT cell lines. A solid support may be used instead of a cell line. Such support should preferably have attached on its surface at least one molecule capable of binding to NK cells and inducing a primary activation event and/or a proliferative response or capable of binding a molecule having such an affect thereby acting as a scaffold. The support may have attached to its surface the CD137 ligand protein, a CD137 antibody, the IL-15 protein or an IL-15 receptor antibody. Preferably, the support will have IL-15 receptor antibody and CD137 antibody bound on its surface.

In accordance with the present disclosure, patients can be treated by infusing therapeutically effective doses of immune cells such as T lymphocytes or NK cells expressing a CAR polypeptide such as an anti-BCMA monospecific CAR, an anti-TACI monospecific CAR, or an anti-BCMA/anti-TACI bispecific CAR as listed in Table 4 above, and optionally an armor polypeptide as listed in Table 11 in the range of about 10⁵ to 10⁹ CAR+ cells to a patient. The infusion can be repeated as often and as many times as the patient can tolerate until the desired response is achieved. The appropriate infusion dose and schedule will vary from patient to patient but can be determined by the treating physician for a particular patient. In some examples, initial doses of approximately 10⁶ cells/Kg can be infused, escalating to 10⁸ or more cells/kg.

The particular dosage regimen, i.e., dose, timing and repetition, used in the method described herein will depend on the particular subject and that subject's medical history. The appropriate dosage of the CAR-expressing immune cells (e.g., armored CAR-T cells) used will depend on the type of cancer to be treated, the severity and course of the disease, previous therapy, the patient's clinical history and response to the immune cell therapy, and the discretion of the attending physician.

In some embodiments, the genetically engineered immune cells (e.g., armored CAR-T cells) expressing any of the CAR constructs disclosed herein (e.g., the anti-BCMA CAR, the anti-TACI CAR, or the anti-BCMA/TACI bispecific CAR) may be utilized in conjunction with other types of therapy for cancer, such as chemotherapy, surgery, radiation, gene therapy, and so forth. Such therapies can be administered simultaneously or sequentially (in any order) with the immunotherapy according to the present disclosure. When co-administered with an additional therapeutic agent, suitable therapeutically effective dosages for each agent may be lowered due to the additive action or synergy.

### V. Kits for Therapeutic Applications

The present disclosure also provides kits for use of the genetically engineered immune cells (e.g., T lymphocytes, NK cells, or macrophages) expressing anti-BCMA CAR, anti-TACI CAR, or anti-BCMA/anti-TACI bispecific CAR, and optionally an armor polypeptide as described herein. See, e.g., Table 11. Such kits may include one or more containers comprising the genetically engineered immune cells, which may be formulated in a pharmaceutical composition further comprising a pharmaceutically acceptable carrier.

In some embodiments, the kit described herein comprises genetically engineered immune cells, which may be expanded in vitro. The immune cells may express any of the CAR disclosed herein, for example, any of the anti-BCMA CARs, anti-TACI CARs, and anti-BCMA/TACI bispecific CARs such as those provided in Table 4 above. The immune cells may be armored CAR-T or CAR-NK cells, which further express an armor polypeptide.

In some embodiments, the kit can additionally comprise instructions for use in any of the methods described herein. The included instructions may comprise a description of administration of the genetically engineered immune cells disclosed herein to achieve the intended activity, e.g., eliminating the target disease cells such as cancer cells expressing BCMA, TACI, or both, in a subject. The kit may further comprise a description of selecting a subject suitable for treatment based on identifying whether the subject is in need of the treatment.

The instructions relating to the use of the genetically engineered immune cells described herein generally include information as to dosage, dosing schedule, and route of administration for the intended treatment. The containers may be unit doses, bulk packages (e.g., multi-dose packages) or sub-unit doses. Instructions supplied in the kits of the disclosure are typically written instructions on a label or package insert. The label or package insert indicates that the genetically engineered immune cells are used for treating, delaying the onset, and/or alleviating a disease or disorder associated with BCMA and/or TACI positive disease cells in a subject.

The kits provided herein are in suitable packaging. Suitable packaging includes, but is not limited to, vials, bottles, jars, flexible packaging, and the like. Also contemplated are packages for use in combination with a specific device, such as an inhaler, nasal administration device, or an infusion device. A kit may have a sterile access port (for example, the container may be an intravenous solution bag or a vial having a stopper pierceable by a hypodermic injection needle). The container may also have a sterile access port.

Kits optionally may provide additional components such as buffers and interpretive information. Normally, the kit comprises a container and a label or package insert(s) on or associated with the container. In some embodiment, the disclosure provides articles of manufacture comprising contents of the kits described above.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. (1985»; Transcription and Translation (B. D. Hames & S. J. Higgins, eds. (1984»; Animal Cell Culture (R. I. Freshney, ed. (1986»; Immobilized Cells and Enzymes (IRL Press, (1986»; and B. Perbal, A practical Guide To Molecular Cloning (1984); F. M. Ausubel et al. (eds.).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### Example 1. Generation and Characterization of Anti-BCMA Antibodies

This example illustrates identification and characterization of exemplary anti-BCMA antibodies.

Anti BCMA scFv Antibody Screening by mRNA Display

mRNA display technology was used for the identification of BCMA binders from 10¹²⁻¹³ natural human scFv library. Briefly, the scFv DNA library was first transcribed into mRNA library and then translated into mRNA-scFv fusion library by covalent coupling through a puromycin linker, similar to the reported procedure (U.S. Pat. No. 6,258,558B1, relevant disclosures of which are incorporated by reference for the subject matter and purpose referenced herein). The fusion library was first counter selected with human IgGs (negative proteins) multiple times to remove non-specific binders, followed by selection against recombinant BCMA-Fc fusion protein and captured on Protein G magnetic beads. Binders were eluted off and then enriched by PCR amplification with library specific oligos. At round 5-6, the scFv library was selected against the BCMA/HEK293 cell line. Total of 6 rounds of selections executed to generate highly enriched BCMA binding pools for screening.

After 6 rounds of selections, the BCMA enriched scFv library was cloned into bacterial periplasmic expression vector pET22b and transformed into TOP 10 competent cells. Each of the scFv molecule was engineered to have a C-terminal flag and 6×His tag for purification and assay detection. Clones from TOP 10 cells were pooled and the miniprep DNA were prepared and subsequently transformed into bacterial Rosetta II strain for expression. Single clone was picked, grown and induced with 0.1 mM IPTG in 96 well plate for expression. The supernatant was collected after 16-24 hours induction at 30° C. for assays to identify anti-BCMA antibodies.

BCMA binding screening FACS was developed for the identification of individual anti-BCMA antibodies. Briefly, 100,000 BCMA/CHOK1 cells and CHOK1 cells were seeded in 96-well cell culture plate respectively. The cells and culture media were chilled at 4 C and spun down at 1200 rpm for 6 mins. The bacterial supernatant was diluted to final 30% with cell culture media and added and BCMA/CHOK1 cells. The plate was incubated at 4 C with shaking for 1 hour. The cells were spun down as above and supernatant removed. The plate was then washed 200 ul with full media at 4 C. 100 ul of 1:250 pre-diluted AF-647 conjugated Anti-HIS tag antibody was added to the cells. The plate was incubated at 4 C in dark with shaking for 30 mins. The plate was washed twice as described and cells were reconstituted in 200 ul of full media. The cells were mixed well subjected to read on Attune NxT cytometer. Analysis was performed using the Attune NxT software plotting the overlaying the histogram of anti-BCMA scFv binding onto both negative and target cell lines.

The selective anti-BCMA scFv clones were picked from a glycerol stock plate and grown overnight into a 5 mL culture in a Thomson 24-well plate with a breathable membrane. This culture, and all subsequent cultures described below were grown at 37° C. and shaking at 225 RPM in Terrific Broth Complete plus 100 μg/mL carbenicillin and 34 μg/mL chloramphenicol, with 1:5,000 dilution of antifoam-204 also added, unless specified otherwise. This overnight starter culture was then used to inoculate the larger culture, 1:100 dilution of starter culture into the designated production culture and grown until OD600 was between 0.5-0.8. At this point, the culture was induced with a final concentration of IPTG at 0.1 mM and incubated over night at 30° C. The following day, the cultures were spun for 30 min at 5,000×g, to pellet the cells and then the supernatant was filter sterilized through a 0.2 um sterilizing PES membrane.

For the purification, 3 uL GE Ni Sepharose Excel resin per 1 mL of filtered supernatant was used. Disposable 10 mL or 20 mL BioRad Econo-Pac columns were used. The resin was equilibrated with at least 20 column volume (CV) buffer A (1×PBS, pH7.4 with extra NaCl added to 500 mM). The filter sterilized supernatant was purified by gravity flow by either controlling the flow to 1 mL/min or was poured over two times, over same packed resin bed. The column was then washed with the following buffers: 10 CV buffer A, 20 CV buffer B (1×PBS, pH7.4 with extra NaCl to 500 mM, and 30 mM imidazole). The two Detox buffers were used to remove endotoxin as optional step if needed. For 250 mL expression culture purifications, antibody bound column was washed sequentially with 20 CV buffer C (1×PBS pH7.4 with extra NaCl to 500 mM, 1% Tx114), 20 CV buffer D (1×PBS pH7.4 with extra NaCl to 500 mM, 1% Tx100+0.2% TNBP) and 40 CV buffer E (1×PBS pH7.4 with extra NaCl to 500 mM). The protein was eluted with Eluting buffer F (1×PBS pH7.4 with extra NaCl to 500 mM, and 500 mM imidazole) in a total of six fractions (0.5 CV pre elute, 5×1 CV elute). Fractions were run on a Bradford assay (100 ul diluted Bradford solution+10 ul sample). Fractions with bright blue color were pooled. Protein concentration was measured by A280 extension coefficient. SDS-PAGE gel to analyze the purity of the purified antibodies.

Characterization of Exemplary Anti BCMA scFv Antibodies

Exemplary anti-BCMA antibodies in scFv format isolated by mRNA library screening as disclosed above were analyzed to determining their antigen binding affinity. Structural information of the exemplary anti-BCMA antibodies is provided in Table 1 above.

### (a) BCMA Binding Activity Via ELISA

An ELISA assay was developed to determine the EC₅₀ of anti-BCMA antibodies. Briefly, 384 well plate was immobilized with anti-human Fc antibody at final concentration of 2 μg/mL in 1×PBS in total volume of 25 uL per well. The plate was incubated overnight at 4° C. followed by blocking with 80 μL of superblock per well for 1 hour. Human BCMA-Fc was captured through immobilized anti-hFc antibody. Purified anti-BCMA scFvs were 2-fold serial titrated from 200 nM. 25 μL of diluted scFv was added to human BCMA immobilized wells and incubated for 1 hour with shaking. The BCMA binding was detected by adding 25 μL of anti-Flag HRP diluted at 1:5000 in 1×PBST. In between each step, the plate was washed 3 times with 1×PBST in a plate washer. The plate was then developed with 20 μL of TMB substrate for 5 mins and stopped by adding 20 μL of 2 N sulfuric acid. The plate was read at OD450 nm Biotek plate reader and then plotted in Prism 8.1 software. EC₅₀ values were calculated and showed in Table 5 below.

TABLE 5
BCMA-Binding Activity of Exemplary Anti-
BCMA Antibodies Determined by ELISA

| Antibodies (in scFv format) | EC₅₀ (nM) |
|---|---|
| BC-01 | 0.938 |
| BC-02 | 1.916 |
| BC-03 | 2.319 |
| BC-04 | 1.964 |
| BC-05 | 0.5357 |
| BC-06 | 0.6201 |
| BC-07 | 0.8016 |
| BC-08 | 0.2989 |
| BC-09 | 0.5968 |

### (b) BCMA Binding Activity Via Surface Plasmon Resonance (SPR)

Kinetic analysis of anti-BCMA scFvs have been assessed by SPR technology with Biacore T200. The assay was run with Biacore T200 control software version 2.0. Protein A sensor chip was used to capture Fc fusion protein in the assay. For each cycle, 1 μg/mL of human BCMA-Fc protein was captured for 60 seconds at flow rate of 10 ul/min on flow cell 2 in 1×HBSP buffer on Protein A sensor chip. 2-fold serial diluted HIS tag purified anti-BCMA scFv was injected onto both reference flow cell 1 and BCMA-Fc captured flow cell 2 for 150 seconds at flow rate of 30 ul/min followed by wash for 300 seconds. The flow cells were then regenerated with Glycine pH2 buffer (GE) for 30 seconds at flow rate of 30 ul/mins. 8 concentration points from 300-0 nM was assayed per anti-BCMA scFv in a 96 well plate. The kinetics of scFvs binding to BCMA protein was analyzed with Biacore T200 evaluation software version 3.0. The specific binding response unit was derived from subtraction of binding to reference flow cell 1 from BCMA captured flow cell 2. The Kon, Koff and KD values were calculated for exemplary anti-BCMA antibodies (in scFv format) showed in the Table 6 below.

TABLE 6
BCMA-Binding Activities of Exemplary Anti-
BCMA Antibodies Determined by SPR

| Antibodies | ka (1/Ms) | kd (1/s) | KD (M) |
|---|---|---|---|
| BC-01 | 1.44E+05 | 4.31E−04 | 3.00E−09 |
| BC-02 | 9.16E+04 | 1.18E−03 | 1.29E−08 |
| BC-03 | 3.02E+05 | 3.59E−03 | 1.19E−08 |
| BC-04 | 1.14E+05 | 8.74E−04 | 7.68E−09 |
| BC-05 | 6.24E+04 | 6.46E−04 | 1.04E−08 |
| BC-06 | 3.19E+05 | 2.51E−03 | 7.87E−09 |
| BC-07 | 2.26E+05 | 5.92E−03 | 2.62E−08 |
| BC-08 | 1.78E+06 | 1.16E−03 | 6.53E−10 |
| BC-09 | 2.08E+05 | 4.07E−03 | 1.96E−08 |

(c) scFv Antibody Binding to Cell Surface BCMA Via FACS

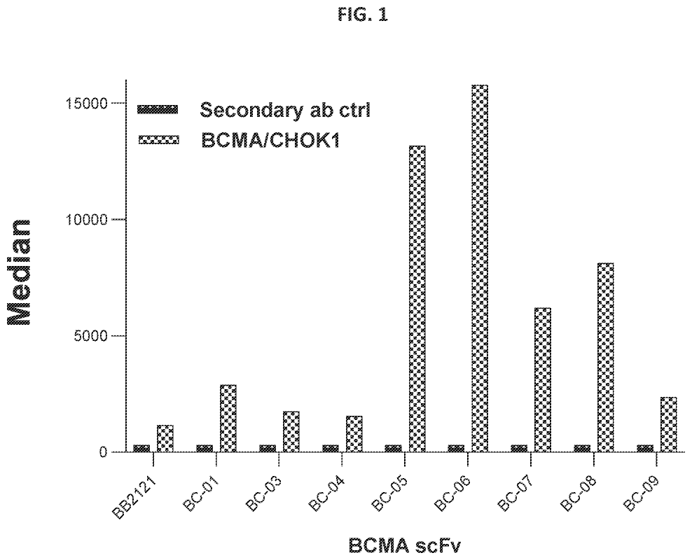

To determine the binding selectivity and affinity of anti-BCMA scFvs bind to BCMA expressing cells, 200 nM of purified anti-BCMA scFv antibodies were diluted in full medium and incubated with recombinant BCMA/CHOK1 and CHOK1 cells in 96 wells plate on ice for 1 hour. Cells were spun down at 1200 rpm for 6 minutes at 4° C. to remove primary antibodies. Cells were then washed once with 200 μL of full medium per well. Samples were detected with premixed anti-His Biotin Streptavidin Alexa fluor 647 by adding 100 μL of diluted secondary antibody and incubated at 4° C. for 30 minutes in the dark. Samples were spun down at 1200 rpm for 5 minutes at 4° C. and washed twice with 200 μL of 1×PBS per well. Reconstituted samples in 200 μL of 1×PBS and read on Attune NxT cytometer. Analysis was done by Attune NxT software plotting the overlaying the histogram of anti-BCMA scFvs binding onto both negative and target cell lines. The anti-BCMA scFvs showed selective binding to BCMA/CHOK1 cells and not to CHOK1 parental cells (FIG. 1). The BCMA cell binding affinity for exemplary anti-BCMA antibodies (in scFv format) was also generated with serial diluted scFvs as described above. EC₅₀ was calculated and showed in the Table 7 below.

TABLE 7
BCMA-Binding Activities of Exemplary Anti-
BCMA Antibodies Determined by FACS

| ScFvs | EC50 (nM) |
|---|---|
| BC-01 | 18.91 |
| BC-02 | 83.99 |
| BC-03 | 16.06 |
| BC-04 | 69.67 |
| BC-05 | 4.913 |
| BC-06 | 1.901 |
| BC-07 | 13.2 |
| BC-08 | 0.9998 |
| BC-09 | 5.278 |

### (d) Anti-BCMA Antibody Binding to Endogenous BCMA-Expressing Cell Lines

To further characterize the BCMA and TACI expression in recombinant and endogenous cell lines, quantification FACS assay was performed using Bangs Laboratories Inc Quantum Alexa Fluor 647 MESF microsphere beads for standard calibration following manufacture's protocol. The parental CHOK1 cell line showed non-detectable BCMA and TACI expression. There is no TACI expression in H929 and U226B1 cell lines. The BCMA showed 4-6-fold higher expression than TACI MM1.R, MM1.S and RPMI8226 multiple myeloma cell lines. The BCMA and TACI receptor copy number are summarized in Table 8.

TABLE 8
Copy Numbers of BCMA and TACI in Various Cell Lines

| Cell Lines | BCMA receptors | TACI receptors |
|---|---|---|
| CHOK1 | 0 | 0 |
| CHOK1-BCMA | 339834 | 0 |
| H929 | 197350 | 0 |
| MM1.S | 29894 | 6409 |
| RPMI8226 | 8532 | 1386 |
| U266B1 | 42257 | 0 |
| MM1.R | 52419 | 12578 |
| JVM-2 | 18057 | 41088 |

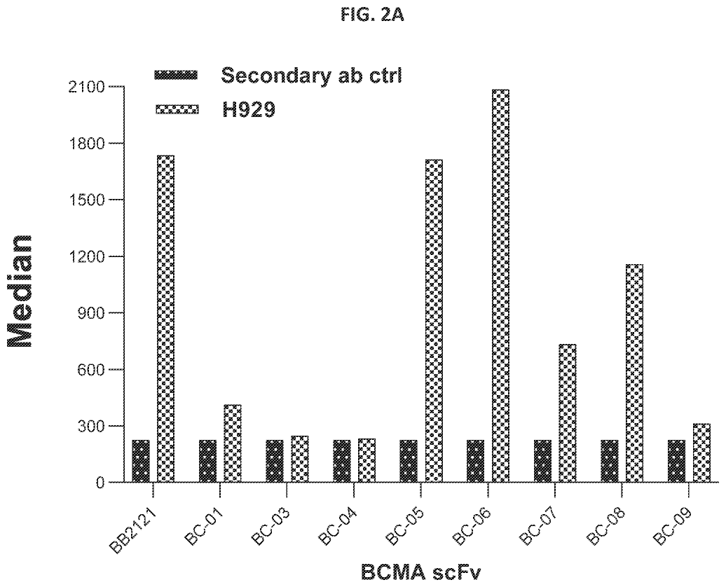

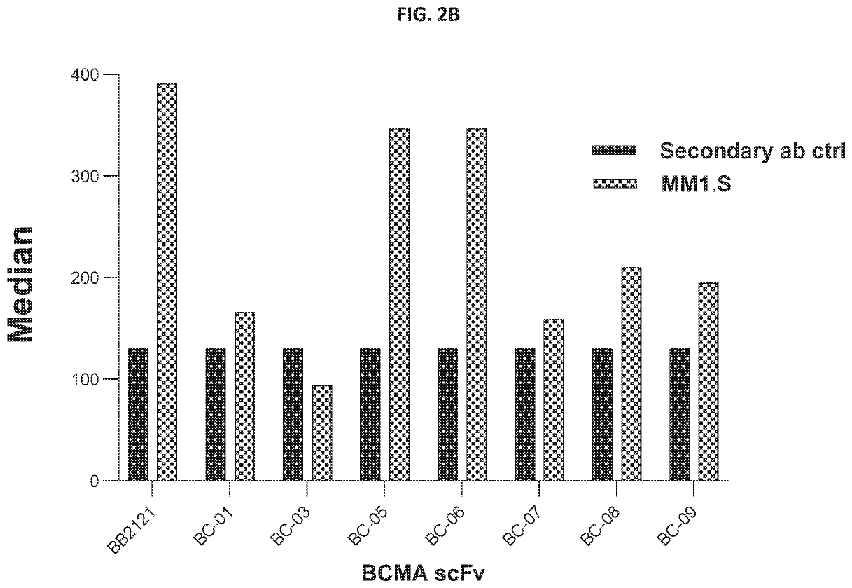

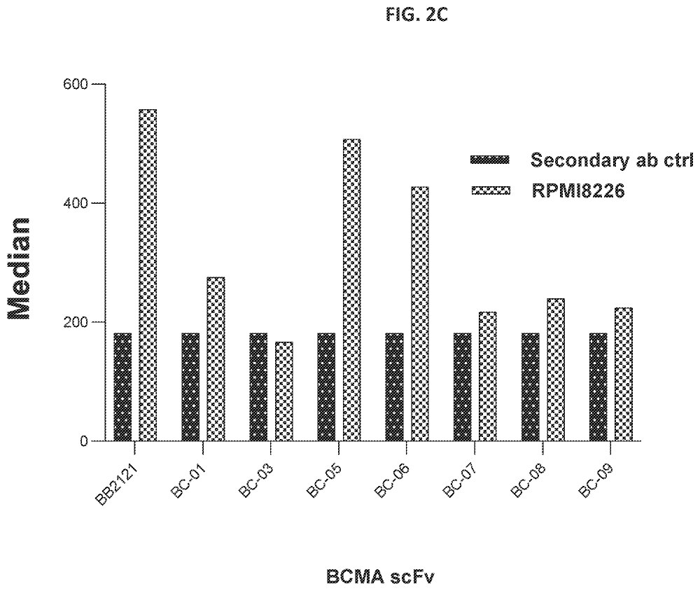

The binding of anti-BCMA antibodies (scFvs) to endogenous BCMA were assessed in a FACS binding assay as described above. 200 nM of anti-BCMA scFvs were tested with negative cell line, H929, MM1.S and RPMI8226. These exemplary antibodies demonstrated similar binding patten with different cell lines and the binding intensity correlated with BCMA receptor number on each cell line (FIGS. 2A-2C).

The binding activity of exemplary anti-BCMA clone BC-06 (in scFv format; see Table 1 above) to multiple myeloma cell lines (BCMA+ or BCMA−) were examined and the EC₅₀ values are provided in Table 9 below.

TABLE 9
EC₅₀ Values of Clone BC-06 to Multiple
Myeloma and Control Cell Lines

| Cell Line | MM1R | H929 | K562 |
|---|---|---|---|
| EC₅₀ (nM) | 0.74 | 2.85 | ND |

### Example 2. Generation and Characterization of Anti-TACI Antibodies

This example illustrates identification and characterization of exemplary anti-TACI antibodies.

Anti TACI scFv Antibody Screening by mRNA Display

mRNA display technology as described in Example 1 above was used for the identification of TACI binders from 10¹²⁻¹³ natural human scFv libraries, following the procedures described in Example 1 above.

Characterization of Exemplary Anti TACI scFv Antibodies

Exemplary anti-TACI antibodies in scFv format isolated by mRNA library screening as disclosed above were analyzed to determining their antigen binding affinity. Structural information of the exemplary anti-TACI antibodies is provided in Table 2 above.

### (a) TACI-Binding Activity Via FACS

Anti-TACI scFv binders have been identified through Anti-TACI supernatant FACS screening assay with JVM2, MM1.R and RPMI8226 cell lines as described in above supernatant FACS screening assay. 200 nM of purified scFvs were subjected into FACS binding assay with TACI negative cell lines K562, H929 and TACI expressing cancer cell lines of JVM2, MM1.R, RPMI8226. The assay procedure and data analysis were described in Example 1 above.

Anti-TACI scFvs showed selective binding to target cell lines. The binding level is correlated with TACI receptor number on the cell lines (FIG. 3). The EC₅₀ values of exemplary anti-TACI antibodies (in scFv format) were summarized in Table 10 below.

TABLE 10
EC50 Values of Exemplary Anti-TACI
Antibodies to Various Cell Lines

| | EC₅₀ (nM) | | | | |
|---|---|---|---|---|---|
| Antibodies | JVM2 | MM1R | MM1S | RPMI8226 | H929 |
| TC-01 | 4.3 | 8.673 | 13.39 | 1.068 | ND |
| TC-02 | 40.04 | 10.44 | 25.17 | 22.1 | ND |
| TC-03 | 26.83 | 13.96 | 46.66 | 829.5 | ND |

### (b) TACI-Binding Activity Via ELISA

Purified anti-TACI scFvs were subjected to ELISA binding assay. Recombinant TACI and negative protein were immobilized on the 384 well plate. 200 nM of anti-TACI scFv clones were serial diluted and tested for binding in the assay as described above. The exemplary anti-TACI scFvs generated from live cell selections (see above disclosures) did not bind to soluble recombinant TACI from multiple sources.

### Example 3. Construction of Anti-Bcma Monospecific Chimeric Antigen Receptor (CAR)

This example illustrates construction and characterization of exemplary anti-BCMA monospecific chimeric antigen receptors (CARs).

### Anti-BCMA CAR Construct

Three selected anti-BCMA scFv candidates, BC-05, BC-06, and BC-08, and, were used to construct anti-BCMA monospecific CARs. These exemplary anti-BCMA monospecific CAR constructs contain, from N-terminus to C-terminus, the anti-BCMA scFv, flag tag, IgG4 hinge, spacer, CD28 transmembrane, a 4-1BB co-stimulatory signaling domain, and a CD3z intracellular signaling domain. The amino acid sequences of these exemplary anti-BCMA monospecific CARs are provided in Table 4 above.

### Production of Lentivirus Vectors for Producing Anti-BCMA CARs

Coding sequences for the exemplary anti-BCMA CARs described above were cloned into lenti virus vectors following standard molecular biology methods. The resultant lentiviral vectors were co-transfected with LV-MAX packaging mix using polyethylenimine (PEI) transfection reagents to Expi HEK293 following manufacture's protocol. Transfected cells were grown for 72 hrs at 37 C shaking with 8% CO2 level. Supernatant were harvested by centrifugation at 3200 rpm at RT for 10 mins and vacuum filtration using 0.45 um PES membrane. Virus was concentrated by ultracentrifugation (Beckman Coultier) at 8000 rpm for 1 hr at 4 C. The pellet was then resuspended in Lentivirus stabilizer, aliquoted immediately and stored at −80 C.

### Transduction of BCMA CAR-Encoding Viral Vectors to Immune Cells and Expansion Thereof

PBMCs were isolated from fresh healthy donor's LRS chamber using density gradient centrifugation lymphoprep and SepMate 50 kit from Stemcell Technology. CD3+ Pan T cells were then isolated from PBMCs using EasySep human T cell isolation kit following Stemcell technology protocols. Pan T cells were activated with human T-activator CD3/CD28 dynabeads at 1:1 bead to cell ratio 24 hours and then transduced with lentivirus in the presence of dynabeads and 1 mg/mL protamine sulfate. Spinoculation was done at 300 g for 2 hours at 25 C. Cells and viruses were incubated for 24 hours at 37 C. Next day, cells were removed from beads and viruses. Cells were grown for 2 weeks in 5% human serum containing recombinant human IL15 and IL7 (Peprotech) in X-vivo 15 (Lonza) media. Media were changed every 2-3 days with added fresh cytokines.

### Characterization of CAR-T Cells Expressing Anti-BCMA CAR

### (i) Anti-BCMA CAR Expression

CAR surface expression was assessed by surface staining of Anti-flag tag antibody directly conjugated with Mix-n-stain AF647. Briefly, 100,000 lentivirus transduced T cells were incubated with 0.1 ul of anti-flag-AF647 for 1 hour in dark at 4 C shaking. Cells were spun down at 1,300 rpm for 5 minutes, supernatant removed and washed with 200 uL 1×PBS. The resultant samples were reconstituted in 200 μL of 1×PBS. The percentage surface expression was quantified by reading fluorescence-stained cells on Attune NxT Flow Cytometer. Results from this study show that the CAR-expression levels of the different CAR constructs (see Table 4 above) ranged from 50-85%.

### (ii) Cytotoxic T Lymphocyte (CTL) Activity

Human PBMCs and Pan T cell isolation, virus transduction and T cell expansion were described above. To screen different CAR activity, real time image-based CTL activity assay was performed with target cells engineered with GFP. Briefly, 10,000 of transduced T cells were incubated with 10,000 K562-GFP, BCMA/TACI/K562-GFP respectively and at effector (CAR-T) to target cell ratio of 10:1, 5:1 and 1:1 in RPMI1640 media with 10% FBS. No cytokines were added. The assay was run for 64 hours and GFP of target cells was imaged and quantified by Cytation 5 scanner. IFNγ was detected with Human IFNγ Duoset ELISA kit (R&D System) post CTL assay. Briefly, supernatant was collected after CTL assay terminated at 64 hour. Recombinant IFNγ was serial diluted and included in the assay to create standard curve. Supernatant IFNγ and recombinant IFNγ were assayed following the manufacture's protocol provided. The data was analyzed using Prism 8.0 software.

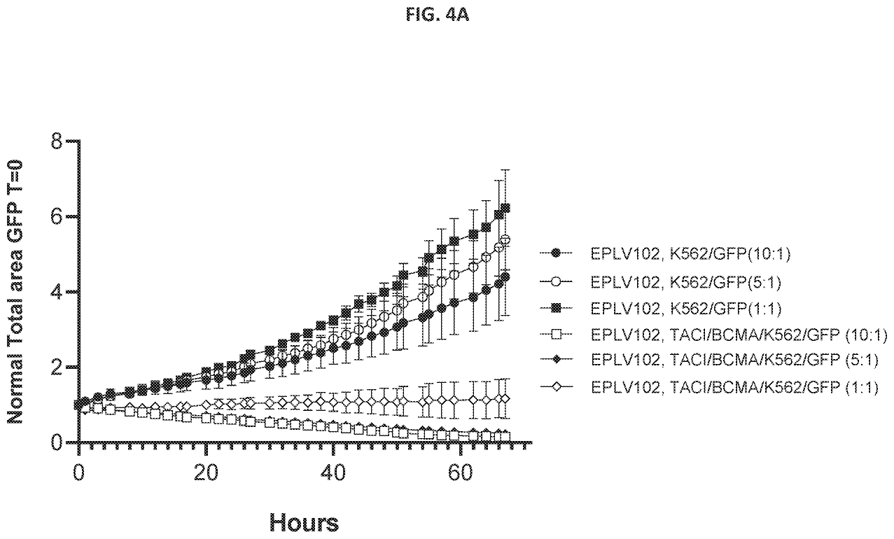

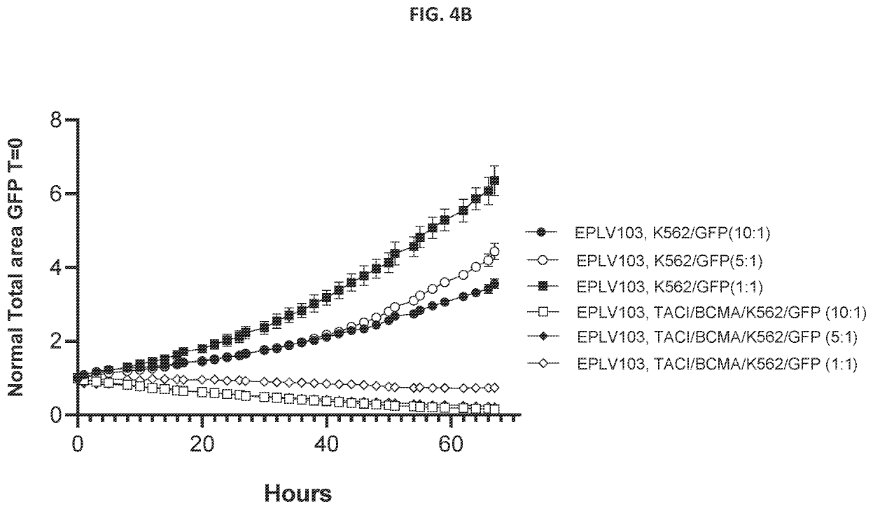

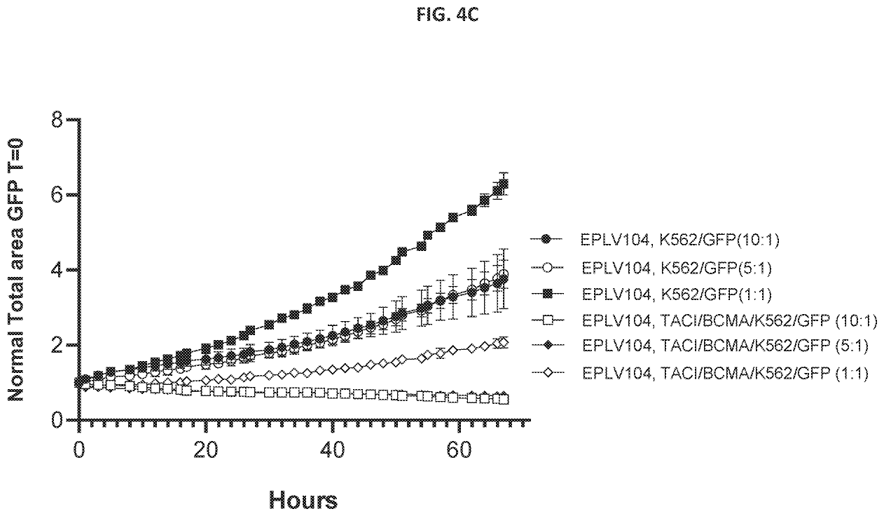

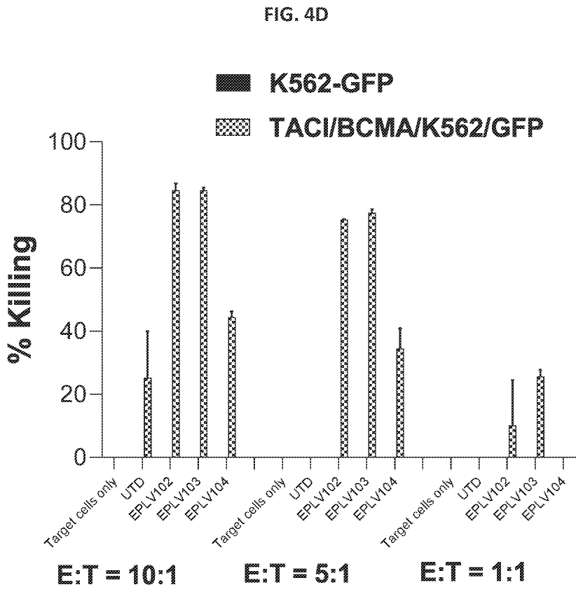

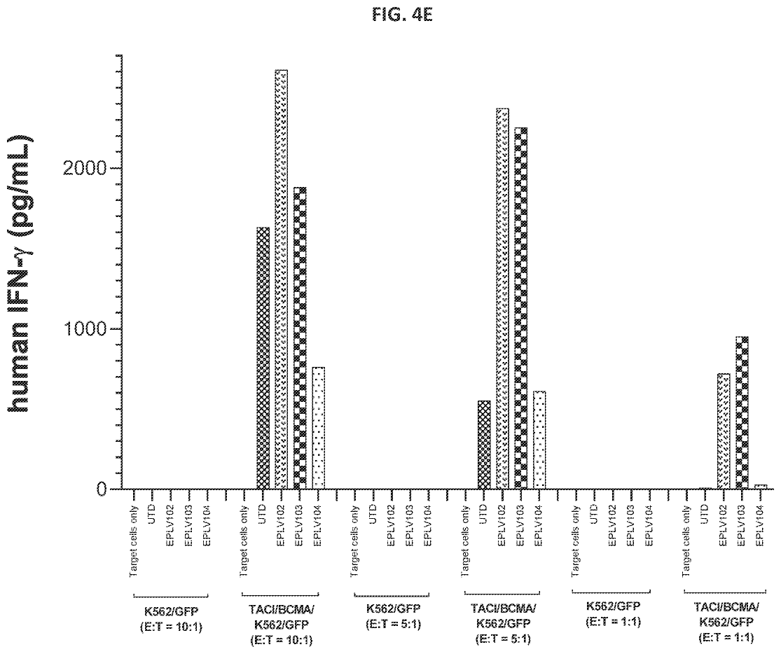

The CTL activity with different target cells and range of effector to target cell ratio was shown in FIGS. 4A-4C. The end point CTL activity of different CARs were calculated as shown in FIG. 4D. The IFNγ level was shown in FIG. 4E. The BCMA mono-specific CARs demonstrated dose dependent target specific CTL activity and correlated with IFNγ secretion. Multiple donors have been screened with different CARs and showed similar CAR CTL activity (FIGS. 5A-5B).

(iii) CTL Assay of CAR-T Cells Expressing Anti-BCMA Construct EPLV102

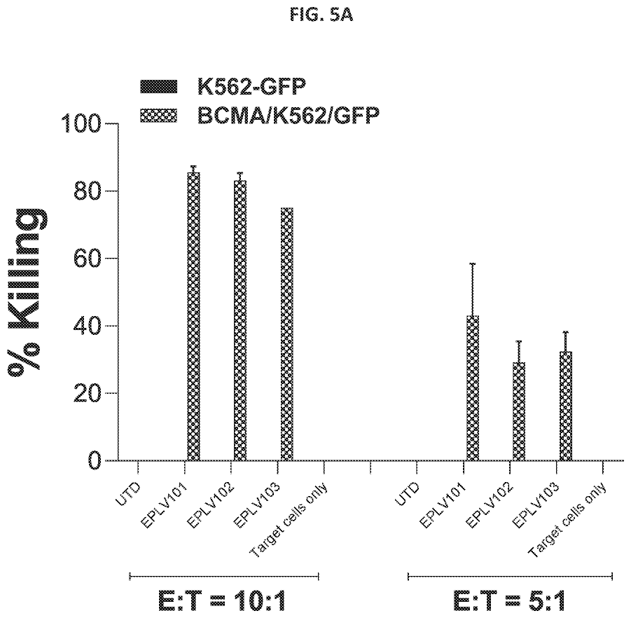

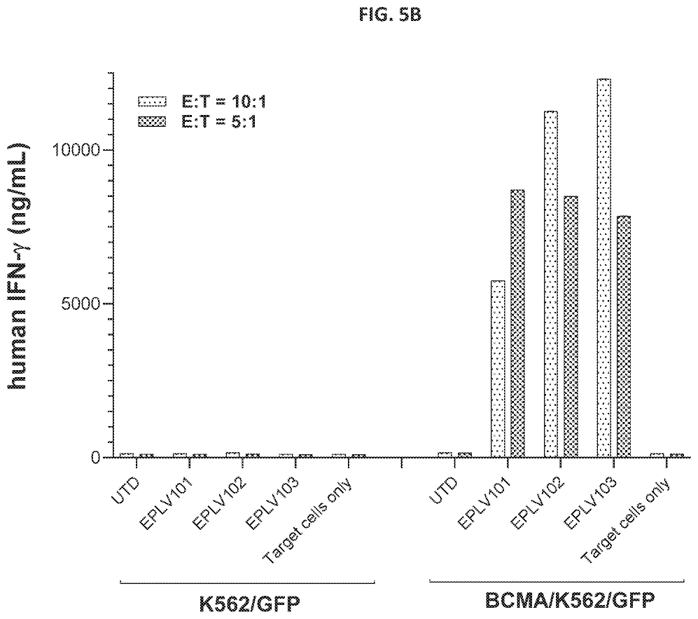

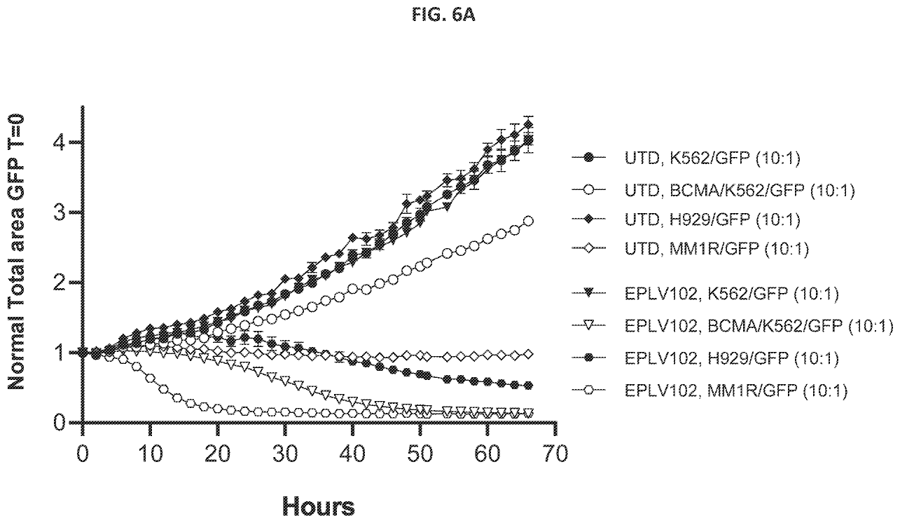

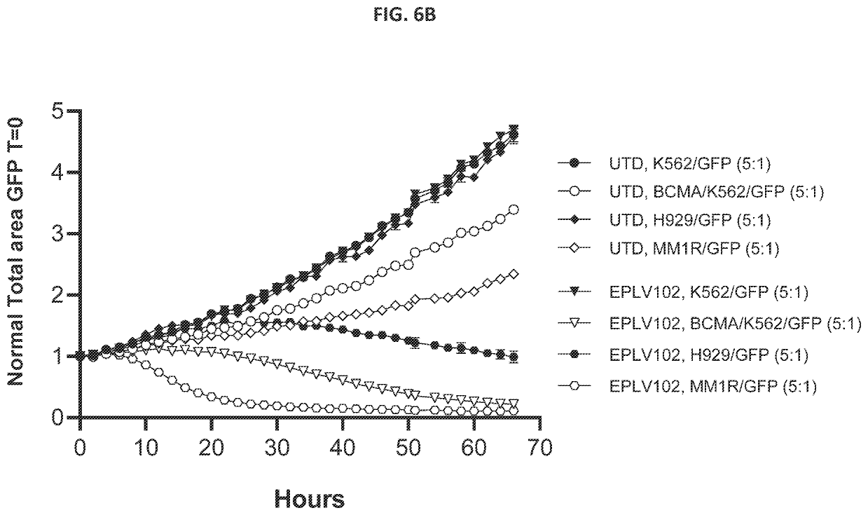

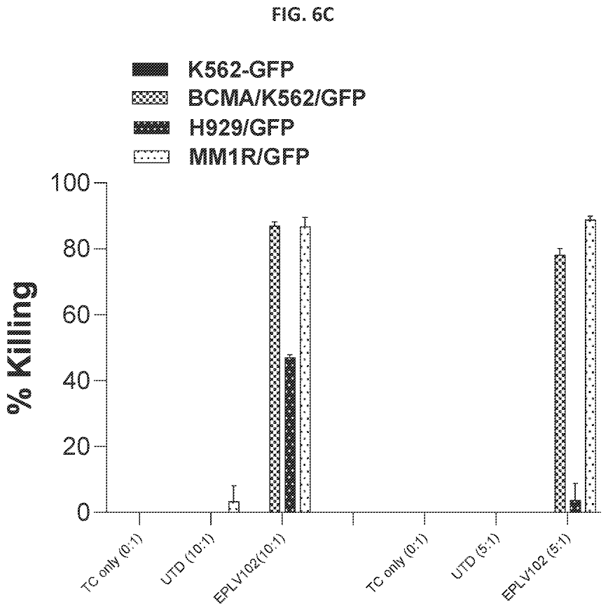

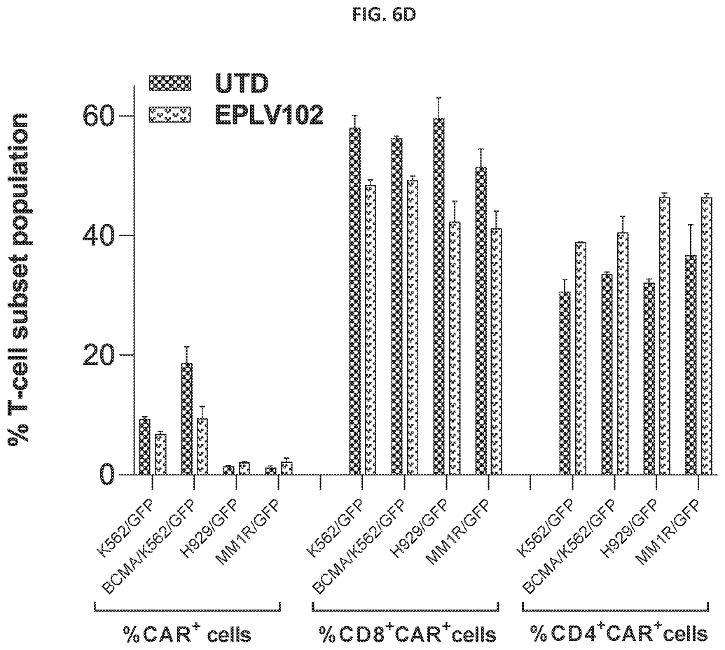

To further evaluate the EPLV102-expressing CAR-T cell CTL activity, PBMCs from multiple donors were transduced with lentivirus and CAR-T expanded as described above. In donor shown here, the transduced or non-transduced T cells were co-cultured with K562, BCMA/K562, H929 and MM1R GFP cells at effector to target cell ratio of 10:1 and 5:1 for 66 hours. The target specific CTL activity was observed in both E/T ratio (FIGS. 6A-6C). Similar CTL activity was observed with multiple donors.

CAR-T cell phenotype is associated with T cell persistency. Following the CTL assay, CAR-T cell phenotype was analyzed using FACS assay with a panel of antibodies detecting T cell differentiation markers. Briefly, anti-CD3, anti-CD4, anti-CD8, anti-CD45RO, anti-CD62L, were used to stain the transduced T cells as described above. Analysis was done by Attune NxT software. The CD3, CD4, CD8 positive CAR-T cells and Tn, Tscm, Tcm and Tem cells were gated and the results showed comparable CD8 and CD4 CAR+ T cells (FIG. 6D) and a high percentage of Tcm (FIG. 6E).

### (iv) EPLV102 CAR-T Persistency Assessed by Multiple Rounds of Target Cell Challenging

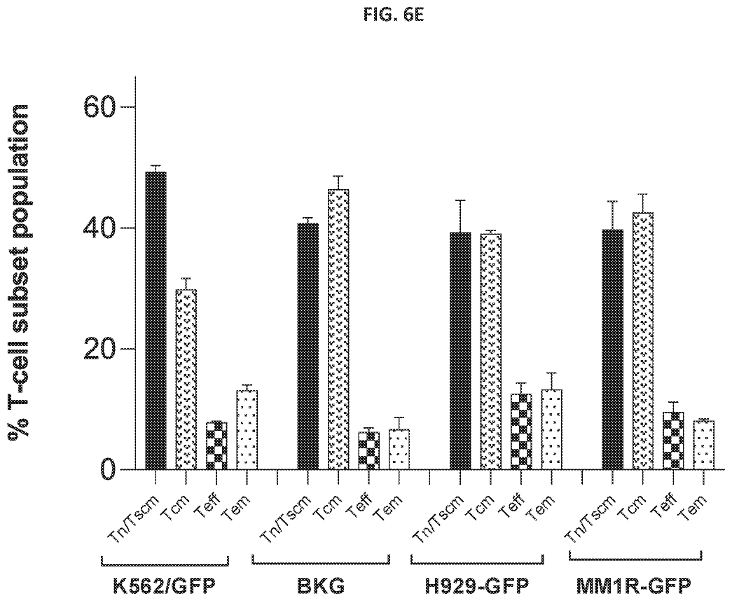

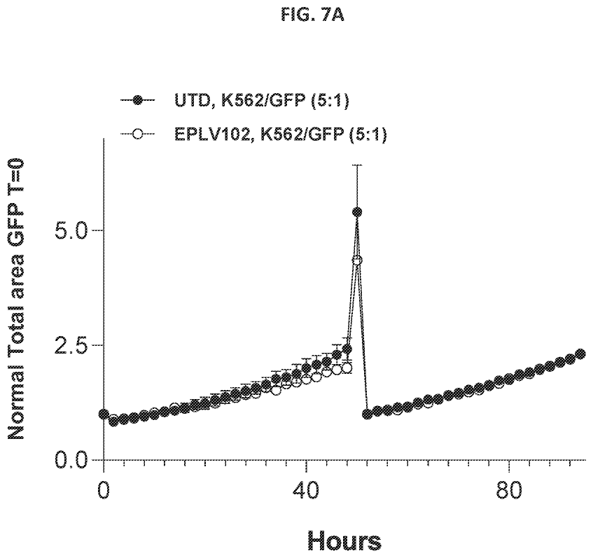

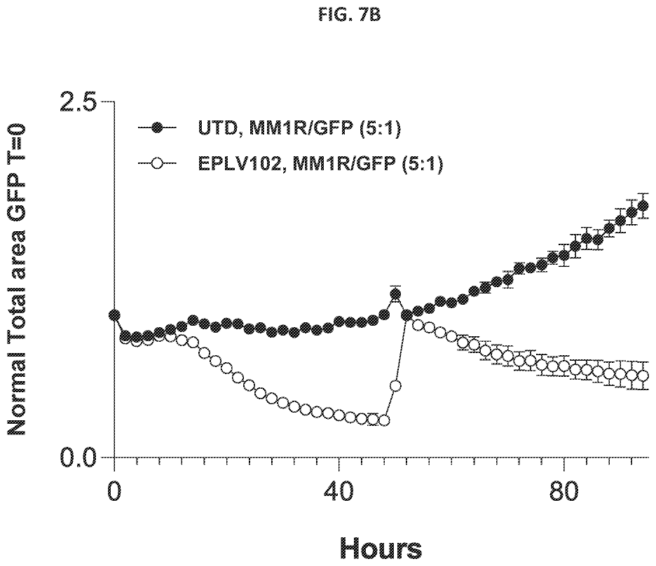

To further test the CAR-T persistence, a multiple rounds of target cell challenging experiment was executed. EPLV102 was transduced into Pan T cells and expanded. 10,000 of transduced T cells were challenged with 10,000 of K562 and MM1R GFP cells for 48 hours, followed by rechallenging the transduced T cells with fresh 10,000 target cells for another 48 hours in RPMI1640 medium without additional cytokines. The CTL activity was quantified imaged every 2 hours with Cytation 5 and analyzed by Prism 8.0 software. EPLV102 demonstrated persistent CTL activity in 2 rounds of target challenge and rechallenge experiment (FIGS. 7A-7B).

### Example 4. Construction of Anti-TACI Monospecific Chimeric Antigen Receptor (CAR)

This example illustrates construction and characterization of exemplary anti-TACI monospecific chimeric antigen receptors (CARs).

### Anti-TACI CAR Constructs and Production of CAR-T Cells Expressing Such

Four selected Anti-TACI scFv candidates, TC-01, TC02, TC-03, and TC04, were used to construct anti-TACI monospecific CARs. These CAR constructs contain, from N terminus to C-terminus, an anti-TACT scFv, flag tag, CD8a spacer and transmembrane, 41BB and CD3z intracellular signaling domains. The amino acid sequences of these anti-TACI CAR constructs are provided in Table 4 above.

Coding sequences for the anti-TACI CAR constructs were cloned int lentiviral vectors following standard molecular biology methods. The resultant lentiviral vectors were used to transduce PBMCs for anti-TACI CAR expression, following conventional practice. See disclosures above.

### Characterization of CAR-T Cells Expressing Anti-TACI CAR

### (i) CTL Activity of Anti-TACI CAR T Cells

Human PBMCs and Pan T cell isolation, virus transduction and T cell expansion were described above. To screen different CAR activity, real time image-based CTL activity assay was performed with target cells engineered with GFP. CAR constructs EPLV200, EPLV254, EPLV255, EPLV256 transduced pan T cells were incubated with 100,000 of K562, H929 TACI negative and MM1R TACI positive GFP cells at effector to target cell ratio of 5:1 and 1:1 for 72 hours as described and imaged by Cytation 5.

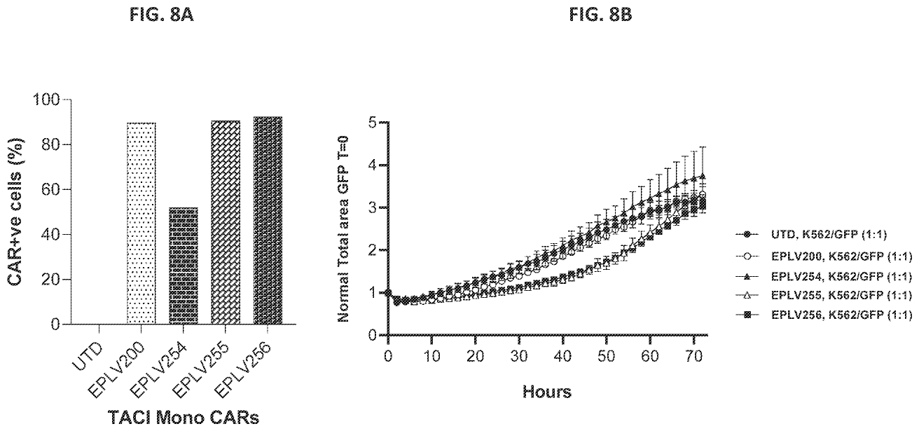

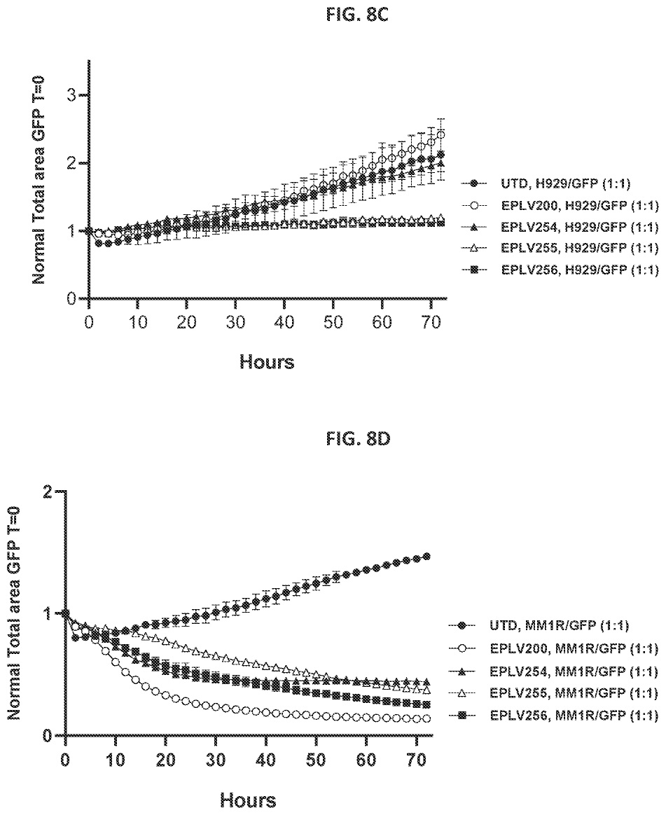

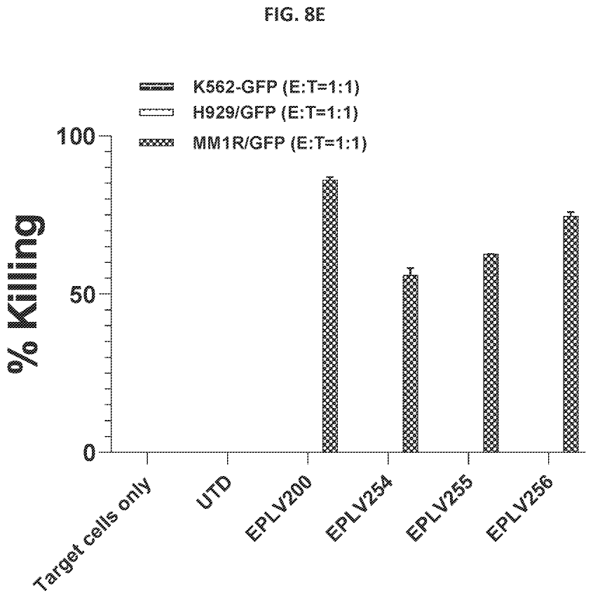

The TACI mono-specific CARs showed 50-90% surface expression (FIG. 8A). The real time CTL activities with different target cells at E/T ratio 1:1 was shown in FIGS. 8B-8D. The end point CTL activity of different CARs were calculated as shown in FIG. 8E. Multiple donors have been screened with different TACI CARs and showed similar results.

### (ii) CAR-T Persistency Assessment

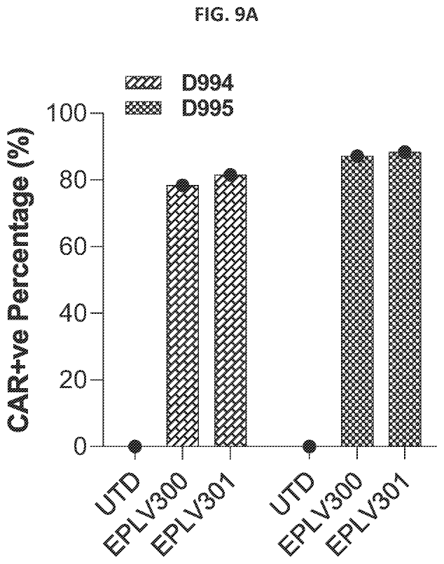

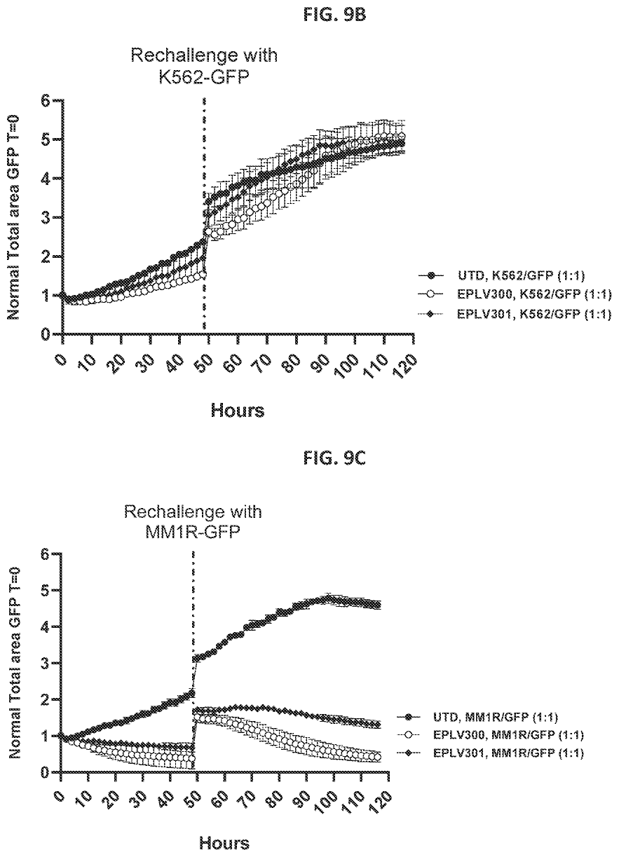

To further test impact of different spacers on CAR-T persistence, a multiple rounds of target cell rechallenge experiment was performed on CAR-T cells expressing EPLV300 (short spacer) and EPLV301 (medium spacer containing IgG4 hinge and CD28 transmembrane). See structure information provided in Table 4 above. Nucleic acids encoding the CAR constructs were transduced into Pan T cells and expanded in 2 donors. 10,000 of transduced T cells were challenged with 10,000 of K562 and MM1R GFP cells for 48 hours, followed by rechallenging the transduced T cells with fresh 20,000 target cells for another 68 hours in RPMI medium without additional cytokines. Both short and medium spacer CAR surface express at 70-90% (FIG. 9A). The CTL activity was quantified by imaging target-GFP cells every 2 hours with Cytation 5 and analyzed by Prism 8.0 software. EPLV300 demonstrated persistent better CTL activity in 2 rounds of target challenge and rechallenge experiment in two donors (FIGS. 9B-9C).

### Example 5: Construction and Characterization of Anti-BCMA and Anti-TACI Bispecific Car

This example illustrates construction and characterization of exemplary anti-BCMA/anti-TACI bi-specific (CARs).

### Anti-BCMA/Anti-TACI Bispecific CAR Constructs

The same anti-BCMA scFvs and anti-TACI scFvs used in the monospecific CAR constructs disclosed above were used to construct anti-BCMA/anti-TACI bi-specific CR constructs, in which the anti-BCMA and anti-TACI scFv fragments are in tandem format in the orientation of anti-BCMA scFv to anti-TACI scFv. The bi-specific CAR constructs further comprise a flag tag, CD8a spacer and transmembrane (EPLV217), or IgG4 hinge, CD28 transmembrane (EPLV302), 4-1BB co-stimulatory signaling domain, and CD3z intracellular signaling domains. The amino acid sequences of the exemplary bi-specific CAR constructs are provided in Table 4 above.

Coding sequences for the bi-specific CAR constructs were cloned int lentiviral vectors following standard molecular biology methods. The resultant lentiviral vectors were used to transduce PBMCs for bi-specific CAR expression, following conventional practice. See disclosures above.

### Characterization of CAR-T Cells Expressing Anti-BCMA/Anti-TACI Bispecific CAR

Human PBMCs and Pan T cell isolation, virus transduction and T cell expansion were described above. The real time image-based CTL activity assay was performed with target cells engineered with GFP. CAR constructs EPLV217 with CD8a TM and EPLV302 with CD28 TM transduced pan T cells were incubated with 100,000 of K562 BCMA⁻/TACI⁻ double negative, H929 BCMA⁺/TACI⁻ and MM1R BCMA⁺/TACI⁺ double positive GFP cells at effector to target cell ratio of 1:1 for 72 hours as described and imaged by Cytation 5 in multiple donors. Target cell rechallenging assay also perform with EPLV302 to check persistence of the bispecific CAR.

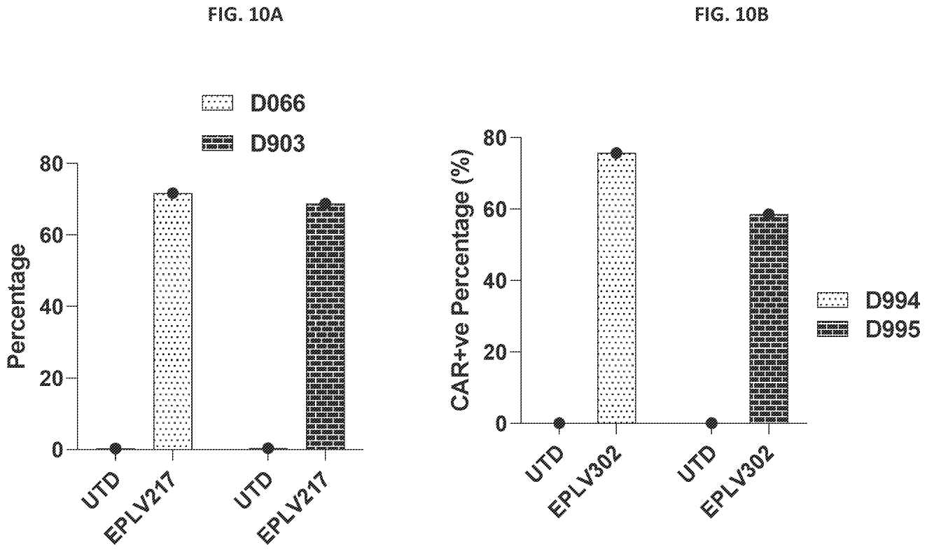

The BCMA/TACI bispecific CARs showed 60-80% surface expression in multiple donors as shown in FIGS. 10A-10B, respectively.

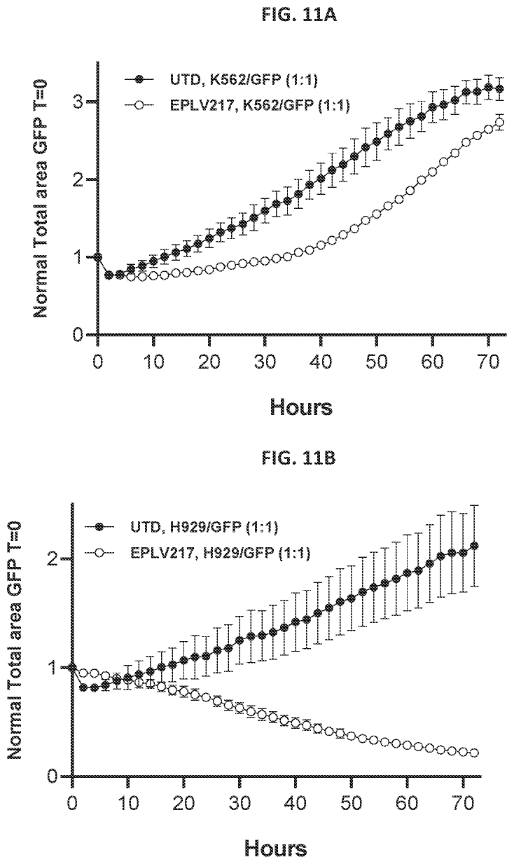

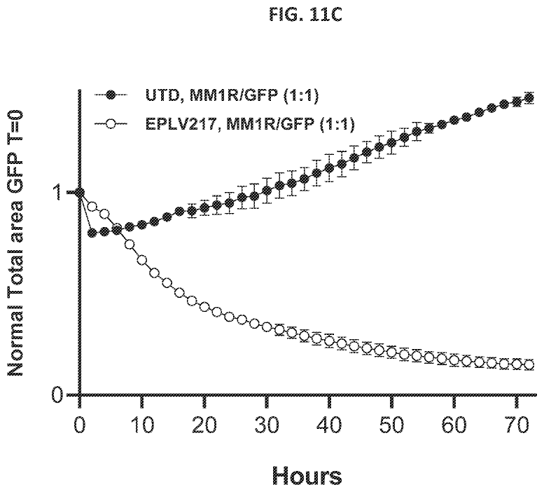

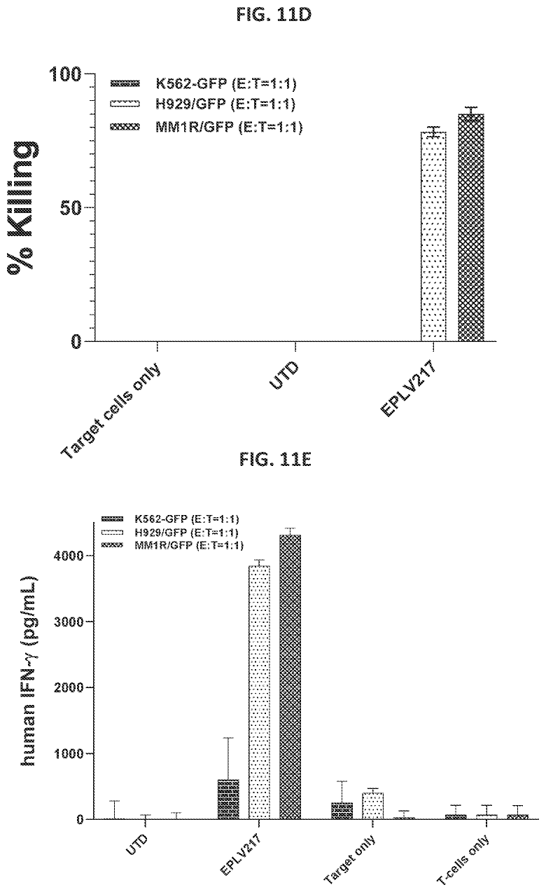

The CTL activity of CAR-T cells expressing EPLV217 against different target cells was shown in FIGS. 11A-11C. The EPLV217-expressing CAR-T cells demonstrated specific and potent CTL activity against different target cell lines (FIG. 11D) and robust IFNγ release (FIG. 11E).

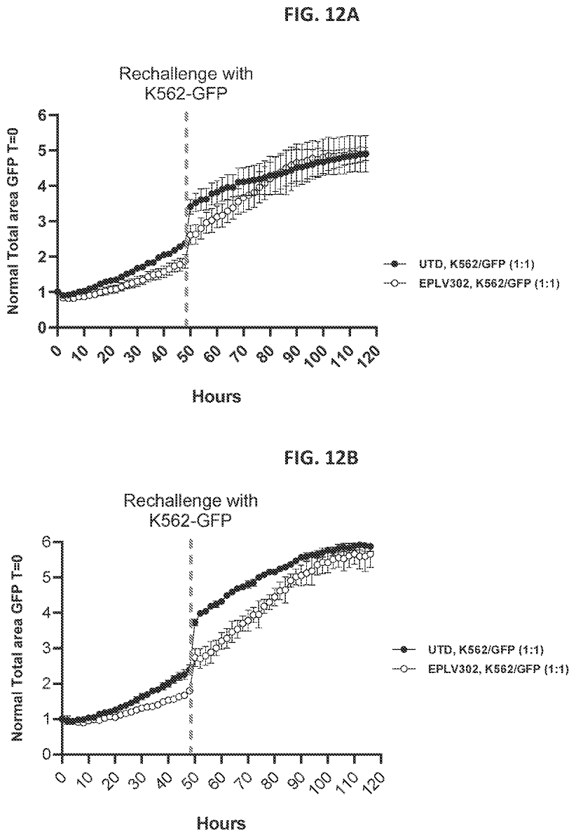

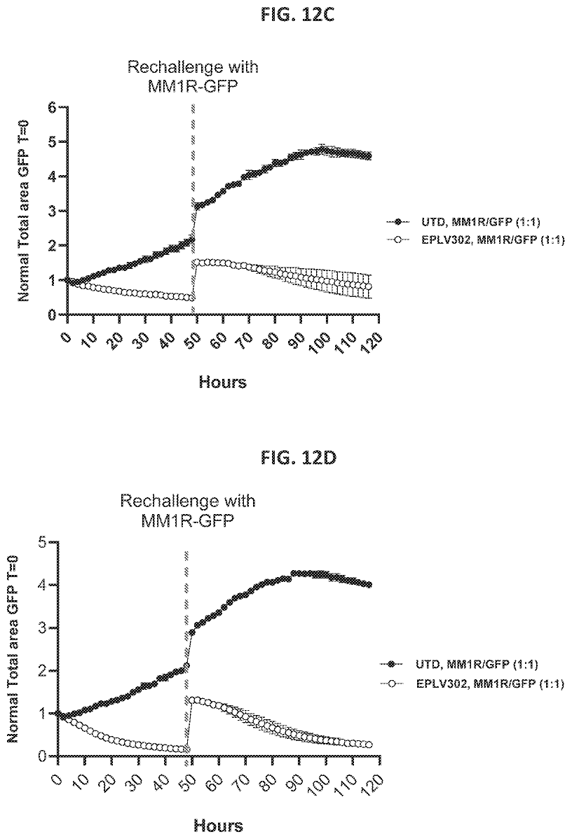

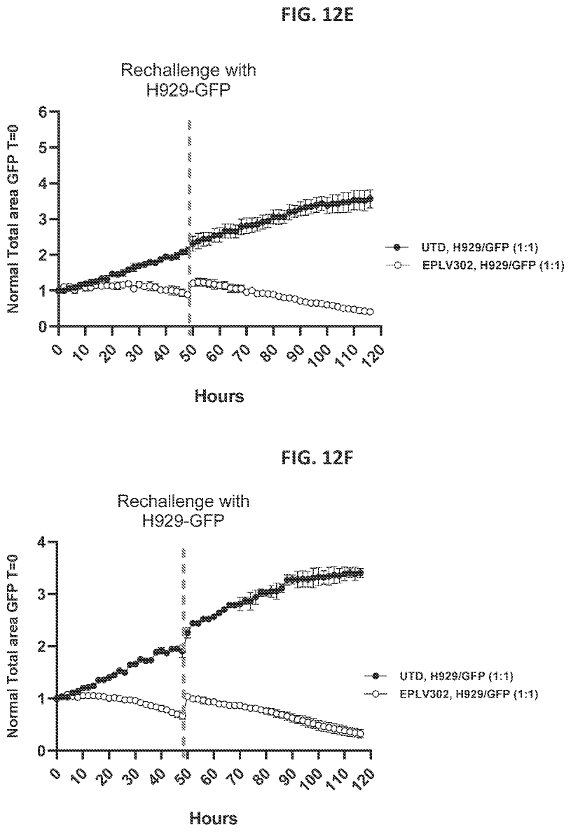

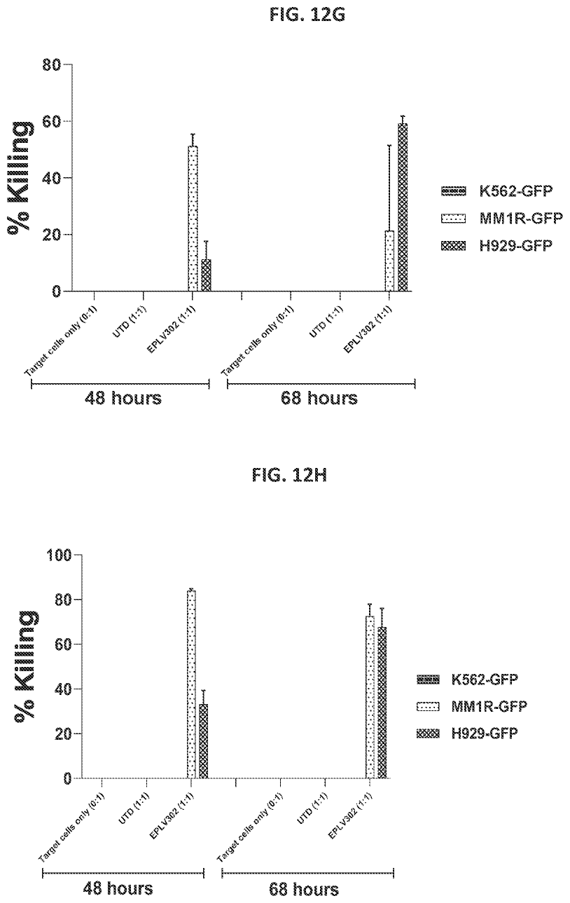

The CTL activity of CAR-T cells expressing EPLV302 also demonstrated potent and persistent target specific CTL activity to target cell lines in rechallenging assay (FIGS. 12A-12H). The IFNγ level correlated with CTL activity in different target cells (FIG. 12I). Multiple donors have been screened with both format of CARs and showed similar results.

### Example 6: In Vivo Efficacy Study in Disseminated MM1R-Luciferase Model

In vivo anti-tumor efficacy of anti-BCMA mono-specific, anti-TACI mono-specific and anti-BCMA/anti-TACI bispecific CAR-T cells were evaluated in a disseminated MM1R-luciferase model in NCG mouse. Three CAR constructs, EPLV200, EPLV257, and EPLV217, were transduced in Pan T cells and expand in vitro for 4 days. 5×10⁵ of MM1R-luciferase cells were inoculated to NCG mice. At day 8, PBS control and 1e6 of each CAR-T cells were dosed to the mice. The mice were imaged every 3-4 days and body weight were measured.

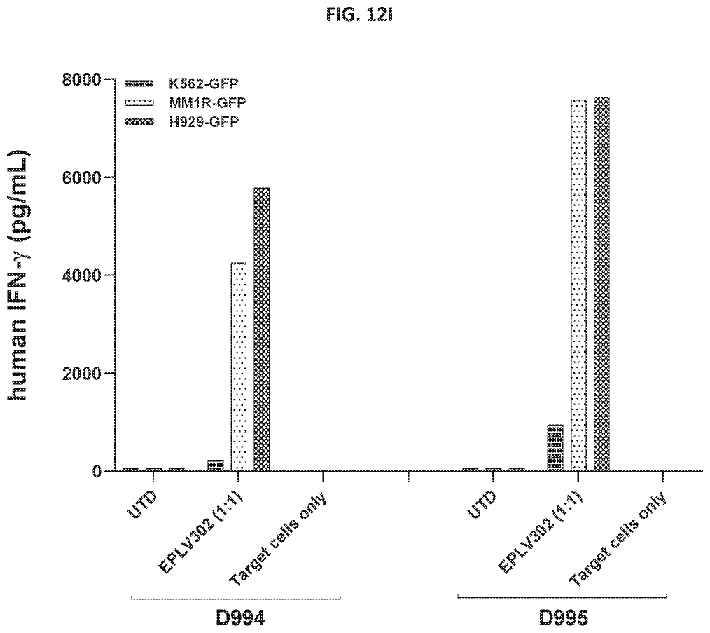

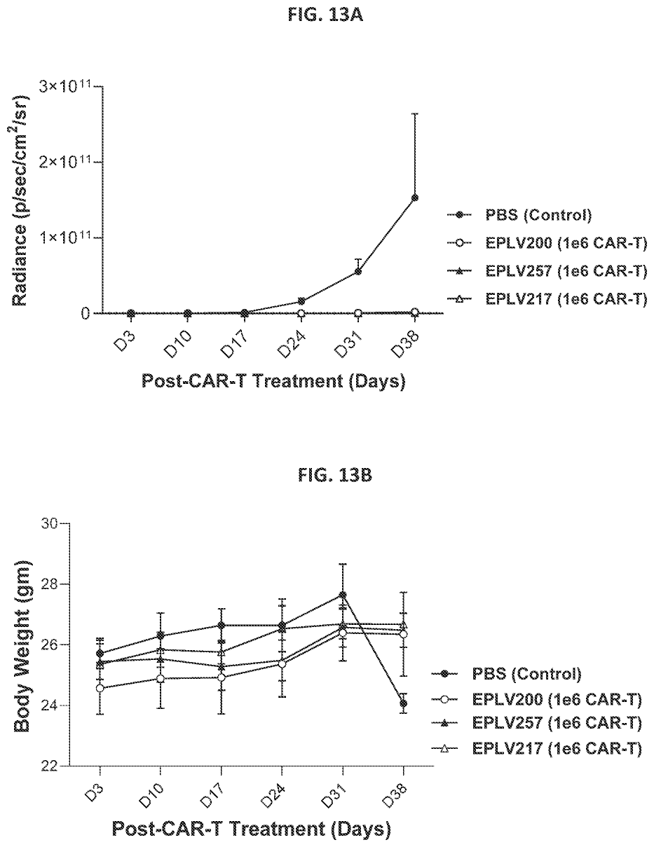

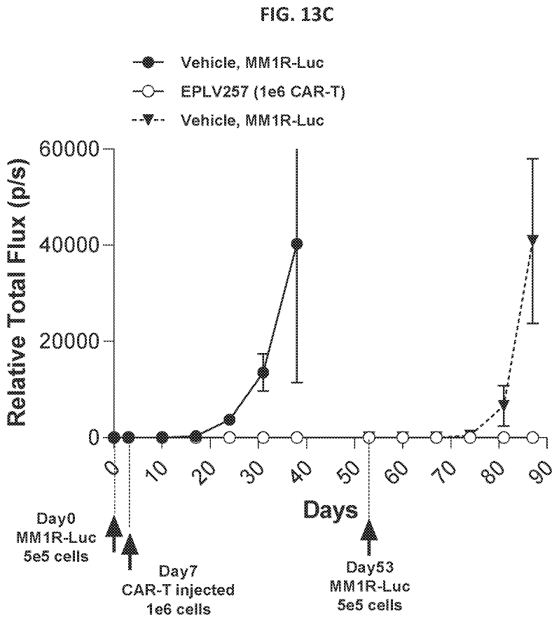

As indicated in FIG. 13A, all treatment groups showed tumor growth inhibition and all CAR-Ts efficiently eradicated tumor cells in all mice at day 38. There was no body weight loss in treatment groups (FIG. 13B). All mice survived during this study.

The BCMA mono-specific CAR-T treated group was also rechallenged with 5×10⁵ of MM1R-luciferase cells at day 53. No additional CAR-T cells injected. No tumor growth detected in BCMA CAR-T (EPLV257) treated mice up for to 87 days. All mice survived. The control mice treated with PBS showed robust tumor growth (FIG. 13C)

### Example 7: Characterization of Engineered T Cells Expressing Anti-BCMA/TACI Bispecific Car and Armor Polypeptide

This example evaluates bioactivities of engineered T cells expressing an anti-BCMA/TACI bispecific CAR construct and an armor polypeptide comprising an anti-PDL1 fragment and a mutant IL2 fragment.

An anti-BCMA/TACI bispecific CAR (EPLV302) having the following components (from N-terminus to C-terminus) were constructed: signal peptide, anti-BCMA scFv, peptide linker, anti-TACI scFv, flag tag, CD8a spacer and transmembrane (or IgG4 hinge), CD28 transmembrane, 41BB co-stimulatory domain, and CD3z intracellular signaling domains. To co-express the armor polypeptide, the coding sequence for the anti-BCMA/TACI bispecific CAR was in connection (at its 3′ end) with coding sequences for, from 5′ to 3′, a T2A peptide, a second signal peptide, anti-PDL1 fragment and a mutantIL-2. See EPLV302 (without armor polypeptide) and EPLV327 (with the armor polypeptide) structure information provided in Table 4 above. The coding sequences were cloned into a Lentivirus vector following standard molecular biology methods.

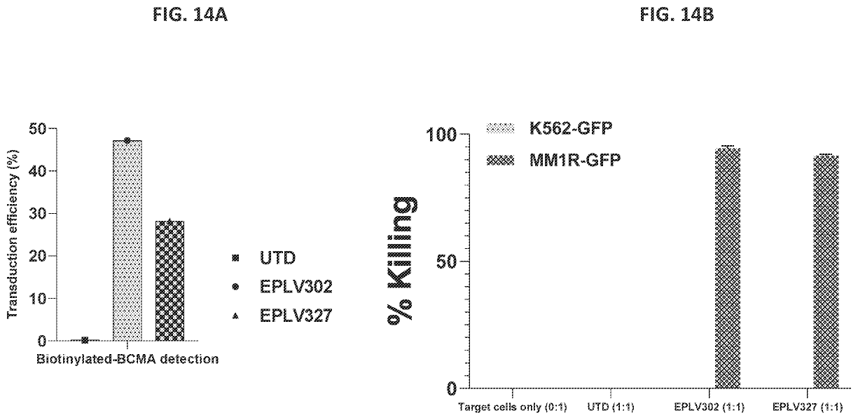

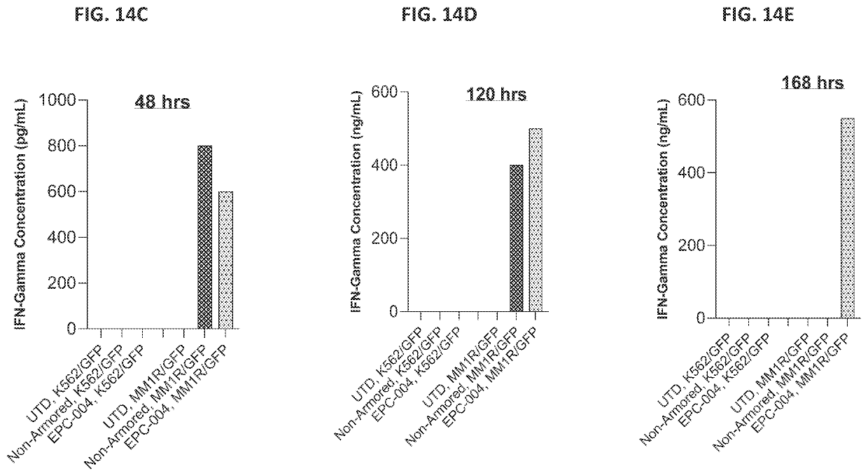

Human PBMCs and Pan T cell were isolated and transduced by the viral vectors disclosed above, and expanded as disclosed in the above Examples. Expression of the bispecific CAR in T cells transduced by either the bispecific CAR coding sequence, or in combination with the coding sequence for the armor polypeptide was examined. The engineered T cells showed around 25-50% surface expression of the bispecific CAR, with or without the co-expressed armor polypeptide. FIG. 14A.

Further, a real time image-based CTL activity assay was performed with target cells engineered to express GFP. Briefly, pan T cells engineered with CAR construct EPLV302 (non-armored) or construct EPLV327 (armored) were incubated with 100,000 of K562 BCMA−/TACI− double negative or MM1R BCMA+/TACI+ double positive GFP cells at an effector-to-target cell ratio of 1:1 for 114 hours as described and then imaged by Cytation 5 in multiple donors. As shown in FIG. 14B, T cells expressing the bispecific CAR, with or without the armor polypeptide, showed high cell killing activity against the MM1R-GFP cells, but not the K562-GFP cells.

Nest, a target cell rechallenging assay was perform using pan T cells engineered either EPLV302 or EPLV327 at an effector-to-target cell ratio of 0.5:1 for 168 hours to investigate persistence of the T cells expressing the bispecific non-armored and armored CAR. Both non-armored and armored bispecific CAR-T cells demonstrated persistent CTL activity in up to 168 hours in cancer cell rechallenging assay. Further, Release of IFNγ was measured at 48 hours, 120 hours, and 168 hours. As shown in FIGS. 14C-14E, the IFNγ levels measured at 48-hr and 120-hr correlated with the CTL activity using different target cells and the IFNγ level of T cells expressing armored CAR at 168-hr was higher than that of T cells expressing the non-armored CAR. FIG. 14E. Multiple donors have been screened with both format of CARs and showed similar results.

### Example 8: In Vivo Anti-Tumor Activity of Engineered T Cells Expressing Anti-BCMA/TACI Bispecific Car and Armor Polypeptide

To evaluate the anti-tumor activity of engineered T cells expressing the bispecific armored CAR and the armor polypeptide (EPLV327; see Table 4 above), 6-8-week-old female NCG mice (Charles River Laboratories, Wilmington, MA) were inoculated intravenously with 5×10⁵ MM1R-luc multiple myeloma tumor cells suspended in serum free media. Seven days after tumor cell inoculation, mice were randomized into 3 treatment groups as follows:
(1) vehicle (n=5 mice),
(2) non-armored CAR (1E6 CAR-T cell dose; n=18 mice) and
(3) EPC-004 (EPLV327) (1E6 CAR-T cell dose; n=18 mice).

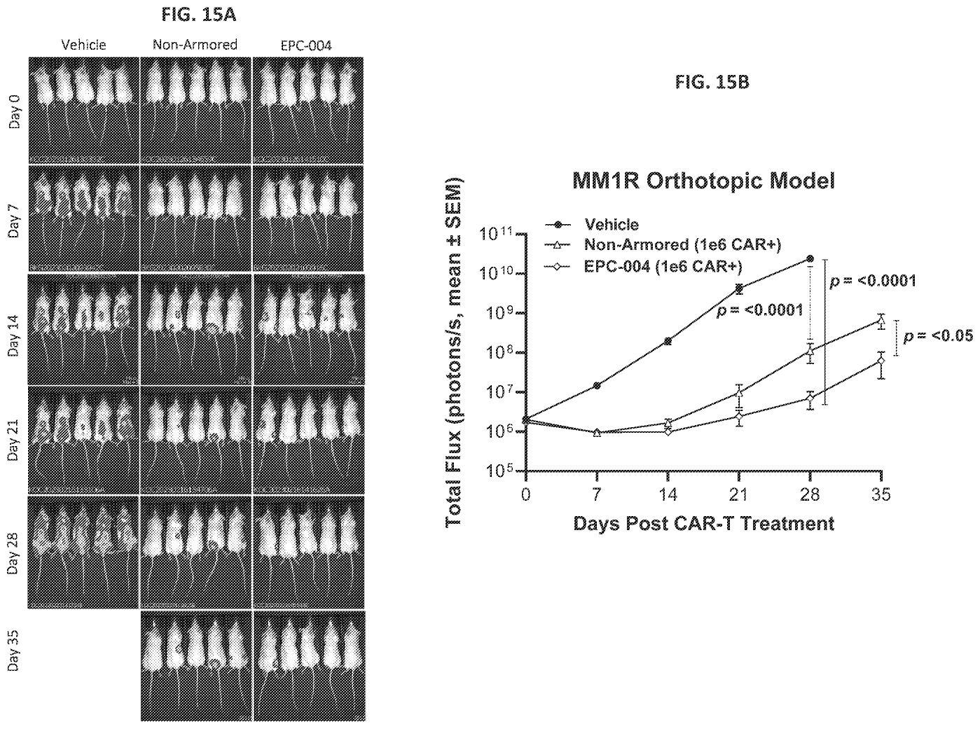

CAR-T cells were administered via intravenous injection seven days after tumor cell inoculation. Tumor burden was determined once per week by bioluminescence imaging after intra-peritoneal injection of luciferin substrate. Mice of the vehicle group showed high tumor burden, while the tumor burden of the Group (2) and Group (3) mice were significantly reduced. FIG. 15A. Animals treated with non-armored CAR and EPC-004 demonstrated significant tumor growth inhibition over time relative to vehicle control group.

Further, EPC-004 CAR-T cells exhibited significantly greater anti-tumor activity as compared to the non-armored CAR-T cells as shown in FIG. 15B. Animals were monitored over time for adverse clinical events related to tumor burden. By day 28 after treatment start, all vehicle control animals had been euthanized due to tumor burden whereas animals in both non-armored CAR and EPC-004 treatment groups survived past day 35.

### Example 9: Development of CAR-NK Cells

The BCMA mono-specific (EPLV310) and TACI mono-specific (EPLV300) CAR candidates (see Table 4 above), including an anti-BCMA scFv or anti-TACI scFv, flag tag, IgG4 hinge, CD28 transmembrane, 41BB and CD3z intracellular signaling domains, were constructed following routine methods. The bispecific CAR (EPLV302) candidate, in a tandem format, was also constructed. The structural information of this bispecific CAR is also provided in Table 4 above. The coding sequences of the monospecific and bispecific CAR constructs were cloned into a Lentivirus vector following standard molecular biology methods.

The lentivirus carrying coding sequences for the monospecific or bispecific CAR was produced as described before. PBMCs were isolated from fresh healthy donor's LRS chamber using density gradient centrifugation lymphoprep and SepMate 50 kit from Stemcell Technology. NK cells were then isolated from the PBMCs using Miltenyi's NK cell isolation kit following the manufacture protocols. NK cells were activated with CTS X-pander media (ThermoFisher) and cytokine cocktails for 6-7 days at 0.5-1×106 cells/mL concentration. After activation, 1 mg/mL protamine sulfate and 1 uM BX795 was added. Spinoculation was done at 1000 g for 45 minutes at 32° C. The NK cells and viruses were incubated for 24 hours at 37° C. Next day, viruses were removed and transduced NK cells replenished with fresh media and cytokine cocktail. Media were changed every 2-3 days with added fresh cytokines.

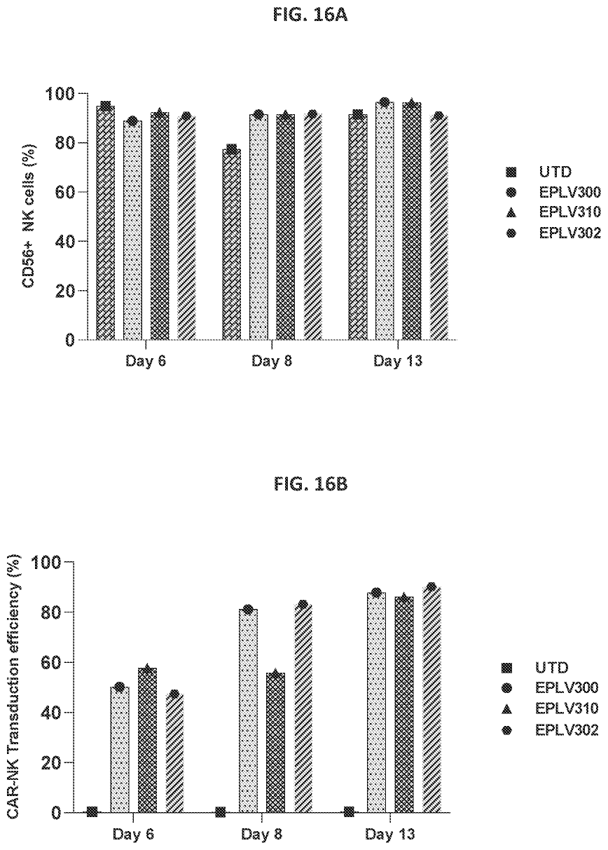

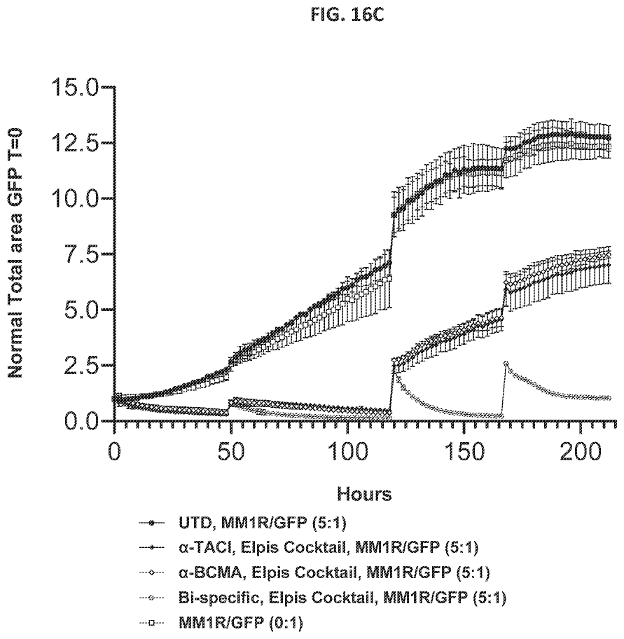

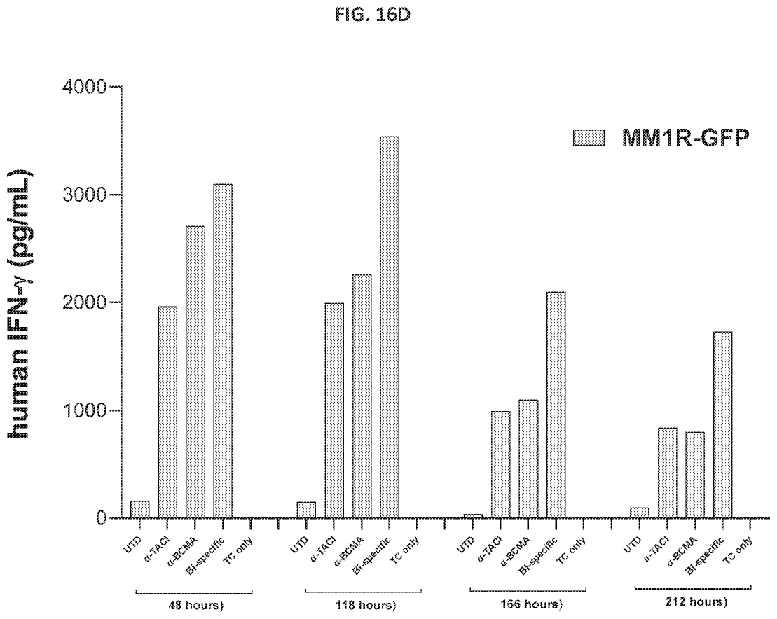

The CAR surface expression in the transduced NK cells was assessed by surface staining using CD56-PE and AF647 conjugated Anti-flag tag antibody respectively. Briefly, 100,000 lentivirus transduced NK cells were incubated with 0.5 ul of CD56-PE and 0.1 ul of anti-flag-AF647 for 1 hour in dark at 4° C. shaking. Cells were spun down at 1,300 rpm for 5 minutes, supernatant removed and washed with 200 uL 1×PBS. The resultant samples were reconstituted in 200 uL of 1×PBS. The percentage surface expression was quantified by reading the fluorescence-stained cells on Attune NxT Flow Cytometer. FIG. 16A shows the levels of NK cells expressing CD56, which is an NK cell marker. Around 90-95% purity of the NK cells was observed during expansion. CAR-expression of different CAR constructs gated on NK cell population ranged from 40-90%. FIG. 16B.

Human PBMCs and NK cell isolation, virus transduction and T cell expansion were described above. Both BCMA, TACI-monospecific and BCMA/TACI bispecific CARs showed robust surface expression in multiple donors as shown above. The real time image-based CTL activity assay was performed with MM1R target cells engineered with GFP. CAR constructs EPLV302 (Bispecific), EPLV310 (BCMA mono-specific) and EPV300 (TACI mono-specific) transduced NK cells were incubated with 100,000 of MM1R BCMA+/TACI+ double positive GFP cells at effector to target cell ratio of 5:1 for 212 hours as described and imaged by Cytation 5 in multiple donors. Target cell rechallenging assay was also performed. All CAR-NK constructs demonstrated cancer cell killing activity. The bispecific CAR-NK demonstrated more persistent activity than mono-specific CAR-NK in 3rd and 4th rechallenging (FIG. 16C). The robust IFNγ level release correlated with the persistent CTL activity (FIG. 16D).

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

1. A bi-specific chimeric antigen receptor (CAR) specific to B-cell mature antigen (BCMA) and transmembrane activator and CAML interactor (TACI), the bi-specific CAR comprising:
(a) a first antigen binding moiety specific to TACI;
(b) a second antigen binding moiety specific to BCMA;
(c) a co-stimulatory signaling domain; and
(d) a cytoplasmic signaling domain.

2. The bi-specific CAR of claim 1, wherein the first antigen binding moiety specific to TACI of (a) comprises a heavy chain variable region (V_{H}) and a light chain variable region (V_{L}); wherein the V_{H} comprise the same heavy chain CDRs as those in a reference antibody, and the V_{L} comprise the same heavy chain CDRs as those in the reference antibody; and wherein the reference antibody is TC-01, TC-02, TC-03, or TC-04; optionally wherein the reference antibody is TC-01.

3. The bi-specific CAR of claim 2, wherein the V_{H} and V_{L} of the first antigen binding moiety specific to TACI are identical to the V_{H} and V_{L} of the reference antibody.

4. The bi-specific CAR of claim 1, wherein the first antigen binding moiety specific to TACI is a single chain variable fragment (anti-TACI scFv).

5. The bi-specific CAR of claim 4, wherein the anti-TACI scFv comprises an amino acid sequence of any one of SEQ ID NOs: 75, 84, 93, and 102; optionally wherein the anti-TACI scFv comprises the amino acid sequence of SEQ ID NO: 75.

6. The bi-specific CAR of claim 1, wherein the second antigen binding moiety specific to BCMA of (a) comprises a heavy chain variable region (V_{H}) and a light chain variable region (V_{L});
wherein the V_{H} comprises:
(hi) a heavy chain complementarity determining region (CDR) 1, which comprises X₁YX₂MH, in which X₁ is S or D, and X₂ is A or G;
(hii) a heavy chain CDR2, which comprises
(hii-a) X₃IX₄YDGSX₅KYYADSVKG (SEQ ID NO: 1), in which X₃ is V or F, X₄ is S or R, and X₅ is D or N;
(hii-b)
(SEQ ID NO: 27)
FIRSKAYGGTTEYAASVKG;
or
(hii-c)
(SEQ ID NO: 43)
GISWNSGSIGYADSVKG;
and
(hiii)
a heavy chain CDR3, which comprises
(hiii-a)
(SEQ ID NO: 56)
DEHQVVPNYRFDF,
(hiii-b)
(SEQ ID NO: 35)
DWEDPLYYYDTPF,
(hiii-c)
(SEQ ID NO: 16)
DWDYYDSSGYYPDALGI,
(hiii-d)
(SEQ ID NO: 9)
DLWDGIVGAPAGY,
(hiii-e)
(SEQ ID NO: 22)
DLTTITPGY,
(hiii-f)
(SEQ ID NO: 63)
DLWEFGGDYADY,
(hiii-g)
(SEQ ID NO: 28)
GPHYDILTSNWFDP,
or
(hiii-h) VQX₆PGAFDI (SEQ ID NO: 181), in which X₆ is P or S; and
wherein the V_{L} comprises:
(li) a light chain CDR1, which comprises
(li-a) SGSGSNIGSNDVS (SEQ ID NO: 58),
(li-b) QASQDIX₇NYLN (SEQ ID NO: 2), in which X₇ is N or S, or
(li-c) RX₈X₉X₁₀ISSYLX₁₁ (SEQ ID NO: 3), in which X₈ is A or S, X₉ is S or T, X₁₀ is G or S, and X11 is G or N;
(lii) a light chain CDR2, which comprises
(lii-a) WNDQRPS (SEQ ID NO: 59),
(lii-b) DASNX₁₂ET (SEQ ID NO: 4), in which X₁₂ is L or V, or
(lii-c) AX₁₃SX₁₄LQS (SEQ ID NO: 5), in which X₁₃ is A or T, and X₁₄ is S or T; and
(liii) a light chain CDR3, which comprises
(liii-a)
(SEQ ID NO: 60)
AAWDDSLNGWV,
(liii-b) QQYDX₁₅LPX₁₆T (SEQ ID NO: 6), in which X₁₅ is K or N, and X₁₆ is F, L, or Y,
(liii-c)
(SEQ ID NO: 32)
QHSYSTPHT,
or
(liii-d)
(SEQ ID NO: 48)
QQLYS.

7. The bi-specific CAR of claim 6, wherein the V_{H} of the second antigen binding moiety specific to BCMA comprise the same heavy chain CDRs as those in a reference antibody, and/or wherein the V_{L} of the second antigen binding moiety specific to BCMA comprise the same heavy chain CDRs as those in the reference antibody; the reference antibody being BC-01, BC-02, BC-03, BC-04, BC-05, BC-06, BC-07, BC-08 or BC-09; optionally wherein the reference antibody is BC-06.

8. The bi-specific CAR of claim 7, wherein the V_{H} and V_{L} of the second antigen binding moiety specific to BCMA are identical to the V_{H} and V_{L} of the reference antibody.

9. The bi-specific CAR of claim 1, wherein the second antigen binding moiety specific to BCMA is a single chain variable fragment (anti-BCMA scFv).

10. The bi-specific CAR of claim 9, wherein the anti-BCMA scFv comprises an amino acid sequence of any one of SEQ ID NOs: 15, 20, 25, 34, 41, 50, 53, 62, and 66; optionally wherein the anti-BCMA scFv comprises the amino acid sequence of SEQ ID NO:50.

11. The bi-specific CAR of claim 1, wherein the co-stimulatory signaling domain is from a co-stimulatory molecule selected from the CD28, 4-1BB, OX40, ICOS, CD27, CD40, or CD40L.

12. The bi-specific CAR of claim 11, wherein the cytoplasmic signaling domain is from CD3ζ.

13. The bi-specific CAR of claim 1, wherein the bi-specific CAR comprises:
(a) a fusion polypeptide comprising, from N-terminus to C-terminus, (i) the first antigen binding moiety, (ii) the second antigen binding moiety, (iii) the co-stimulatory signaling domain, and (iv) the cytoplasmic signaling domain; or
(b) a fusion polypeptide comprising, from N-terminus to C-terminus, (i) the second antigen binding moiety, (ii) the first antigen binding moiety, (iii) the co-stimulatory signaling domain, and (iv) the cytoplasmic signaling domain.

14. The bi-specific CAR of claim 13, further comprising a hinge domain and a transmembrane domain, which are located between (ii) and (iii).

15. The bi-specific CAR of claim 13, further comprising a peptide linker connecting the first antigen binding moiety and the second antigen binding moiety.

16. The bi-specific CAR of claim 15, wherein the peptide linker comprises the amino acid sequence of GGGGS (SEQ ID NO: 104), GGGGSGGGGS (SEQ ID NO: 105), GGGGSGGGGSGGGGS (SEQ ID NO: 106), or GSTSGSGKPGSGEGSTKG (SEQ ID NO: 107).

17. The bi-specific CAR of claim 1, further comprising a signal peptide at the N-terminus.

18. The bi-specific CAR of claim 1, which comprises the amino acid sequence of SEQ ID NO: 182.

19. The bi-specific CAR of claim 18, which comprises the amino acid sequence of SEQ ID NO: 165 or 166.

20. A nucleic acid or a set of nucleic acids, which collectively encode the bi-specific CAR of claim 1.

21. The nucleic acid or set of nucleic acids of claim 20, wherein the nucleic acid comprises a first nucleotide sequence encoding the bi-specific CAR, which comprises:
(a) a fusion polypeptide comprising, from N-terminus to C-terminus, (i) the first antigen binding moiety, (ii) the second antigen binding moiety, (iii) the co-stimulatory signaling domain, and (iv) the cytoplasmic signaling domain; or
(b) a fusion polypeptide comprising, from N-terminus to C-terminus, (i) the second antigen binding moiety, (ii) the first antigen binding moiety, (iii) the co-stimulatory signaling domain, and (iv) the cytoplasmic signaling domain.

22. The nucleic acid or set of nucleic acids of claim 21, wherein the nucleic acid further comprises a second nucleotide sequence encoding an armor polypeptide, which enhances T cell functionality, and a third nucleotide sequence encoding a self-cleaving peptide, which is located between the first and second nucleotide sequences.

23. The nucleic acid or set of nucleic acids of claim 22, wherein the armor polypeptide is IL-2, IL-5, IL-15, a co-stimulatory ligand, an anti-PDL1 antibody, or a fusion polypeptide comprising the anti-PDL1 antibody; optionally wherein the anti-PDL1 antibody is a single chain variable fragment (scFv), which is fused to an IL-2 polypeptide.

24. The nucleic acid or set of nucleic acid of claim 23, wherein the armor polypeptide comprises an amino acid sequence of SEQ ID NO: 168, 170, 172, 174, 178, or 180; optionally wherein the armor polypeptide comprises the amino acid sequence of SEQ ID NO: 178.

25. The nucleic acid or set of nucleic acids of claim 20, wherein the nucleic acid(s) is an expression vector(s), optionally a viral vector(s).

26. A genetically engineered immune cell, which expresses the bi-specific CAR of claim 1, and optionally further expresses an armor polypeptide, which enhances T cell functionality.

27. The genetically engineered immune cell of claim 26, which expresses (a) the bispecific CAR comprising the amino acid sequence of SEQ ID NO: 165 or 166, and (b) the armor polypeptide comprising the amino acid sequence of SEQ ID NO: 177 or 178, or the armor polypeptide comprising the amino acid sequence of SEQ ID NO: 173 or 174.

28. The genetically engineered immune cell of claim 26, which comprises the nucleic acid encoding the bi-specific CAR.

29. The genetically engineered immune cell of claim 26, which is a T cell, an NK cell, or a macrophage, optionally wherein the immune cell is a T cell.

30. An anti-TACI chimeric antigen receptor (CAR), comprising an extracellular antigen binding domain specific TACI, a co-stimulatory signaling domain, and a cytoplasmic signaling domain; wherein the extracellular antigen binding domain specific to TACI asset forth in claim 2.

31. The anti-TACI CAR of claim 30, wherein the co-stimulatory signaling domain is from a co-stimulatory molecule selected from the CD28, 4-1BB, OX40, ICOS, CD27, CD40, or CD40L; and/or wherein the cytoplasmic signaling domain is from CD3ζ.

32. The anti-TACI CAR of claim 30, further comprising a hinge domain and/or a transmembrane domain between the extracellular antigen binding domain specific to TACI and the co-stimulatory domain.

33. The anti-TACI CAR of claim 32, which comprises both the hinge domain and the transmembrane domain, and wherein the anti-TACI CAR further comprises a spacer between the hinge domain and the transmembrane domain.

34. The anti-TACI CAR of claim 30, which comprises an amino acid sequence of any one of SEQ ID NOs: 143-162; optionally wherein the anti-TACI CAR comprises the amino acid sequence of SEQ ID NO: 143 or 144.

35. An anti-BCMA chimeric antigen receptor (CAR), comprising an extracellular antigen binding domain specific BCMA, a co-stimulatory signaling domain, and a cytoplasmic signaling domain; wherein the extracellular antigen binding domain specific to BCMA asset forth in claim 6.

36. The anti-BCMA CAR of claim 35, wherein the co-stimulatory signaling domain is from a co-stimulatory molecule selected from the CD28, 4-1BB, OX40, ICOS, CD27, CD40, or CD40L; and/or wherein the cytoplasmic signaling domain is from CD3ζ.

37. The anti-BCMA CAR of claim 35, further comprising a hinge domain and/or a transmembrane domain between the extracellular antigen binding domain specific to BCMA and the co-stimulatory domain.

38. The anti-BCMA CAR of claim 37, which comprises both the hinge domain and the transmembrane domain, and wherein the anti-BCMA CAR further comprises a spacer between the hinge domain and the transmembrane domain.

39. The anti-BCMA CAR of claim 35, which comprises an amino acid sequence of any one of SEQ ID NOs: 119-142; optionally wherein the anti-BCMA CAR comprises the amino acid sequence of SEQ ID NO: 127 or 128.

40. A nucleic acid, comprising a first nucleotide sequence encoding an anti-TACI CAR or an anti-BCMA CAR of claim 1.

41. The nucleic acid of claim 40, further comprising a second nucleotide sequence encoding an armor polypeptide, which enhances T cell functionality, and a third nucleotide sequence encoding a self-cleaving peptide, which is located between the first and second nucleotide sequences.

42. The nucleic acid of claim 40, wherein the armor polypeptide is IL-2, IL-5, IL-15, a co-stimulatory ligand, an anti-PDL1 antibody, or a fusion polypeptide comprising the anti-PDL1 antibody; optionally wherein the anti-PDL1 antibody is a single chain variable fragment (scFv), which is fused to an IL-2 polypeptide.

43. The nucleic acid of claim 40, wherein the nucleic acid is an expression vector, optionally a viral vector.

44. A genetically engineered immune cell, which expresses the anti-TACI CAR and/or the anti-BCMA CAR of claim 1, and optionally further expresses an armor polypeptide, which enhances T cell functionality.

45. The genetically engineered immune cell of claim 44, which is a T cell, an NK cell, or a macrophage, optionally wherein the immune cell is a T cell.

46. A method for eliminating undesired cells in a subject, the method comprising administering to a subject in need thereof an effective amount of the genetically engineered immune cell of claim 26, or a pharmaceutical composition comprising such.

47. The method of claim 46, wherein the undesired cells are cancer cells.

48. The method of claim 46, wherein the subject is a human cancer patient.

49. The method of claim 48, wherein the human cancer patient comprises BCMA⁺ and/or TACI⁺ cancer cells.

50. The method of claim 49, wherein the cancer cells are multiple myeloma cells, lung cancer cells, gastric cancer cells, breast cancer cells, or testis cancer cells.


SEQUENCE LISTING (181 sequences)

SEQ ID NO: 1 - protein, 17 aa, synthetic construct
  VARIANT 1: note = X is V or F
  VARIANT 3: note = X is S or R
  VARIANT 8: note = X is D or N
XIXYDGSXKYYADSVKG

SEQ ID NO: 2 - protein, 11 aa, synthetic construct
  VARIANT 7: note = X is N or S
QASQDIXNYLN

SEQ ID NO: 3 - protein, 10 aa, synthetic construct
  VARIANT 2: note = X is A or S
  VARIANT 3: note = X is S or T
  VARIANT 4: note = X is G or S
  VARIANT 10: note = X is G or N
RXXXISSYLX

SEQ ID NO: 4 - protein, 7 aa, synthetic construct
  VARIANT 5: note = X is L or V
DASNXET

SEQ ID NO: 5 - protein, 7 aa, synthetic construct
  VARIANT 2: note = X is A or T
  VARIANT 4: note = X is S or T
AXSXLQS

SEQ ID NO: 6 - protein, 9 aa, synthetic construct
  VARIANT 5: note = X is K or N
  VARIANT 8: note = X is F, L, or Y
QQYDXLPXT

SEQ ID NO: 7 - protein, 5 aa, synthetic construct
SYGMH

SEQ ID NO: 8 - protein, 17 aa, synthetic construct
FIRYDGSNKYYADSVKG

SEQ ID NO: 9 - protein, 13 aa, synthetic construct
DLWDGIVGAPAGY

SEQ ID NO: 10 - protein, 122 aa, synthetic construct
EVQLVQSGGGVVQPGGSLRLSCAASGFTFSSYGMHWVRQAPGKGLEWVAFIRYDGSNKYYADSVKGRFTISRDNSKNTLYLQMNSLRAEDTAVYYCAKDLWDGIVGAPAGYWGQGTLVTVSS

SEQ ID NO: 11 - protein, 11 aa, synthetic construct
QASQDISNYLN

SEQ ID NO: 12 - protein, 7 aa, synthetic construct
DASNLET

SEQ ID NO: 13 - protein, 9 aa, synthetic construct
QQYDNLPLT

SEQ ID NO: 14 - protein, 107 aa, synthetic construct
DVVMTQSPSSLSASVGDRVTITCQASQDISNYLNWYQQKPGKAPKLLIYDASNLETGVPSRFSGSGSGTDFTFTISSLQPEDIATYYCQQYDNLPLTFGGGTKVEIK

SEQ ID NO: 15 - protein, 244 aa, synthetic construct
EVQLVQSGGGVVQPGGSLRLSCAASGFTFSSYGMHWVRQAPGKGLEWVAFIRYDGSNKYYADSVKGRFTISRDNSKNTLYLQMNSLRAEDTAVYYCAKDLWDGIVGAPAGYWGQGTLVTVSSGGGGSGGGGSGGGGSDVVMTQSPSSLSASVGDRVTITCQASQDISNYLNWYQQKPGKAPKLLIYDASNLETGVPSRFSGSGSGTDFTFTISSLQPEDIATYYCQQYDNLPLTFGGGTKVEIK

SEQ ID NO: 16 - protein, 17 aa, synthetic construct
DWDYYDSSGYYPDALGI

SEQ ID NO: 17 - protein, 126 aa, synthetic construct
QVQLVQSGGGVVQPGGSLRLSCAASGFTFSSYGMHWVRQAPGKVLEWVAFIRYDGSNKYYADSVKGRFTISRDNSKNTLYLQMNSLRAEDTAVYYCAKDWDYYDSSGYYPDALGIWGQGTMVTVSS

SEQ ID NO: 18 - protein, 9 aa, synthetic construct
QQYDNLPFT

SEQ ID NO: 19 - protein, 107 aa, synthetic construct
EIVLTQSPSSLSASVGDRVTITCQASQDISNYLNWYQQKPGKAPKLLIYDASNLETGVPSRFSGSGSGTDFTFTISSLQPEDIATYYCQQYDNLPFTFGPGTKVDIK

SEQ ID NO: 20 - protein, 248 aa, synthetic construct
QVQLVQSGGGVVQPGGSLRLSCAASGFTFSSYGMHWVRQAPGKVLEWVAFIRYDGSNKYYADSVKGRFTISRDNSKNTLYLQMNSLRAEDTAVYYCAKDWDYYDSSGYYPDALGIWGQGTMVTVSSGGGGSGGGGSGGGGSEIVLTQSPSSLSASVGDRVTITCQASQDISNYLNWYQQKPGKAPKLLIYDASNLETGVPSRFSGSGSGTDFTFTISSLQPEDIATYYCQQYDNLPFTFGPGTKVDIK

SEQ ID NO: 21 - protein, 17 aa, synthetic construct
FIRYDGSDKYYADSVKG

SEQ ID NO: 22 - protein, 9 aa, synthetic construct
DLTTITPGY

SEQ ID NO: 23 - protein, 118 aa, synthetic construct
QMQLVQSGGGVVQPGGSLRLSCAASGFTFSSYGMHWVRQAPGKGLEWVAFIRYDGSDKYYADSVKGRFTISRDNSKNTLYLQMNSLRAEDTAVYYCAKDLTTITPGYWGQGTLVTVSS

SEQ ID NO: 24 - protein, 107 aa, synthetic construct
VIQMTQSPSSLSASVGDRVTITCQASQDISNYLNWYQQKPGKAPKLLIYDASNLETGVPSRFSGSGSGTDFTFTISSLQPEDIATYYCQQYDNLPLTFGGGTKVEIK

SEQ ID NO: 25 - protein, 240 aa, synthetic construct
QMQLVQSGGGVVQPGGSLRLSCAASGFTFSSYGMHWVRQAPGKGLEWVAFIRYDGSDKYYADSVKGRFTISRDNSKNTLYLQMNSLRAEDTAVYYCAKDLTTITPGYWGQGTLVTVSSGGGGSGGGGSGGGGSVIQMTQSPSSLSASVGDRVTITCQASQDISNYLNWYQQKPGKAPKLLIYDASNLETGVPSRFSGSGSGTDFTFTISSLQPEDIATYYCQQYDNLPLTFGGGTKVEIK

SEQ ID NO: 26 - protein, 5 aa, synthetic construct
DYAMS

SEQ ID NO: 27 - protein, 19 aa, synthetic construct
FIRSKAYGGTTEYAASVKG

SEQ ID NO: 28 - protein, 14 aa, synthetic construct
GPHYDILTSNWFDP

SEQ ID NO: 29 - protein, 125 aa, synthetic construct
EVQLVESGGGLVQPGRSLRLSCTASGFTFGDYAMSWFRQAPGKGLEWVGFIRSKAYGGTTEYAASVKGRFTISRDDSKSIAYLQMNSLKTEDTAVYYCTPGPHYDILTSNWFDPWGQGTLVTVSS

SEQ ID NO: 30 - protein, 11 aa, synthetic construct
RASQSISSYLN

SEQ ID NO: 31 - protein, 7 aa, synthetic construct
AASSLQS

SEQ ID NO: 32 - protein, 9 aa, synthetic construct
QHSYSTPHT

SEQ ID NO: 33 - protein, 107 aa, synthetic construct
DIVMTHSPSSLSASVGDRVTITCRASQSISSYLNWYQQKPGKAPKLLIYAASSLQSGVPSRFSGSGSGTDFTLTISSLQPEDFATYYCQHSYSTPHTFGQGTKVEIK

SEQ ID NO: 34 - protein, 247 aa, synthetic construct
EVQLVESGGGLVQPGRSLRLSCTASGFTFGDYAMSWFRQAPGKGLEWVGFIRSKAYGGTTEYAASVKGRFTISRDDSKSIAYLQMNSLKTEDTAVYYCTPGPHYDILTSNWFDPWGQGTLVTVSSGGGGSGGGGSGGGGSDIVMTHSPSSLSASVGDRVTITCRASQSISSYLNWYQQKPGKAPKLLIYAASSLQSGVPSRFSGSGSGTDFTLTISSLQPEDFATYYCQHSYSTPHTFGQGTKVEIK

SEQ ID NO: 35 - protein, 13 aa, synthetic construct
DWEDPLYYYDTPF

SEQ ID NO: 36 - protein, 122 aa, synthetic construct
QMQLVQSGGGVVQPGGSLRLSCAASGFTFSSYGMHWVRQAPGKGLEWVAFIRYDGSNKYYADSVKGRFTISRDNSKNTLYLQMNSLRAEDTAVYYCAKDWEDPLYYYDTPFWGQGTLVTVSS

SEQ ID NO: 37 - protein, 11 aa, synthetic construct
QASQDINNYLN

SEQ ID NO: 38 - protein, 7 aa, synthetic construct
DASNVET

SEQ ID NO: 39 - protein, 9 aa, synthetic construct
QQYDKLPYT

SEQ ID NO: 40 - protein, 107 aa, synthetic construct
VIWMTQSPSSVSASVGDIITITCQASQDINNYLNWYQLKPGKAPKLLIYDASNVETGVPSRFSGSGSGTDFTFTISSLQPEDTATYYCQQYDKLPYTFGQGTKLEIK

SEQ ID NO: 41 - protein, 244 aa, synthetic construct
QMQLVQSGGGVVQPGGSLRLSCAASGFTFSSYGMHWVRQAPGKGLEWVAFIRYDGSNKYYADSVKGRFTISRDNSKNTLYLQMNSLRAEDTAVYYCAKDWEDPLYYYDTPFWGQGTLVTVSSGGGGSGGGGSGGGGSVIWMTQSPSSVSASVGDIITITCQASQDINNYLNWYQLKPGKAPKLLIYDASNVETGVPSRFSGSGSGTDFTFTISSLQPEDTATYYCQQYDKLPYTFGQGTKLEIK

SEQ ID NO: 42 - protein, 5 aa, synthetic construct
DYAMH

SEQ ID NO: 43 - protein, 17 aa, synthetic construct
GISWNSGSIGYADSVKG

SEQ ID NO: 44 - protein, 9 aa, synthetic construct
VQPPGAFDI

SEQ ID NO: 45 - protein, 118 aa, synthetic construct
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKVQPPGAFDIWGQGTMVTVSS

SEQ ID NO: 46 - protein, 11 aa, synthetic construct
RSTQGISSYLG

SEQ ID NO: 47 - protein, 7 aa, synthetic construct
ATSTLQS

SEQ ID NO: 48 - protein, 5 aa, synthetic construct
QQLYS

SEQ ID NO: 49 - protein, 103 aa, synthetic construct
DIQLTQSPSFLSASVGDRVSITCRSTQGISSYLGWYQQKPGKAPKLLIYATSTLQSGVPSRFSGSGSGTEFTLTISSLQPEDFATYYCQQLYSFGQGTKLEIK

SEQ ID NO: 50 - protein, 236 aa, synthetic construct
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKVQPPGAFDIWGQGTMVTVSSGGGGSGGGGSGGGGSDIQLTQSPSFLSASVGDRVSITCRSTQGISSYLGWYQQKPGKAPKLLIYATSTLQSGVPSRFSGSGSGTEFTLTISSLQPEDFATYYCQQLYSFGQGTKLEIK

SEQ ID NO: 51 - protein, 9 aa, synthetic construct
VQSPGAFDI

SEQ ID NO: 52 - protein, 118 aa, synthetic construct
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKVQSPGAFDIWGQGTMVTVSS

SEQ ID NO: 53 - protein, 236 aa, synthetic construct
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKVQSPGAFDIWGQGTMVTVSSGGGGSGGGGSGGGGSDIQLTQSPSFLSASVGDRVSITCRSTQGISSYLGWYQQKPGKAPKLLIYATSTLQSGVPSRFSGSGSGTEFTLTISSLQPEDFATYYCQQLYSFGQGTKLEIK

SEQ ID NO: 54 - protein, 5 aa, synthetic construct
SYAMH

SEQ ID NO: 55 - protein, 17 aa, synthetic construct
VISYDGSNKYYADSVKG

SEQ ID NO: 56 - protein, 13 aa, synthetic construct
DEHQVVPNYRFDF

SEQ ID NO: 57 - protein, 122 aa, synthetic construct
QVQLVQSGGGVVQPGRSLRLSCAASGFTFSSYAMHWVRQAPGKGLEWVAVISYDGSNKYYADSVKGRFTISRDNAENSVYLQMNSLRAEDTAVYYCARDEHQVVPNYRFDFWGQGSQVTVSS

SEQ ID NO: 58 - protein, 13 aa, synthetic construct
SGSGSNIGSNDVS

SEQ ID NO: 59 - protein, 7 aa, synthetic construct
WNDQRPS

SEQ ID NO: 60 - protein, 11 aa, synthetic construct
AAWDDSLNGWV

SEQ ID NO: 61 - protein, 110 aa, synthetic construct
QLVLTQPPSASGTPGQRVTISCSGSGSNIGSNDVSWYQQIPGTASKLLIYWNDQRPSGVPDRFSGSKSGTSASLAISGLQSEDEADYYCAAWDDSLNGWVFGGGTKLTVL

SEQ ID NO: 62 - protein, 247 aa, synthetic construct
QVQLVQSGGGVVQPGRSLRLSCAASGFTFSSYAMHWVRQAPGKGLEWVAVISYDGSNKYYADSVKGRFTISRDNAENSVYLQMNSLRAEDTAVYYCARDEHQVVPNYRFDFWGQGSQVTVSSGGGGSGGGGSGGGGSQLVLTQPPSASGTPGQRVTISCSGSGSNIGSNDVSWYQQIPGTASKLLIYWNDQRPSGVPDRFSGSKSGTSASLAISGLQSEDEADYYCAAWDDSLNGWVFGGGTKLTVL

SEQ ID NO: 63 - protein, 12 aa, synthetic construct
DLWEFGGDYADY

SEQ ID NO: 64 - protein, 121 aa, synthetic construct
QVQLVQSGGGVVQPGGSLRLSCAASGFTFSSYGMHWVRQAPGKGLEWVAFIRYDGSNKYYADSVKGRFTISRDNSKNTLYLQMNSLRAEDTAVYYCAKDLWEFGGDYADYWGQGTLVTVSS

SEQ ID NO: 65 - protein, 107 aa, synthetic construct
NIQMTQSPSSLSASVGDRVTITCQASQDISNYLNWYQQKPGKAPKLLIYDASNLETGVPSRFSGSGSGTDFTFTISSLQPEDIATYYCQQYDNLPLTFGGGTKVEIK

SEQ ID NO: 66 - protein, 243 aa, synthetic construct
QVQLVQSGGGVVQPGGSLRLSCAASGFTFSSYGMHWVRQAPGKGLEWVAFIRYDGSNKYYADSVKGRFTISRDNSKNTLYLQMNSLRAEDTAVYYCAKDLWEFGGDYADYWGQGTLVTVSSGGGGSGGGGSGGGGSNIQMTQSPSSLSASVGDRVTITCQASQDISNYLNWYQQKPGKAPKLLIYDASNLETGVPSRFSGSGSGTDFTFTISSLQPEDIATYYCQQYDNLPLTFGGGTKVEIK

SEQ ID NO: 67 - protein, 5 aa, synthetic construct
GYFMH

SEQ ID NO: 68 - protein, 17 aa, synthetic construct
WINSNSGGTNYAQKFQG

SEQ ID NO: 69 - protein, 11 aa, synthetic construct
DDGTGYNAFDI

SEQ ID NO: 70 - protein, 120 aa, synthetic construct
QVQLVQSGAEVKKPGASVKVSCKASGYTFTGYFMHWVRQAPGQGLEWMGWINSNSGGTNYAQKFQGRVTMTRDTSISTAYMELSRLRSDDTAVYYCARDDGTGYNAFDIWGQGTMVTVSS

SEQ ID NO: 71 - protein, 16 aa, synthetic construct
RSSQSLLHSNGYNYLD

SEQ ID NO: 72 - protein, 7 aa, synthetic construct
LASHRAS

SEQ ID NO: 73 - protein, 8 aa, synthetic construct
MQAQQTAL

SEQ ID NO: 74 - protein, 111 aa, synthetic construct
DIVMTQSPLSLPVTPGEPASISCRSSQSLLHSNGYNYLDWYVQKPGKPPQLLIYLASHRASGVPDRFSGSGSGTDFTLKISRVEAEDVGIYYCMQAQQTALFGPGTKVDIK

SEQ ID NO: 75 - protein, 246 aa, synthetic construct
QVQLVQSGAEVKKPGASVKVSCKASGYTFTGYFMHWVRQAPGQGLEWMGWINSNSGGTNYAQKFQGRVTMTRDTSISTAYMELSRLRSDDTAVYYCARDDGTGYNAFDIWGQGTMVTVSSGGGGSGGGGSGGGGSDIVMTQSPLSLPVTPGEPASISCRSSQSLLHSNGYNYLDWYVQKPGKPPQLLIYLASHRASGVPDRFSGSGSGTDFTLKISRVEAEDVGIYYCMQAQQTALFGPGTKVDIK

SEQ ID NO: 76 - protein, 5 aa, synthetic construct
GYYWS

SEQ ID NO: 77 - protein, 16 aa, synthetic construct
EINHSGSTNYNPSLKS

SEQ ID NO: 78 - protein, 11 aa, synthetic construct
IRKLGGYYFDY

SEQ ID NO: 79 - protein, 119 aa, synthetic construct
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARIRKLGGYYFDYWGQGTLVTVSS

SEQ ID NO: 80 - protein, 12 aa, synthetic construct
RASQSVSSSYLA

SEQ ID NO: 81 - protein, 7 aa, synthetic construct
GASSRAT

SEQ ID NO: 82 - protein, 9 aa, synthetic construct
QQYGSSPRP

SEQ ID NO: 83 - protein, 108 aa, synthetic construct
EIVMTQSPGTLSLSPGERATLSCRASQSVSSSYLAWYQQKPGQAPRLLIYGASSRATGIPDRFSGSGSGTDFTLTISRLEPEDFAVYYCQQYGSSPRPFGQGTKVEIK

SEQ ID NO: 84 - protein, 242 aa, synthetic construct
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARIRKLGGYYFDYWGQGTLVTVSSGGGGSGGGGSGGGGSEIVMTQSPGTLSLSPGERATLSCRASQSVSSSYLAWYQQKPGQAPRLLIYGASSRATGIPDRFSGSGSGTDFTLTISRLEPEDFAVYYCQQYGSSPRPFGQGTKVEIK

SEQ ID NO: 85 - protein, 5 aa, synthetic construct
SDYMS

SEQ ID NO: 86 - protein, 16 aa, synthetic construct
FIYRGGSTYYADSVKG

SEQ ID NO: 87 - protein, 8 aa, synthetic construct
GLGSSSDY

SEQ ID NO: 88 - protein, 116 aa, synthetic construct
EVQLVESGGGLVQPGGSLRLSCATSGFTVNSDYMSWVRQSPEKGLEWVSFIYRGGSTYYADSVKGRFTVSRDTSKNTLYLQMNSLRVEDTAVYYCARGLGSSSDYWGQGTLVTVSS

SEQ ID NO: 89 - protein, 13 aa, synthetic construct
TRSSGSIANNYVQ

SEQ ID NO: 90 - protein, 7 aa, synthetic construct
EDDHRPS

SEQ ID NO: 91 - protein, 10 aa, synthetic construct
QSYDGIDHYV

SEQ ID NO: 92 - protein, 111 aa, synthetic construct
SYELTQPHSVSESPGKTVTISCTRSSGSIANNYVQWYRHRPGSAPSTVIFEDDHRPSEVPDRFSGSIDSSSNSASLTISGLKTEDEADYYCQSYDGIDHYVFGTGTKLTVL

SEQ ID NO: 93 - protein, 242 aa, synthetic construct
EVQLVESGGGLVQPGGSLRLSCATSGFTVNSDYMSWVRQSPEKGLEWVSFIYRGGSTYYADSVKGRFTVSRDTSKNTLYLQMNSLRVEDTAVYYCARGLGSSSDYWGQGTLVTVSSGGGGSGGGGSGGGGSSYELTQPHSVSESPGKTVTISCTRSSGSIANNYVQWYRHRPGSAPSTVIFEDDHRPSEVPDRFSGSIDSSSNSASLTISGLKTEDEADYYCQSYDGIDHYVFGTGTKLTVL

SEQ ID NO: 94 - protein, 5 aa, synthetic construct
SYAIS

SEQ ID NO: 95 - protein, 17 aa, synthetic construct
GIIPIFGTANYAQKFQG

SEQ ID NO: 96 - protein, 12 aa, synthetic construct
DKANTALRWFDP

SEQ ID NO: 97 - protein, 121 aa, synthetic construct
EVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARDKANTALRWFDPWGQGTLVTVSS

SEQ ID NO: 98 - protein, 12 aa, synthetic construct
RASQRVSSSYLA

SEQ ID NO: 99 - protein, 7 aa, synthetic construct
GASKRAT

SEQ ID NO: 100 - protein, 9 aa, synthetic construct
QQYGDSPLT

SEQ ID NO: 101 - protein, 108 aa, synthetic construct
EIVMTQSPGSLSLSPGDTATLSCRASQRVSSSYLAWYQQKPGQAPRLLIYGASKRATGISSKFSGSGSETDFLLTISRLEPEDIAVYYCQQYGDSPLTFGQGTKVEIK

SEQ ID NO: 102 - protein, 244 aa, synthetic construct
EVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARDKANTALRWFDPWGQGTLVTVSSGGGGSGGGGSGGGGSEIVMTQSPGSLSLSPGDTATLSCRASQRVSSSYLAWYQQKPGQAPRLLIYGASKRATGISSKFSGSGSETDFLLTISRLEPEDIAVYYCQQYGDSPLTFGQGTKVEIK

SEQ ID NO: 103 - protein, 22 aa, synthetic construct
MLLLVTSLLLCELPHPAFLLIP

SEQ ID NO: 104 - protein, 5 aa, synthetic construct
GGGGS

SEQ ID NO: 105 - protein, 10 aa, synthetic construct
GGGGSGGGGS

SEQ ID NO: 106 - protein, 15 aa, synthetic construct
GGGGSGGGGSGGGGS

SEQ ID NO: 107 - protein, 18 aa, synthetic construct
GSTSGSGKPGSGEGSTKG

SEQ ID NO: 108 - protein, 8 aa, synthetic construct
DYKDDDDK

SEQ ID NO: 109 - protein, 12 aa, synthetic construct
ESKYGPPCPPCP

SEQ ID NO: 110 - protein, 105 aa, synthetic construct
GQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQESLSLSP

SEQ ID NO: 111 - protein, 215 aa, synthetic construct
APEFEGAPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQESLSLSP

SEQ ID NO: 112 - protein, 28 aa, synthetic construct
MFWVLVVVGGVLACYSLLVTVAFIIFWV

SEQ ID NO: 113 - protein, 24 aa, synthetic construct
LEGGGEGRGSLLTCGDVEENPGPR

SEQ ID NO: 114 - protein, 21 aa, synthetic construct
VSFCLVMVLLFAVDTGLYFSV

SEQ ID NO: 115 - protein, 27 aa, synthetic construct
FWVLVVVGGVLACYSLLVTVAFIIFWV

SEQ ID NO: 116 - protein, 42 aa, synthetic construct
KRGRKKLLYIFKQPFMRPVQTTQEEDGCSCRFPEEEEGGCEL

SEQ ID NO: 117 - protein, 41 aa, synthetic construct
RSKRSRLLHSDYMFMTPRRPGPTRKHYQPYAPPRDFAAYRS

SEQ ID NO: 118 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 119 - protein, 557 aa, synthetic construct
MLLLVTSLLLCELPHPAFLLIPEVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKVQPPGAFDIWGQGTMVTVSSGGGGSGGGGSGGGGSDIQLTQSPSFLSASVGDRVSITCRSTQGISSYLGWYQQKPGKAPKLLIYATSTLQSGVPSRFSGSGSGTEFTLTISSLQPEDFATYYCQQLYSFGQGTKLEIKGSESKYGPPCPPCPPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQESLSLSPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 120 - protein, 535 aa, synthetic construct
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKVQPPGAFDIWGQGTMVTVSSGGGGSGGGGSGGGGSDIQLTQSPSFLSASVGDRVSITCRSTQGISSYLGWYQQKPGKAPKLLIYATSTLQSGVPSRFSGSGSGTEFTLTISSLQPEDFATYYCQQLYSFGQGTKLEIKGSESKYGPPCPPCPPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQESLSLSPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 121 - protein, 565 aa, synthetic construct
MLLLVTSLLLCELPHPAFLLIPEVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKVQPPGAFDIWGQGTMVTVSSGGGGSGGGGSGGGGSDIQLTQSPSFLSASVGDRVSITCRSTQGISSYLGWYQQKPGKAPKLLIYATSTLQSGVPSRFSGSGSGTEFTLTISSLQPEDFATYYCQQLYSFGQGTKLEIKGSDYKDDDDKESKYGPPCPPCPPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQESLSLSPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 122 - protein, 543 aa, synthetic construct
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKVQPPGAFDIWGQGTMVTVSSGGGGSGGGGSGGGGSDIQLTQSPSFLSASVGDRVSITCRSTQGISSYLGWYQQKPGKAPKLLIYATSTLQSGVPSRFSGSGSGTEFTLTISSLQPEDFATYYCQQLYSFGQGTKLEIKGSDYKDDDDKESKYGPPCPPCPPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQESLSLSPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 123 - protein, 454 aa, synthetic construct
MLLLVTSLLLCELPHPAFLLIPEVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKVQPPGAFDIWGQGTMVTVSSGGGGSGGGGSGGGGSDIQLTQSPSFLSASVGDRVSITCRSTQGISSYLGWYQQKPGKAPKLLIYATSTLQSGVPSRFSGSGSGTEFTLTISSLQPEDFATYYCQQLYSFGQGTKLEIKGSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 124 - protein, 432 aa, synthetic construct
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKVQPPGAFDIWGQGTMVTVSSGGGGSGGGGSGGGGSDIQLTQSPSFLSASVGDRVSITCRSTQGISSYLGWYQQKPGKAPKLLIYATSTLQSGVPSRFSGSGSGTEFTLTISSLQPEDFATYYCQQLYSFGQGTKLEIKGSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 125 - protein, 462 aa, synthetic construct
MLLLVTSLLLCELPHPAFLLIPEVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKVQPPGAFDIWGQGTMVTVSSGGGGSGGGGSGGGGSDIQLTQSPSFLSASVGDRVSITCRSTQGISSYLGWYQQKPGKAPKLLIYATSTLQSGVPSRFSGSGSGTEFTLTISSLQPEDFATYYCQQLYSFGQGTKLEIKGSDYKDDDDKESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 126 - protein, 440 aa, synthetic construct
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKVQPPGAFDIWGQGTMVTVSSGGGGSGGGGSGGGGSDIQLTQSPSFLSASVGDRVSITCRSTQGISSYLGWYQQKPGKAPKLLIYATSTLQSGVPSRFSGSGSGTEFTLTISSLQPEDFATYYCQQLYSFGQGTKLEIKGSDYKDDDDKESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 127 - protein, 559 aa, synthetic construct
MLLLVTSLLLCELPHPAFLLIPEVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKVQPPGAFDIWGQGTMVTVSSGGGGSGGGGSGGGGSDIQLTQSPSFLSASVGDRVSITCRSTQGISSYLGWYQQKPGKAPKLLIYATSTLQSGVPSRFSGSGSGTEFTLTISSLQPEDFATYYCQQLYSFGQGTKLEIKGSESKYGPPCPPCPGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQESLSLSPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 128 - protein, 537 aa, synthetic construct
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKVQPPGAFDIWGQGTMVTVSSGGGGSGGGGSGGGGSDIQLTQSPSFLSASVGDRVSITCRSTQGISSYLGWYQQKPGKAPKLLIYATSTLQSGVPSRFSGSGSGTEFTLTISSLQPEDFATYYCQQLYSFGQGTKLEIKGSESKYGPPCPPCPGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQESLSLSPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 129 - protein, 567 aa, synthetic construct
MLLLVTSLLLCELPHPAFLLIPEVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKVQPPGAFDIWGQGTMVTVSSGGGGSGGGGSGGGGSDIQLTQSPSFLSASVGDRVSITCRSTQGISSYLGWYQQKPGKAPKLLIYATSTLQSGVPSRFSGSGSGTEFTLTISSLQPEDFATYYCQQLYSFGQGTKLEIKGSDYKDDDDKESKYGPPCPPCPGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQESLSLSPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 130 - protein, 545 aa, synthetic construct
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKVQPPGAFDIWGQGTMVTVSSGGGGSGGGGSGGGGSDIQLTQSPSFLSASVGDRVSITCRSTQGISSYLGWYQQKPGKAPKLLIYATSTLQSGVPSRFSGSGSGTEFTLTISSLQPEDFATYYCQQLYSFGQGTKLEIKGSDYKDDDDKESKYGPPCPPCPGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQESLSLSPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 131 - protein, 669 aa, synthetic construct
MLLLVTSLLLCELPHPAFLLIPEVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKVQPPGAFDIWGQGTMVTVSSGGGGSGGGGSGGGGSDIQLTQSPSFLSASVGDRVSITCRSTQGISSYLGWYQQKPGKAPKLLIYATSTLQSGVPSRFSGSGSGTEFTLTISSLQPEDFATYYCQQLYSFGQGTKLEIKGSESKYGPPCPPCPAPEFEGAPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQESLSLSPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 132 - protein, 647 aa, synthetic construct
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKVQPPGAFDIWGQGTMVTVSSGGGGSGGGGSGGGGSDIQLTQSPSFLSASVGDRVSITCRSTQGISSYLGWYQQKPGKAPKLLIYATSTLQSGVPSRFSGSGSGTEFTLTISSLQPEDFATYYCQQLYSFGQGTKLEIKGSESKYGPPCPPCPAPEFEGAPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQESLSLSPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 133 - protein, 677 aa, synthetic construct
MLLLVTSLLLCELPHPAFLLIPEVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKVQPPGAFDIWGQGTMVTVSSGGGGSGGGGSGGGGSDIQLTQSPSFLSASVGDRVSITCRSTQGISSYLGWYQQKPGKAPKLLIYATSTLQSGVPSRFSGSGSGTEFTLTISSLQPEDFATYYCQQLYSFGQGTKLEIKGSDYKDDDDKESKYGPPCPPCPAPEFEGAPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQESLSLSPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 134 - protein, 655 aa, synthetic construct
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKVQPPGAFDIWGQGTMVTVSSGGGGSGGGGSGGGGSDIQLTQSPSFLSASVGDRVSITCRSTQGISSYLGWYQQKPGKAPKLLIYATSTLQSGVPSRFSGSGSGTEFTLTISSLQPEDFATYYCQQLYSFGQGTKLEIKGSDYKDDDDKESKYGPPCPPCPAPEFEGAPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQESLSLSPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 135 - protein, 570 aa, synthetic construct
MLLLVTSLLLCELPHPAFLLIPQVQLVQSGGGVVQPGRSLRLSCAASGFTFSSYAMHWVRQAPGKGLEWVAVISYDGSNKYYADSVKGRFTISRDNAENSVYLQMNSLRAEDTAVYYCARDEHQVVPNYRFDFWGQGSQVTVSSGGGGSGGGGSGGGGSQLVLTQPPSASGTPGQRVTISCSGSGSNIGSNDVSWYQQIPGTASKLLIYWNDQRPSGVPDRFSGSKSGTSASLAISGLQSEDEADYYCAAWDDSLNGWVFGGGTKLTVLGSESKYGPPCPPCPGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQESLSLSPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 136 - protein, 548 aa, synthetic construct
QVQLVQSGGGVVQPGRSLRLSCAASGFTFSSYAMHWVRQAPGKGLEWVAVISYDGSNKYYADSVKGRFTISRDNAENSVYLQMNSLRAEDTAVYYCARDEHQVVPNYRFDFWGQGSQVTVSSGGGGSGGGGSGGGGSQLVLTQPPSASGTPGQRVTISCSGSGSNIGSNDVSWYQQIPGTASKLLIYWNDQRPSGVPDRFSGSKSGTSASLAISGLQSEDEADYYCAAWDDSLNGWVFGGGTKLTVLGSESKYGPPCPPCPGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQESLSLSPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 137 - protein, 578 aa, synthetic construct
MLLLVTSLLLCELPHPAFLLIPQVQLVQSGGGVVQPGRSLRLSCAASGFTFSSYAMHWVRQAPGKGLEWVAVISYDGSNKYYADSVKGRFTISRDNAENSVYLQMNSLRAEDTAVYYCARDEHQVVPNYRFDFWGQGSQVTVSSGGGGSGGGGSGGGGSQLVLTQPPSASGTPGQRVTISCSGSGSNIGSNDVSWYQQIPGTASKLLIYWNDQRPSGVPDRFSGSKSGTSASLAISGLQSEDEADYYCAAWDDSLNGWVFGGGTKLTVLGSDYKDDDDKESKYGPPCPPCPGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQESLSLSPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 138 - protein, 556 aa, synthetic construct
QVQLVQSGGGVVQPGRSLRLSCAASGFTFSSYAMHWVRQAPGKGLEWVAVISYDGSNKYYADSVKGRFTISRDNAENSVYLQMNSLRAEDTAVYYCARDEHQVVPNYRFDFWGQGSQVTVSSGGGGSGGGGSGGGGSQLVLTQPPSASGTPGQRVTISCSGSGSNIGSNDVSWYQQIPGTASKLLIYWNDQRPSGVPDRFSGSKSGTSASLAISGLQSEDEADYYCAAWDDSLNGWVFGGGTKLTVLGSDYKDDDDKESKYGPPCPPCPGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQESLSLSPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 139 - protein, 567 aa, synthetic construct
MLLLVTSLLLCELPHPAFLLIPQVQLVQSGGGVVQPGGSLRLSCAASGFTFSSYGMHWVRQAPGKGLEWVAFIRYDGSNKYYADSVKGRFTISRDNSKNTLYLQMNSLRAEDTAVYYCAKDWEDPLYYYDTPFWGQGTLVTVSSGGGGSGGGGSGGGGSVIWMTQSPSSVSASVGDIITITCQASQDINNYLNWYQLKPGKAPKLLIYDASNVETGVPSRFSGSGSGTDFTFTISSLQPEDTATYYCQQYDKLPYTFGQGTKLEIKGSESKYGPPCPPCPGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQESLSLSPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 140 - protein, 545 aa, synthetic construct
QVQLVQSGGGVVQPGGSLRLSCAASGFTFSSYGMHWVRQAPGKGLEWVAFIRYDGSNKYYADSVKGRFTISRDNSKNTLYLQMNSLRAEDTAVYYCAKDWEDPLYYYDTPFWGQGTLVTVSSGGGGSGGGGSGGGGSVIWMTQSPSSVSASVGDIITITCQASQDINNYLNWYQLKPGKAPKLLIYDASNVETGVPSRFSGSGSGTDFTFTISSLQPEDTATYYCQQYDKLPYTFGQGTKLEIKGSESKYGPPCPPCPGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQESLSLSPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 141 - protein, 575 aa, synthetic construct
MLLLVTSLLLCELPHPAFLLIPQVQLVQSGGGVVQPGGSLRLSCAASGFTFSSYGMHWVRQAPGKGLEWVAFIRYDGSNKYYADSVKGRFTISRDNSKNTLYLQMNSLRAEDTAVYYCAKDWEDPLYYYDTPFWGQGTLVTVSSGGGGSGGGGSGGGGSVIWMTQSPSSVSASVGDIITITCQASQDINNYLNWYQLKPGKAPKLLIYDASNVETGVPSRFSGSGSGTDFTFTISSLQPEDTATYYCQQYDKLPYTFGQGTKLEIKGSDYKDDDDKESKYGPPCPPCPGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQESLSLSPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 142 - protein, 553 aa, synthetic construct
QVQLVQSGGGVVQPGGSLRLSCAASGFTFSSYGMHWVRQAPGKGLEWVAFIRYDGSNKYYADSVKGRFTISRDNSKNTLYLQMNSLRAEDTAVYYCAKDWEDPLYYYDTPFWGQGTLVTVSSGGGGSGGGGSGGGGSVIWMTQSPSSVSASVGDIITITCQASQDINNYLNWYQLKPGKAPKLLIYDASNVETGVPSRFSGSGSGTDFTFTISSLQPEDTATYYCQQYDKLPYTFGQGTKLEIKGSDYKDDDDKESKYGPPCPPCPGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQESLSLSPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 143 - protein, 464 aa, synthetic construct
MLLLVTSLLLCELPHPAFLLIPQVQLVQSGAEVKKPGASVKVSCKASGYTFTGYFMHWVRQAPGQGLEWMGWINSNSGGTNYAQKFQGRVTMTRDTSISTAYMELSRLRSDDTAVYYCARDDGTGYNAFDIWGQGTMVTVSSGGGGSGGGGSGGGGSDIVMTQSPLSLPVTPGEPASISCRSSQSLLHSNGYNYLDWYVQKPGKPPQLLIYLASHRASGVPDRFSGSGSGTDFTLKISRVEAEDVGIYYCMQAQQTALFGPGTKVDIKGSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 144 - protein, 442 aa, synthetic construct
QVQLVQSGAEVKKPGASVKVSCKASGYTFTGYFMHWVRQAPGQGLEWMGWINSNSGGTNYAQKFQGRVTMTRDTSISTAYMELSRLRSDDTAVYYCARDDGTGYNAFDIWGQGTMVTVSSGGGGSGGGGSGGGGSDIVMTQSPLSLPVTPGEPASISCRSSQSLLHSNGYNYLDWYVQKPGKPPQLLIYLASHRASGVPDRFSGSGSGTDFTLKISRVEAEDVGIYYCMQAQQTALFGPGTKVDIKGSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 145 - protein, 472 aa, synthetic construct
MLLLVTSLLLCELPHPAFLLIPQVQLVQSGAEVKKPGASVKVSCKASGYTFTGYFMHWVRQAPGQGLEWMGWINSNSGGTNYAQKFQGRVTMTRDTSISTAYMELSRLRSDDTAVYYCARDDGTGYNAFDIWGQGTMVTVSSGGGGSGGGGSGGGGSDIVMTQSPLSLPVTPGEPASISCRSSQSLLHSNGYNYLDWYVQKPGKPPQLLIYLASHRASGVPDRFSGSGSGTDFTLKISRVEAEDVGIYYCMQAQQTALFGPGTKVDIKGSDYKDDDDKESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 146 - protein, 450 aa, synthetic construct
QVQLVQSGAEVKKPGASVKVSCKASGYTFTGYFMHWVRQAPGQGLEWMGWINSNSGGTNYAQKFQGRVTMTRDTSISTAYMELSRLRSDDTAVYYCARDDGTGYNAFDIWGQGTMVTVSSGGGGSGGGGSGGGGSDIVMTQSPLSLPVTPGEPASISCRSSQSLLHSNGYNYLDWYVQKPGKPPQLLIYLASHRASGVPDRFSGSGSGTDFTLKISRVEAEDVGIYYCMQAQQTALFGPGTKVDIKGSDYKDDDDKESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 147 - protein, 569 aa, synthetic construct
MLLLVTSLLLCELPHPAFLLIPQVQLVQSGAEVKKPGASVKVSCKASGYTFTGYFMHWVRQAPGQGLEWMGWINSNSGGTNYAQKFQGRVTMTRDTSISTAYMELSRLRSDDTAVYYCARDDGTGYNAFDIWGQGTMVTVSSGGGGSGGGGSGGGGSDIVMTQSPLSLPVTPGEPASISCRSSQSLLHSNGYNYLDWYVQKPGKPPQLLIYLASHRASGVPDRFSGSGSGTDFTLKISRVEAEDVGIYYCMQAQQTALFGPGTKVDIKGSESKYGPPCPPCPGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQESLSLSPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 148 - protein, 547 aa, synthetic construct
QVQLVQSGAEVKKPGASVKVSCKASGYTFTGYFMHWVRQAPGQGLEWMGWINSNSGGTNYAQKFQGRVTMTRDTSISTAYMELSRLRSDDTAVYYCARDDGTGYNAFDIWGQGTMVTVSSGGGGSGGGGSGGGGSDIVMTQSPLSLPVTPGEPASISCRSSQSLLHSNGYNYLDWYVQKPGKPPQLLIYLASHRASGVPDRFSGSGSGTDFTLKISRVEAEDVGIYYCMQAQQTALFGPGTKVDIKGSESKYGPPCPPCPGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQESLSLSPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 149 - protein, 577 aa, synthetic construct
MLLLVTSLLLCELPHPAFLLIPQVQLVQSGAEVKKPGASVKVSCKASGYTFTGYFMHWVRQAPGQGLEWMGWINSNSGGTNYAQKFQGRVTMTRDTSISTAYMELSRLRSDDTAVYYCARDDGTGYNAFDIWGQGTMVTVSSGGGGSGGGGSGGGGSDIVMTQSPLSLPVTPGEPASISCRSSQSLLHSNGYNYLDWYVQKPGKPPQLLIYLASHRASGVPDRFSGSGSGTDFTLKISRVEAEDVGIYYCMQAQQTALFGPGTKVDIKGSDYKDDDDKESKYGPPCPPCPGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQESLSLSPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 150 - protein, 555 aa, synthetic construct
QVQLVQSGAEVKKPGASVKVSCKASGYTFTGYFMHWVRQAPGQGLEWMGWINSNSGGTNYAQKFQGRVTMTRDTSISTAYMELSRLRSDDTAVYYCARDDGTGYNAFDIWGQGTMVTVSSGGGGSGGGGSGGGGSDIVMTQSPLSLPVTPGEPASISCRSSQSLLHSNGYNYLDWYVQKPGKPPQLLIYLASHRASGVPDRFSGSGSGTDFTLKISRVEAEDVGIYYCMQAQQTALFGPGTKVDIKGSDYKDDDDKESKYGPPCPPCPGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQESLSLSPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 151 - protein, 460 aa, synthetic construct
MLLLVTSLLLCELPHPAFLLIPEVQLVESGGGLVQPGGSLRLSCATSGFTVNSDYMSWVRQSPEKGLEWVSFIYRGGSTYYADSVKGRFTVSRDTSKNTLYLQMNSLRVEDTAVYYCARGLGSSSDYWGQGTLVTVSSGGGGSGGGGSGGGGSSYELTQPHSVSESPGKTVTISCTRSSGSIANNYVQWYRHRPGSAPSTVIFEDDHRPSEVPDRFSGSIDSSSNSASLTISGLKTEDEADYYCQSYDGIDHYVFGTGTKLTVLGSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 152 - protein, 438 aa, synthetic construct
EVQLVESGGGLVQPGGSLRLSCATSGFTVNSDYMSWVRQSPEKGLEWVSFIYRGGSTYYADSVKGRFTVSRDTSKNTLYLQMNSLRVEDTAVYYCARGLGSSSDYWGQGTLVTVSSGGGGSGGGGSGGGGSSYELTQPHSVSESPGKTVTISCTRSSGSIANNYVQWYRHRPGSAPSTVIFEDDHRPSEVPDRFSGSIDSSSNSASLTISGLKTEDEADYYCQSYDGIDHYVFGTGTKLTVLGSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 153 - protein, 468 aa, synthetic construct
MLLLVTSLLLCELPHPAFLLIPEVQLVESGGGLVQPGGSLRLSCATSGFTVNSDYMSWVRQSPEKGLEWVSFIYRGGSTYYADSVKGRFTVSRDTSKNTLYLQMNSLRVEDTAVYYCARGLGSSSDYWGQGTLVTVSSGGGGSGGGGSGGGGSSYELTQPHSVSESPGKTVTISCTRSSGSIANNYVQWYRHRPGSAPSTVIFEDDHRPSEVPDRFSGSIDSSSNSASLTISGLKTEDEADYYCQSYDGIDHYVFGTGTKLTVLGSDYKDDDDKESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 154 - protein, 446 aa, synthetic construct
EVQLVESGGGLVQPGGSLRLSCATSGFTVNSDYMSWVRQSPEKGLEWVSFIYRGGSTYYADSVKGRFTVSRDTSKNTLYLQMNSLRVEDTAVYYCARGLGSSSDYWGQGTLVTVSSGGGGSGGGGSGGGGSSYELTQPHSVSESPGKTVTISCTRSSGSIANNYVQWYRHRPGSAPSTVIFEDDHRPSEVPDRFSGSIDSSSNSASLTISGLKTEDEADYYCQSYDGIDHYVFGTGTKLTVLGSDYKDDDDKESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 155 - protein, 462 aa, synthetic construct
MLLLVTSLLLCELPHPAFLLIPEVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARDKANTALRWFDPWGQGTLVTVSSGGGGSGGGGSGGGGSEIVMTQSPGSLSLSPGDTATLSCRASQRVSSSYLAWYQQKPGQAPRLLIYGASKRATGISSKFSGSGSETDFLLTISRLEPEDIAVYYCQQYGDSPLTFGQGTKVEIKGSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 156 - protein, 440 aa, synthetic construct
EVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARDKANTALRWFDPWGQGTLVTVSSGGGGSGGGGSGGGGSEIVMTQSPGSLSLSPGDTATLSCRASQRVSSSYLAWYQQKPGQAPRLLIYGASKRATGISSKFSGSGSETDFLLTISRLEPEDIAVYYCQQYGDSPLTFGQGTKVEIKGSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 157 - protein, 470 aa, synthetic construct
MLLLVTSLLLCELPHPAFLLIPEVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARDKANTALRWFDPWGQGTLVTVSSGGGGSGGGGSGGGGSEIVMTQSPGSLSLSPGDTATLSCRASQRVSSSYLAWYQQKPGQAPRLLIYGASKRATGISSKFSGSGSETDFLLTISRLEPEDIAVYYCQQYGDSPLTFGQGTKVEIKGSDYKDDDDKESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 158 - protein, 448 aa, synthetic construct
EVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARDKANTALRWFDPWGQGTLVTVSSGGGGSGGGGSGGGGSEIVMTQSPGSLSLSPGDTATLSCRASQRVSSSYLAWYQQKPGQAPRLLIYGASKRATGISSKFSGSGSETDFLLTISRLEPEDIAVYYCQQYGDSPLTFGQGTKVEIKGSDYKDDDDKESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 159 - protein, 460 aa, synthetic construct
MLLLVTSLLLCELPHPAFLLIPQVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARIRKLGGYYFDYWGQGTLVTVSSGGGGSGGGGSGGGGSEIVMTQSPGTLSLSPGERATLSCRASQSVSSSYLAWYQQKPGQAPRLLIYGASSRATGIPDRFSGSGSGTDFTLTISRLEPEDFAVYYCQQYGSSPRPFGQGTKVEIKGSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 160 - protein, 438 aa, synthetic construct
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARIRKLGGYYFDYWGQGTLVTVSSGGGGSGGGGSGGGGSEIVMTQSPGTLSLSPGERATLSCRASQSVSSSYLAWYQQKPGQAPRLLIYGASSRATGIPDRFSGSGSGTDFTLTISRLEPEDFAVYYCQQYGSSPRPFGQGTKVEIKGSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 161 - protein, 468 aa, synthetic construct
MLLLVTSLLLCELPHPAFLLIPQVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARIRKLGGYYFDYWGQGTLVTVSSGGGGSGGGGSGGGGSEIVMTQSPGTLSLSPGERATLSCRASQSVSSSYLAWYQQKPGQAPRLLIYGASSRATGIPDRFSGSGSGTDFTLTISRLEPEDFAVYYCQQYGSSPRPFGQGTKVEIKGSDYKDDDDKESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 162 - protein, 446 aa, synthetic construct
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARIRKLGGYYFDYWGQGTLVTVSSGGGGSGGGGSGGGGSEIVMTQSPGTLSLSPGERATLSCRASQSVSSSYLAWYQQKPGQAPRLLIYGASSRATGIPDRFSGSGSGTDFTLTISRLEPEDFAVYYCQQYGSSPRPFGQGTKVEIKGSDYKDDDDKESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 163 - protein, 509 aa, synthetic construct
MLLLVTSLLLCELPHPAFLLIPEVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKVQPPGAFDIWGQGTMVTVSSGGGGSGGGGSGGGGSDIQLTQSPSFLSASVGDRVSITCRSTQGISSYLGWYQQKPGKAPKLLIYATSTLQSGVPSRFSGSGSGTEFTLTISSLQPEDFATYYCQQLYSFGQGTKLEIKGGGGSQVQLVQSGAEVKKPGASVKVSCKASGYTFTGYFMHWVRQAPGQGLEWMGWINSNSGGTNYAQKFQGRVTMTRDTSISTAYMELSRLRSDDTAVYYCARDDGTGYNAFDIWGQGTMVTVSSGGGGSGGGGSGGGGSDIVMTQSPLSLPVTPGEPASISCRSSQSLLHSNGYNYLDWYVQKPGKPPQLLIYLASHRASGVPDRFSGSGSGTDFTLKISRVEAEDVGIYYCMQAQQTALFGPGTKVDIK

SEQ ID NO: 164 - protein, 487 aa, synthetic construct
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKVQPPGAFDIWGQGTMVTVSSGGGGSGGGGSGGGGSDIQLTQSPSFLSASVGDRVSITCRSTQGISSYLGWYQQKPGKAPKLLIYATSTLQSGVPSRFSGSGSGTEFTLTISSLQPEDFATYYCQQLYSFGQGTKLEIKGGGGSQVQLVQSGAEVKKPGASVKVSCKASGYTFTGYFMHWVRQAPGQGLEWMGWINSNSGGTNYAQKFQGRVTMTRDTSISTAYMELSRLRSDDTAVYYCARDDGTGYNAFDIWGQGTMVTVSSGGGGSGGGGSGGGGSDIVMTQSPLSLPVTPGEPASISCRSSQSLLHSNGYNYLDWYVQKPGKPPQLLIYLASHRASGVPDRFSGSGSGTDFTLKISRVEAEDVGIYYCMQAQQTALFGPGTKVDIK

SEQ ID NO: 165 - protein, 703 aa, synthetic construct
MLLLVTSLLLCELPHPAFLLIPEVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKVQPPGAFDIWGQGTMVTVSSGGGGSGGGGSGGGGSDIQLTQSPSFLSASVGDRVSITCRSTQGISSYLGWYQQKPGKAPKLLIYATSTLQSGVPSRFSGSGSGTEFTLTISSLQPEDFATYYCQQLYSFGQGTKLEIKGGGGSQVQLVQSGAEVKKPGASVKVSCKASGYTFTGYFMHWVRQAPGQGLEWMGWINSNSGGTNYAQKFQGRVTMTRDTSISTAYMELSRLRSDDTAVYYCARDDGTGYNAFDIWGQGTMVTVSSGGGGSGGGGSGGGGSDIVMTQSPLSLPVTPGEPASISCRSSQSLLHSNGYNYLDWYVQKPGKPPQLLIYLASHRASGVPDRFSGSGSGTDFTLKISRVEAEDVGIYYCMQAQQTALFGPGTKVDIKESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 166 - protein, 681 aa, synthetic construct
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKVQPPGAFDIWGQGTMVTVSSGGGGSGGGGSGGGGSDIQLTQSPSFLSASVGDRVSITCRSTQGISSYLGWYQQKPGKAPKLLIYATSTLQSGVPSRFSGSGSGTEFTLTISSLQPEDFATYYCQQLYSFGQGTKLEIKGGGGSQVQLVQSGAEVKKPGASVKVSCKASGYTFTGYFMHWVRQAPGQGLEWMGWINSNSGGTNYAQKFQGRVTMTRDTSISTAYMELSRLRSDDTAVYYCARDDGTGYNAFDIWGQGTMVTVSSGGGGSGGGGSGGGGSDIVMTQSPLSLPVTPGEPASISCRSSQSLLHSNGYNYLDWYVQKPGKPPQLLIYLASHRASGVPDRFSGSGSGTDFTLKISRVEAEDVGIYYCMQAQQTALFGPGTKVDIKESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 167 - protein, 415 aa, synthetic construct
MALPVTALLLPLALLLHAARPQVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDKWYGSAWRGDYWGQGTLVTVSSGGGGSGGGGSGGGGSQSVLTQPPSASGTPGQRVTISCSGSYSNIGSNFVNWYQQLPGTAPKLLIHFNNQRPSGVPDRFSGSRSGTSASLAISGLQSEDEADYFCSTWDDSLKGPIFGGGTKLTVLGGGGSGGGGSGGGGSAPASSSTKKTQLQLEHLLLDLQMILNGINNYKNPLLTDMLTRKFYMPKKATELKHLQCLEEELKPLEEVLNLAQSKNFHLRPRDLISNISNIVLELKGSETTFMCEYADETATIVEFLNRWITFSQSIISTLT

SEQ ID NO: 168 - protein, 394 aa, synthetic construct
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDKWYGSAWRGDYWGQGTLVTVSSGGGGSGGGGSGGGGSQSVLTQPPSASGTPGQRVTISCSGSYSNIGSNFVNWYQQLPGTAPKLLIHFNNQRPSGVPDRFSGSRSGTSASLAISGLQSEDEADYFCSTWDDSLKGPIFGGGTKLTVLGGGGSGGGGSGGGGSAPASSSTKKTQLQLEHLLLDLQMILNGINNYKNPLLTDMLTRKFYMPKKATELKHLQCLEEELKPLEEVLNLAQSKNFHLRPRDLISNISNIVLELKGSETTFMCEYADETATIVEFLNRWITFSQSIISTLT

SEQ ID NO: 169 - protein, 197 aa, synthetic construct
MALPVTALLLPLALLLHAARPAPASSSTKKTQLQLEHLLLDLQMILNGINNYKNPLLTDMLTRKFYMPKKATELKHLQCLEEELKPLEEVLNLAQSKNFHLSLREPIGNIVTSVRELKGSETTFMCEYADETATIVEFLNRWITFSQSIISTLTGSTSGSGKPGSGEGSTKGIYIWAPLAGTSGVLLLSLVITLYCN

SEQ ID NO: 170 - protein, 176 aa, synthetic construct
APASSSTKKTQLQLEHLLLDLQMILNGINNYKNPLLTDMLTRKFYMPKKATELKHLQCLEEELKPLEEVLNLAQSKNFHLSLREPIGNIVTSVRELKGSETTFMCEYADETATIVEFLNRWITFSQSIISTLTGSTSGSGKPGSGEGSTKGIYIWAPLAGTSGVLLLSLVITLYCN

SEQ ID NO: 171 - protein, 398 aa, synthetic construct
MALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGGSGGGSLQITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 172 - protein, 377 aa, synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGGSGGGSLQITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 173 - protein, 415 aa, synthetic construct
MALPVTALLLPLALLLHAARPQVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDKWYGSAWRGDYWGQGTLVTVSSGGGGSGGGGSGGGGSQSVLTQPPSASGTPGQRVTISCSGSYSNIGSNFVNWYQQLPGTAPKLLIHFNNQRPSGVPDRFSGSRSGTSASLAISGLQSEDEADYFCSTWDDSLKGPIFGGGTKLTVLGGGGSGGGGSGGGGSAPASSSTKKTQLQLEHLLLDLQMILNGINNYKNPLLTDMLTRKFYMPKKATELKHLQCLEEELKPLEEVLNLAQSKNFHLSLREPIGNIVTSVRELKGSETTFMCEYADETATIVEFLNRWITFSQSIISTLT

SEQ ID NO: 174 - protein, 394 aa, synthetic construct
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDKWYGSAWRGDYWGQGTLVTVSSGGGGSGGGGSGGGGSQSVLTQPPSASGTPGQRVTISCSGSYSNIGSNFVNWYQQLPGTAPKLLIHFNNQRPSGVPDRFSGSRSGTSASLAISGLQSEDEADYFCSTWDDSLKGPIFGGGTKLTVLGGGGSGGGGSGGGGSAPASSSTKKTQLQLEHLLLDLQMILNGINNYKNPLLTDMLTRKFYMPKKATELKHLQCLEEELKPLEEVLNLAQSKNFHLSLREPIGNIVTSVRELKGSETTFMCEYADETATIVEFLNRWITFSQSIISTLT

SEQ ID NO: 175 - protein, 135 aa, synthetic construct
MALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 176 - protein, 114 aa, synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 177 - protein, 154 aa, synthetic construct
MALPVTALLLPLALLLHAARPAPASSSTKKTQLQLEHLLLDLQMILNGINNYKNPLLTDMLTRKFYMPKKATELKHLQCLEEELKPLEEVLNLAQSKNFHLSLREPIGNIVTSVRELKGSETTFMCEYADETATIVEFLNRWITFSQSIISTLT

SEQ ID NO: 178 - protein, 133 aa, synthetic construct
APASSSTKKTQLQLEHLLLDLQMILNGINNYKNPLLTDMLTRKFYMPKKATELKHLQCLEEELKPLEEVLNLAQSKNFHLSLREPIGNIVTSVRELKGSETTFMCEYADETATIVEFLNRWITFSQSIISTLT

SEQ ID NO: 179 - protein, 154 aa, synthetic construct
MALPVTALLLPLALLLHAARPAPASSSTKKTQLQLEHLLLDLQMILNGINNYKNPLLTDMLTRKFYMPKKATELKHLQCLEEELKPLEEVLNLAQSKNFHLRPRDLISNISNIVLELKGSETTFMCEYADETATIVEFLNRWITFSQSIISTLT

SEQ ID NO: 180 - protein, 133 aa, synthetic construct
APASSSTKKTQLQLEHLLLDLQMILNGINNYKNPLLTDMLTRKFYMPKKATELKHLQCLEEELKPLEEVLNLAQSKNFHLRPRDLISNISNIVLELKGSETTFMCEYADETATIVEFLNRWITFSQSIISTLT

SEQ ID NO: 181 - protein, 9 aa, synthetic construct
  VARIANT 3: note = X is P or S
VQXPGAFDI